(19) United States
(12) Patent Application Publication
(10) Pub. No.: US 2025/0170274 A1
(43) Pub. Date: May 29, 2025

(54) GENE THERAPEUTICS FOR TREATING BONE DISORDERS

(71) Applicant: University of Massachusetts, Westborough, MA (US)

(72) Inventors: Jae-Hyuck Shim, Worcester, MA (US); Guangping Gao, Worcester, MA (US); Jun Xie, Worcester, MA (US); Jung Min Kim, Worcester, MA (US); Dan Wang, Worcester, MA (US); Yeon-Suk Yang, Worcester, MA (US)

(73) Assignee: University of Massachusetts, Westborough, MA (US)

(21) Appl. No.: 18/967,535

(22) Filed: Dec. 3, 2024

Related U.S. Application Data
(63) Continuation of application No. 16/982,640, filed on Sep. 21, 2020, now Pat. No. 12,194,108.
(60) Provisional application No. 62/799,843, filed on Feb. 1, 2019.
(60) Provisional application No. 62/647,595, filed on Mar. 23, 2018.

(51) Int. Cl. A61K 48/00; A61P 19/10; C07K 14/005; C07K 14/635; C12N 15/113; C12N 15/86

(52) U.S. Cl. CPC A61K 48/005; A61P 19/10; C07K 14/005; C07K 14/635; C12N 15/113; C12N 15/1137; C12N 15/86; C12N 2310/14; C12N 2310/141; C12N 2310/531; C12N 2320/32; C12N 2750/14122; C12N 2750/14143; C12N 2750/14145; C12N 2750/14171

(57) ABSTRACT

In some aspects, the disclosure relates to compositions and methods for modulating (e.g., increasing and/or decreasing) bone mass in a subject. In some aspects, the disclosure provides isolated nucleic acids, and vectors such as rAAV vectors, configured to express transgenes that promote (e.g., increase) or inhibit (e.g., decrease) activity, differentiation, or function of certain types of bone cells, for example osteoblasts, osteoclasts, osteocytes, etc. In some embodiments, the isolated nucleic acids and vectors described by the disclosure are useful for treating disorders and conditions associated with increased bone mass (e.g., osteopetrosis) or decreased bone mass (e.g., osteoporosis).

### RELATED APPLICATIONS

This Application claims the benefit under 35 U.S.C. § 119(e) of U.S. provisional Application No. 62/647,595, filed Mar. 23, 2018, entitled “GENE THERAPEUTICS FOR TREATING BONE DISORDERS” and U.S. provisional Application No. 62/799,843, filed Feb. 1, 2019, entitled “GENE THERAPEUTICS FOR TREATING BONE DISORDERS”.

### BACKGROUND

Defects in bone metabolism give rise to various different bone disorders, including disorders associated with a pathological depletion or bone mass and disorders associated with a pathological increase in bone mass. Effects on bone mass may be systemic or local. For example osteoporosis is a disease characterized by loss of bone mass, and is a major source of frailty and suffering associated with aging. An estimated 10 million Americans over age 50 have osteoporosis, and osteoporosis-related fractures occur in approximately 1.5 million individuals per year, with serious health consequences. Most existing therapeutic agents for osteoporosis inhibit resorption of bone by osteoclasts (OCs) and this inhibition is accompanied by numerous side effects, including atypical fractures and osteonecrosis of the jaw. Intermittent parathyroid hormone (PTH) is an anabolic agent that promotes osteoblast (OB) function and is available for the treatment of patients with osteoporosis. However, this agent is limited in its use because of the fear of PTH-induced bone tumors. Additionally, a newly developed anabolic agent, the anti-sclerostin antibody, promotes OB differentiation through enhanced Wnt signaling. However, in the context of inflammatory arthritis, anti-selerostin antibody has been observed to increase bone destruction in the context of TNF-dependent inflammation and elevate the risk of stroke. Similarly, a small molecule inhibitor of Cathepsin K (e.g., odanacatib) was withdrawn from FDA consideration due to an elevated incidence in stroke.

### SUMMARY

Aspects of the disclosure relate to compositions and methods for modulating (e.g., increasing or decreasing) bone formation and/or metabolism. The disclosure is based, in part, on recombinant adeno-associated viruses (rAAVs) that encode one or more transgenes that modulate bone metabolism. Thus, in some embodiments, rAAVs described by the disclosure are useful for treating a disease or disorder associated with dysregulated bone metabolism (e.g., decreased bone density, increased bone density, etc.).

Accordingly, in some aspects, the disclosure provides an isolated nucleic acid encoding: a first region comprising a first adeno-associated virus (AAV) inverted terminal repeat (ITR), or a variant thereof; and, a second region comprising a transgene encoding at least one bone metabolism modulating agent.

In some embodiments, a bone metabolism modulating agent is a bone formation promoting agent. In some embodiments, a bone formation promoting agent is selected from the group consisting of a protein that promotes OB and/or osteocyte (OCY) differentiation or activity, a protein that inhibits OC differentiation or activity, and an inhibitory nucleic acid that inhibits OC differentiation or activity.

In some embodiments, a bone metabolism-modulating agent is a bone formation inhibiting agent. In some embodiments, a bone formation-inhibiting agent is selected from the group consisting of a protein that inhibits OB and/or OCY differentiation or activity, a protein that promotes OC differentiation or activity, and an inhibitory nucleic acid that inhibits OB expression or activity.

In some embodiments, a transgene encodes a bone formation promoting agent selected from the group consisting of parathyroid hormone (PTH), PTH-related protein (PTHrP), deglycase DJ1, an inhibitory nucleic acid targeting selerostin (SOST), an inhibitory nucleic acid targeting schnurri-3 (SHIN3), and an inhibitory nucleic acid targeting cathepsin K (CTSK).

In some embodiments, a transgene encodes a bone formation inhibiting agent selected from the group consisting of sclerostin (SOST), schnurri-3 (SHIN3), cathepsin K (CTSK), an inhibitory nucleic acid targeting parathyroid hormone (PTH), an inhibitory nucleic acid targeting PTH-related protein (PTHrP), and an inhibitory nucleic acid targeting deglycase DJ1.

In some embodiments, a transgene encodes at least one inhibitory nucleic acid selected from the group consisting of dsRNA, siRNA, shRNA, miRNA, and artificial miRNA (amiRNA). In some embodiments, an inhibitory nucleic acid functions as a mutant terminal repeat (mTR).

In some embodiments, a transgene comprises a sequence set forth in any one of SEQ ID NOs: 1-15. In some embodiments, a transgene targets (e.g., hybridizes with or binds to) a sequence set forth in any one of SEQ ID NOs: 1-15, or a complement thereof.

In some embodiments, an isolated nucleic acid described by the disclosure further comprises at least one promoter that is operably linked to the transgene.

In some embodiments, an isolated nucleic acid described by the disclosure further comprises a third region comprising a second AAV ITR or a variant thereof.

In some aspects, the disclosure provides a vector comprising an isolated nucleic acid as described by the disclosure. In some embodiments, a vector is a plasmid.

In some aspects, the disclosure provides a host cell comprising an isolated nucleic acid or vector as described by the disclosure. In some embodiments, a host cell is a bacterial cell, yeast cell, insect (e.g., Sf9) cell, or a mammalian cell.

The disclosure is based, in part, on rAA Vs that are characterized by an increased tropism for bone cells, such as OBs, OCYs, OCs, etc. In some embodiments, rAAVs described by the disclosure comprise a heterologous bone-targeting peptide or are conjugated to a bone-targeting moiety.

Accordingly, in some aspects, the disclosure provides an isolated nucleic acid encoding a recombinant adeno-associated virus (rAAV) capsid protein comprising a heterologous bone-targeting peptide. In some embodiments, a heterologous bone-targeting peptide targets OCs (e.g., specifically, or preferentially targets OCs relative to OBs). In some embodiments, a heterologous bone-targeting peptide targets OBs (e.g., specifically, or preferentially targets OBs relative to OCs). In some embodiments, a heterologous bone-targeting peptide comprises the amino acid sequence set forth in SEQ ID NOs: 16, 17, 57, 58, 59, 60, 61, 62, and 63.

In some aspects, the disclosure provides an rAAV capsid protein comprising one or more azide-bearing unnatural amino acids. In some embodiments, a capsid protein is conjugated to one or more alendronate (Ale) moiety via one or more azide-bearing unnatural amino acids.

In some aspects, the disclosure provides a recombinant adeno-associated virus (rAAV) comprising: a capsid protein; and, an isolated nucleic acid as described by the disclosure.

In some embodiments, a capsid protein is of a serotype selected from AAVI, AAV2, AAV3, AAV4, AAV5, AAV6, AAV6.2, AAV7, AAV8, AAV9, AAV.rh8, AAV.xl10, AAV.rh39, AAV.43, AAV2/2-66, AAV2/2-84, and AAV2/2-125, or a variant of any of the foregoing SEQ ID NOs: 18-34.

In some embodiments, a capsid protein transduces OBs and/or OCYs. In some embodiments, a capsid protein (e.g., a capsid protein that transduces OBs and/or OCYs) is of a serotype selected from AAV4, AAV1, AAV6, AAV6.2, and AAV9, or a variant of any of the foregoing.

In some embodiments, a capsid protein transduces OCs. In some embodiments, a capsid protein (e.g., a capsid protein that transduces OCs) is of a serotype selected from AAV1, AAV5, AAV6, AAV6.2, AAV7, AAV8, AAV9, AAV10, AAV.xh39, and AAV.rh43, or a variant of any of the foregoing.

In some embodiments, a capsid protein comprises a heterologous bone-targeting peptides, for example heterologous bone-targeting peptide comprising the amino acid sequence set forth in SEQ ID NO: 16, 17, 57, 58, 59, 60, 61, 62, and 63. In some embodiments, a heterologous bone-targeting peptide targets OCs (e.g., specifically, or preferentially targets OCs relative to OBs). In some embodiments, a heterologous bone-targeting peptide targets OBs (e.g., specifically, or preferentially targets OBs relative to OCs).

In some embodiments, a nucleic acid sequence encoding heterologous bone-targeting peptides is inserted into a VP2 open reading frame of a capsid protein. In some embodiments, the nucleic acid sequence is inserted between codons corresponding to N587 and R588 or N-terminus of a nucleic acid sequence encoding an AAV2 capsid protein. In some embodiments. the nucleic acid sequence is inserted between codons corresponding to Q588 and AS89 or N-terminus of a nucleic acid sequence encoding an AAV9 capsid protein.

In some embodiments, a capsid protein is encoded by an amino acid sequence having one or more azide-bearing unnatural amino acids. In some embodiments, a capsid protein is conjugated to one or more alendronate (Ale) moiety via one or more azide-bearing unnatural amino acids.

In some embodiments, an rAAV is a self-complementary AAV (scAAV).

In some aspects, the disclosure provides a method for delivering a transgene to bone tissue, the method comprising administering to a subject an isolated nucleic acid, composition, or rAAV as described by the disclosure.

In some aspects, the disclosure provides a method for treating a disease or disorder associated with reduced bone density, the method comprising administering to a subject having or suspected of having a disease or disorder associated with reduced bone density as described herein, wherein the transgene of the rAAV encodes a bone formation promoting agent. In some embodiments, the bone formation promoting agent is selected from the group consisting of a protein that promotes OB and/or OCY differentiation or activity, a protein that inhibits OC differentiation or activity, and an inhibitory nucleic acid that inhibits OC differentiation or activity.

In some embodiments, the bone formation promoting agent selected from the group consisting of parathyroid hormone (PTH), PTH-related protein (PTHrP), deglycase DJ1, an inhibitory nucleic acid targeting sclerostin (SOST), an inhibitory nucleic acid targeting Schnurri-3 (SHN3), and an inhibitory nucleic acid targeting cathepsin K (CTSK).

In some embodiments, a disease or disorder associated with reduced bone density is selected from the group consisting of osteoporosis, a critical sized-bone defect, a mechanical disorder resulting from disuse or injury, and secondary disorders such as breast cancer or prostate cancer metastasis, type 1 diabetes, lupus, rheumatoid arthritis, inflammatory bowel disease, hyperthyroidism, celiac disease, asthma, periodontitis, and multiple sclerosis.

In some aspects, the disclosure provides a method for treating a disease or disorder associated with increased bone density, for example a disease selected from the group consisting of osteopetrosis, osteosarcoma, and heterotropic ossification, the method comprising administering to a subject having or suspected of having a disease or disorder associated with increased bone density an rAAV as described herein, wherein the transgene of the rAAV encodes a bone formation inhibiting agent. In some embodiments, the bone formation inhibiting agent is selected from the group consisting of a protein that inhibits OB and/or OCY differentiation or activity, a protein that promotes OC differentiation or activity, and an inhibitory nucleic acid that inhibits OC differentiation or activity.

In some embodiments, the bone formation inhibiting agent selected from the group consisting of sclerostin (SOST), schnurri-3 (SHN3), cathepsin K (CTSK), an inhibitory nucleic acid targeting parathyroid hormone (PTH), an inhibitory nucleic acid targeting PTH-related protein (PTHrP), and an inhibitory nucleic acid targeting deglycase DJ1.

In some embodiments, a disease or disorder associated with reduced bone density is selected from the group consisting of osteopetrosis, pyenodysostosis, selerosteosis, acromegaly. fluorosis, myelofibrosis, hepatitis C-associated osteosclerosis, and cancers of bone such as osteosarcoma and metastatic cancer of the bone.

In some embodiments of methods described by the disclosure, administration occurs by injection. In some embodiments, injection is systemic injection (e.g., tail vein injection), local injection (e.g.,, intramuscular (IM) injection, knee injection, or femoral intramedullary injection).

In some embodiments, administration occurs by implantation of a tissue or graft comprising an rAAV as described by the disclosure into a subject.

In some embodiments, administration results in transduction of a cell type selected from the group consisting of OB, OCY, OC, and chondrocyte,

### BRIEF DESCRIPTION OF DRAWINGS

FIG. 1 shows identification of scAAV serotypes that transduce mouse primary OB precursors in vitro. Mouse OB precursors were isolated from the calvaria of neonates at postnatal day 3-5 and cultured in the growth media for amplification. Cells were incubated with 14 different scAAV serotypes encoding GFP proteins for 2 days and their transduction efficiency was analyzed by GFP expression using epifluorescence microscopy.

FIG. 2 shows identification of scAAV serotypes that transduce mouse ATDC5 chondrocyte line in vitro. Mouse chondrocyte progenitor cell line ATDCS was incubated with 14 different scAAV serotypes encoding GFP proteins for 2 days and their transduction efficiency was analyzed by GFP expression using epifluorescence microscopy.

FIG. 3 shows identification of scAAV serotypes that can transduce mouse primary OCs in vitro. Bone marrow-derived monocytes (BM-MOs) were isolated from the long bones of 2-month old mice (C57BL/6J) and amplified by the addition of mouse m. M-CSF (40 ng/ml). BM-MOs were incubated with 15 different scAAV serotypes encoding GFP proteins in the presence of mouse M-CSF (40 ng/ml) and mouse Rank ligand (10 ng/ml) for 6 days their transduction efficiency was analyzed by GFP expression using epifluorescence microscopy.

FIG. 4 shows identification of scAAV serotypes that can transduce mouse Raw264.7 OC line in vitro. 2 days after the treatment with mouse Rank ligand (5 ng/ml), Raw264.7 cells were incubated with 17 different scAAV serotypes encoding GFP proteins in the presence of mouse Rank ligand (5 ng/ml) for 6 days. Their transduction efficiency was analyzed by GFP expression using epifluorescence microscopy.

FIG. 5 shows optical images of GFP-expressing AAV-transduced tissues in mice. A single dose of PBS or 1012 ˜1013/ml genome copies of GFP-encoding scAAV serotypes (scAAV4, 5, 6.2, and 9) was injected into both knee joints of 2 month old mice (C57BL/6J) and 4 weeks later, mice were euthanized, and GFP expression in the knee joints was monitored by the IVIS-100 optical imaging system (whole body, top). The right hindlimbs were dissected and after removal of muscle GFP expression was monitored by the IVIS-100 optical imaging system (hindlimb, bottom).

FIGS. 6A-6B show identification of scAAV serotypes that can transduce chondrocytes in articular cartilage and/or OBs and/or OCs on the bone surface. A single dose of 10¹²˜10¹³/ml genome copies of GPP-encoding scAAV serotypes (scAAV4, 5, 6.2, and 9) was injected into both knee joints of 2 month old mice (C57BL/61) and 4 weeks later, knee joints (FIG. 6A) and femurs (FIG. 6B) were frozen-sectioned for histology. AC; articular cartilage, CB: cortical bone, BM: bone marrow, Bar: 50 pm.

FIG. 7 shows that liposomes can enhance the selectivity of scAAV9-transduction to bone cells by reducing its infectivity to muscle in mice. 10¹²˜10¹³/ml genome copies of GFP-encoding scAAV9 serotype were mixed with PBS or X-tremeGENE (liposome, Roche) at 1:1 ratio. I hour after incubation, a single dose of the mixture was injected into knee joints of 2month old mice (C57BL/6J). 1 week later, mice were euthanized, and GFP expression in the knee joints was monitored by the IVIS-100 optical imaging system (left). Femurs were frozen-sectioned for histology (right). CB: cortical bone, M: muscle.

FIG. 8 shows GFP-encoding scAAV9 serotype can transduce OBs, and OCs, and OCYs on the bone surface in mice. 10¹²˜10¹³/ml genome copies of GFP-encoding scAAV9 serotype were mixed with PBS or X-tremeGENE (liposome) at 1:1 ratio. 1 hour after incubation, a single dose of the mixture was injected into knee joints of 2 month old mice (C57BL/6J). 1 A single dose of 10¹²˜10³³/ml genome copies of GFP-encoding scAAV9 serotype was injected into knee joints of 2 month old mice (C57BL/6J) and 4 weeks later, femurs were frozen-sectioned for histology and GFP expression was viewed by a confocal microscope. High-powered pictures show scAAV9-transduced OBs. OCs, and OCYs in the femar treated with a mixture of GFP-encoding scAAV9 serotype with X-tremeGENE (bottom). GFP expression was observed in the cortical bone viewed by a phase-contrast microscope (left). GP: growth plate, TB: trabecular bone, CB: cortical bone, BM: bone marrow; Bar: 50 μm.

FIG. 9 shows development of bone-seeking AAV vectors. (A) Bone-targeting peptides, DSS ((AspSerSer)₆ or HABP,) or (CγEPRRγEVAYELγEPRRγEVAγEL) are inserted into the VP2 capsid protein via a genetic manipulation. (B) WT AAV vector are site-specifically labeled with azide-bearing amino acids and then attached with a bone-seeking molecule (ADIBO-Ale).

FIG. 10 shows a diagram depicting the VP2 capsid protein with bone-targeting peptides, (box; (AspSerSer) 6, DSS). DSS are inserted into between Q588 and A589 or the N-terminus of the VP2 capsid protein via a genetic manipulation.

FIGS. 11A-11C show in vitro characterization of DSS-seAAV9s in OBs. Mouse OB precursors were incubated with scAAV9-WT (WT) or two DSS-scAAV9s (DSS-588, DSS-Nter) for 2 days. Their transduction efficiency was analyzed by GFP expression using western blotting with anti-GPP (FIG. 11A) and epifluorescence (FIG. 11B, left). Hsp90 was used as a loading control. Alternatively, transduced OBs were cultured under OB differentiation conditions for 6 days and OB differentiation was assessed by alkaline phosphatase (ALK) staining (FIG. 11B, right) and OB gene expression by RT-PCR (normalized to Hprt, FIG. 11C). N.S.; not significant.

FIGS. 12A-12C show in vitro characterization of DSS-scAAV9s in OCs. 2 days after the treatment with mouse Rank ligand (5 ng/ml), Raw264.7 were treated with scAAV9-WT (WT) or two DSS-scAAV9s (DSS-588, DSS-Nter). 2 days after transduction, their transduction efficiency was analyzed by GFP expression using western blotting with anti-GFP antibody (FIG. 12A) using epifluorescence (FIG. 12B, left). Transduced OCs were cultured in the presence of Rank ligand and 3 days later, OC differentiation was assessed by TRAP staining (FIG. 12B, right) and OC gene expression by RT-PCR (normalized to Hprt, FIG. 12C). N.S: not significant.

FIG. 13 shows that DSS-scAAV9 (Nter) can transduce OBs. OCs, and OCYs on the bone surface in mice. 10¹¹˜10¹²/ml genome copies of GFP-encoding seAAV9-WT (WT) or two DSS-scAAV9s (DSS-588, DSS-Nter) were mixed with X-tremeGENE (liposome) af 1:1 ratio, 1 hour after incubation, a single dose of the mixture was injected into knee joints of 2 month old mice (C57BL/6J). 1 week later, femurs were frozen-sectioned for histology and GFP expression was viewed by a confocal microscope. High-powered pictures show seAAV9-transduced OBs. OCs, and OCYs in the femur treated with a mixture of GFP-encoding scAAV9serotype with X-tremeGENE (bottom). GP: growth plate, CB: cortical bone, BM: bone marrow; GFP: green, DAPI: blue,

FIG. 14 shows that transient deletion of SHN3 in mature OBs increases bone mass in adult mice. 1 month-old female Shn3-fl/fl (WT) or Shn3-fl/fl;OCN/ERT-cre (CKO) mice were treated with tamoxifen (50 mg/kg) for 5 days and 6 weeks later bone mass was measured by microCT analysis. Displayed are 3D reconstructions of trabecular bones (left) and quantitative parameters are displayed in the right panel: bone volume/total volume (BV/TV). **: P.0.005.

FIG. 15l shows that SHN3 deletion prevents estrogen deficiency-induced bone loss in mice. 3 month-old female mice have undergone ovariectomy (OVX) surgery or have undergone Sham surgery (Sham) and 2 month after the surgery bone mass was measured by microCT analysis. Displayed are 3D reconstructions of trabecular bones (left top) and midshaft cortical bones (left bottom). Quantitative parameters are displayed in the right panel: bone volume/total volume (BV/TV). **: P,0.005, N.S: non-significant.

FIGS. 16A-16D show in vitro characterization of scAAV9-mSHN3i in OBs. Mouse primary calvarial OBs were treated with scAAV9 encoding-control vector (control) or two mouse-SHN3 shRNAsi (Sh-SHN3-1, -2). 3 days after transduction, their transduction efficiency (FIG. 13A) and knockdown efficiency (FIG. 13B) were analyzed by GFP expression using epifluorescence and by SHN3 mRNA levels using RT-PCR, respectively. (FIGS. 13C and 13D) Transduced OBs were cultured under OB differentiation conditions RT-PCR and alizarin red staining were performed at 6 and 15 days of the culture. *: P,0.05, #*: P.0.005.

FIGS. 17A-17B show in vivo characterization of scAAV9-mSHN3i. 10¹²˜10¹³/ml genome copies of GFP-encoding scAAV9 (control, Sh-SHN3-1) were mixed with X-tremeGENE (liposome) at 1:1 ratio. 1 hour after incubation, a single dose of the mixture was injected into knee joints of 2 month old mice (C57BL/6J). 1 week later, femurs were frozen-sectioned for histology and GFP expression was viewed by a confocal microscope. High-powered pictures show GFP-expressing OBs and OCYs in the femur, demonstrating that similar to control-scAAV9 vector, Sh-SHN3-1 scAAV9 vector can transduce OBs and OCYs in the bone (FIG. 17A). Femurs were processed with mechanical and enzymatic digestion (type 2collagenase and dispase) to dissociate BMSCs. GFP-expressing cells were further isolated from BMSCs using FACS sorting and used for RT-PCR analysis to measure SHN3 mRNA levels (FIG. 17B). CB: cortical bone, BM: bone marrow, TB: trabecular bone.

FIGS. 18A-18D show in vitro characterization of scAAV9-mCTSKi in OCs. 2 days after the treatment with mouse Rank ligand (5 ng/ml), Raw264.7 were treated with scAAV9-control (control) or two scAAV9-mCTSKi (Sh-CTSK-1, -2). 2 days after transduction, their transduction efficiency (FIG. 18A) and knockdown efficiency (FIG. 18B) were analyzed by GFP expression using epifluorescence and by CTSK mRNA levels using RT-PCR, respectively. FIGS. 18C-18D show transduced OBs were cultured in the presence of Rank ligand and 3 days later, OC differentiation was assessed by TRAP activity (FIG. 18C, top) and staining (FIG. 18C, bottom) and OC gene expression by RT-PCR (normalized to Hprt, FIG. 18D). *: P,0.05, **; P,0.005.

FIGS. 19A-19B show in vivo characterization of scAAV9-mCTSKi. 10¹²˜10¹³/ml genome copies of GFP-encoding scAAV9 (control, Sh-CTSK-1) were mixed with X-tremeGENE (liposome) at 1:1 ratio. 1 hour after incubation, a single dose of the mixture was injected into knee joints of 2 month old mice (C57BL./61). 1 week later, femurs were frozen-sectioned for histology and GFP expression was viewed by a confocal microscope. High-powered pictures show scAAV9-transduced OBs and OCs in the femur (FIG. 19A). Frozen-sectioned femurs were stained with anti-CTSK antibody to assess CTSK expression in GFP-expressing OCs. IgG antibody was used as a negative control. High-powered pictures show a reduced expression of CTSK in GFP-expressing OCs transduced with scAAV9-mCTSKi (FIG. 19B). OP: growth plate, CB: cortical bone, BM: bone marrow, TB: trabecular bone; GFP; green, DAPI: blue, CTSK: red.

FIG. 20 shows a strategy of bone-targeting scAAV9-mediated gene silencing. ShRNAs specific to OB genes (SHN3i, SOSTi) or to OC gene (CTSKi) are cloned into BT-scAAV9 serotypes (DSS or HABP-scAAV9, Ale-scAAV9). These scAAV9 serotypes are injected into the intramedullary femur of mice for local delivery. For systemic delivery, they are IP or IV injected into mice in order to silence expression of SHN3 or SOST in OBs or osteocytes or to silence CTSK expression in OCs on the bone surface.

FIG. 21 shows AAV-mediated gene addition for systemie production. Human PTH (1-84 aa), human PTHrP (1-140 aa), or mouse DJ-1 cDNA was cloned into scAAV9 vectors and they are intramuscularly injected to mice for systemic expression.

FIGS. 22A-22G shows scAAV vectors that transduce bone cells in vitro and in vivo. FIGS. 22A and 22B show calvarial osteoblasts (COB), bone marrow-derived osteoclast precursors (BM-OCP). or chondrogenic cells (ATDC5), were treated with PBS or 14 different AAV capsids packaged with the same CB-Egfp transgene. Two days later, EGFP expression was assessed by immunoblotting with an anti-GFP antibody. The anti-Hsp90 antibody was used as a loading control. Immunoblot quantification of EGFP protein was measured as a percentage of endogenous Hsp90 protein level by Image J software. FIG. 22C shows a single dose of 1× 100 genome copies of scAAV was intraarticularly (i.a.) injected into the knee joints of two-month-old male mice, and EGFP expression in the hindlimb was monitored by IVIS-100 optical imaging. FIG. 22D shows femurs were cryo-sectioned and EGFP expression was assessed by fluorescence microscopy. FIGS. 22E and 22F show high-magnification images of EGFP-expression osteoblasts (OB) osteocytes (OCY), and mature osteoclasts (OC). FIG. 22G Cryo-sectioned femurs were also immunostained with an anti-Bglap antibody to identify mature osteoclast. TB, trabecular bone; BM, bone marrow; GP, growth plate; CB, cortical bone. Scale bars: 500 μm (FIG. 22D); 100 μm (FIG. 22E); 75 μm (left) and 25 μm (middle, right) (FIG. 22F); and 25 μm (FIG. 22G).

FIGS. 23A-23K shows inducible deletion of Shn3 in osteoblasts increases bone accrual in adult mice. In FIGS. 23A-23D, a single dose of 4×10¹¹ genome copies of scAAV9-Egfp was intravenously (i.v.) injected into two-month-old male mice. FIG. 23A shows EGFP expression in individual tissues monitored by IVIS-100 optical imaging two weeks post-injection. y-axis, radiant efficiency (p/sec/cm²/sr/μW/cm²). FIGS. 23B and 23C shows EGFP expression in cryo-sectioned heart and liver (23B) and femur (23C, high magnification on the right). FIGS. 23D is an immunoblot of tissue lysates with an anti-EGFP antibody. In FIGS. 23E-23G, two-month-old female Ocn-Ert;Rosa^{mT/mG} and Shn3^{Oen-En};Rosa^{mT/mG} mice were treated with 100 mg/kg tamoxifen for 5 consecutive days. In FIG. 23E, femurs were cross-sectioned two months later to identify EGFP-expressing osteoblasts, FIGS. 23F and 23G shows a representative 3D construction of femoral trabecular bone mass assessed by microCT (FIG. 23F) and relative quantification (FIG. 23G). Trabecular bone volume/total volume (Tb.BV/TV), trabecular thickness (Tb.Th), trabecular number per cubic millimeter (Tb.N) and trabecular space (Tb.Sp) (n=6/group). In FIGS. 23H-23K, a single dose of 4×10¹¹ genome copies of scAAV9-Egfp or scAAV9-Cre was i.v. injected into three-month-old male Shn3^{fl/fl};Rosa^{mTmG} mice. FIG. 23H shows Cre and Shn3 mRNA levels in tibial bone RNA and normalized to Aprt expression two months after treatment. FIG. 23I shows fluorescence microscopy on cryo-sectioned femurs to identify EGFP-expressing cells (FIG. 23I), and femoral trabecular bone mass was assessed by microCT. FIG. 23J shows representative 3D-reconstruction and FIG. 23K shows relative quantification. Trabecular bone volume/total volume (Tb. BV/TV), trabecular thickness (Tb.Th), trabecular number per cubic millimeter (Tb.N), and consecutive density (Conn.Dn) (n=6/group). Scale bars: 50 μm (23B); 500 μm (left), 75 μm (right) (23C): 25 μm (23E); 1 mm (23F and 23J); 250 μm (23I). Values represent mean±standard deviation: *, P<0.05; **, P<0.01; and ***, P<0.001 by an unpaired two-tailed Student's t-test (23G, 23H, and 23K).

FIGS. 24A-24K show the silencing of SHN3 by systematically delivered AAV9 promotes bone formation. FIG. 24A shows a diagram of the scAAV9 construct containing a CMV enhancer/chicken β-actin promoter (CB), amiR-ctrl or amiR-shn3, an Egfp reporter gene (EGFP), β-globin polyA sequence (PA), and inverted terminal repeat (ITR). In FIGS. 24B-24D, two weeks after intraarticular (i.a.) injection of scAAV9 encoding amiR-ctrl or amiR-shn3 into knee joints of two month-old male mice, EGFP expression was assessed by IVIS 100 optical imaging (FIG. 24B) and fluorescence microscopy of cryo-sectioned femurs (FIG. 24C). In FIG. 24D, levels of EGFP protein (top) and shn3 mRNAs are normalized to hprt (bottom) were assessed in fluoresence activated cell sorted (FACS)-sorted EGFP-expressing cells from the femur. In FIGS. 24E-24F, two months after i.a. injection of scAAV9 carrying amiR-ctrl or amiR-shn3 into knee joints of two-month-old female mice, femoral trabecular bone mass was assessed by microCT. Representative 3D-reconstruction (FIG. 24E) and relative quantification (FIG. 24F) are displayed (n=6/group). In FIGS. 24G-24K, a single dose of 4×10¹¹ genome copies of AAV9 encoding amiR-ctrl or amiR-shn3 were i.v. injected into three-month-old female mice. Two months later, shn3 mRNA levels were measured in the tibial bone RNA and normalized to hipri (FIG. 24G, n=8/group). Femoral trabecular bone mass was measured by microCT. Representative 3D-reconstruction (FIG. 24H) and relative quantification (FIG. 24I) are displayed (n=8/group). Representative calcein/alizarin red labeling (FIG. 24K) and relative histomorphometric quantification of BER/BS, MAR, and Ob.S/BS are displayed (FIG. 24K). Arrows indicate the distance between calcein and alizarin red labeling. BFR/BS, bone formation rate/bone surface; MAR, mineral apposition rate; Ob.S/BS, osteoblast surface/bone surface. GP, growth plate; BM, bone marrow; TB, trabecular bone. Scale bars: 250 μm. (FIG. 24C); 1 mm (FIGS. 24E and 24H); 50 μm (FIG. 24J). Values represent mean #SD: **, P<0.01; ***, P<0.001; and ****, P<0.0001 by an unpaired two-tailed Student's t-test (FIGS. 24F, 24G, 24I, and 24K).

FIGS. 25A-25J show silencing of Shn3 by systemically delivered AAV9 prevents bone loss in a mouse model of postmenopausal osteoporosis. FIGS. 25A-25B show sham or OVX surgery was performed on three-month-old female Shn3^{+/+} and Shn3^{-/-} mice and two months later, femoral trabecular bone mass was assessed by microCT. Representative images of the femur (FIG. 25A) and relative BV/TV (FIG. 25B) are displayed (n=6/group). FIG. 25C shows a diagram of the study and treatment methods. Sham or OVX surgery was performed on three-month-old female mice and six weeks later, a single dose of 4×10¹¹ genome copies of seAAV9 carrying amiR-ctrl or amiR-shn3 was i.v. injected. Seven weeks after injection, femurs were cryo-sectioned to identify EGFP-expressing cells (FIG. 25D). shn3 mRNA levels in tibial bone are displayed after normalization to hprt (n=8˜12/group) (FIG. 25E). Femoral trabecular bone mass was assessed by microCT. Representative 3D-reconstruction (FIG. 25F) and relative quantification (FIG. 25G) are displayed (n=7˜8/group). Representative images of calcein/alizarin red labeling (FIG. 25H) and relative histomorphometric quantification of BFR/BS and MAR (FIG. 25I). Arrows indicate the distance between calcein and alizarin red labeling. Femoral biomechanical properties, including bending rigidity, bending moment, apparent bending modulus, and apparent bending stress were quantified (n=5-9/group) (FIG. 25J). Scale bars: 1 mm, (25A and 25F); 250 μm, (FIG. 25D); and 50 μm, (FIG. 25H). Values represent mean±SD: N.S., not significant; *, P<0.05; **, P<0.01; ***, P<0.001; and **** P<0.0001 by an unpaired two-tailed Student's t-test (FIG. 25B) and one-way ANOVA test (FIGS. 25E. 25G, 25I, and 25J).

FIGS. 26A-26M: The bone-homing AAV9. DSS-Nter capsid mediates silencing of Shn3 to prevent bone loss in a mouse model of postmenopausal osteoporosis. FIG. 26A is a diagram of constructs for rationally designed bone-specific AAV capsids. The bone-targeting peptide motif (DSS, red) was inserted into the AAV9 capsid between Q588 and A589 (DSS-588) or at the N-terminus of AAV9-VP2 (DSS-Nter). cap: capsid proteins. FIGS. 26B and 26C shows that two days after infection with different concentrations of scAAV9, scAAV9. DSS-587, or scAAV9. DSS-Nter, COBs were cultured under osteogenic conditions for six days. EGFP expression was assessed by immunoblotting with an anti-EGFP antibody (FIG. 26B) or fluorescence microscopy (FIG. 26C). ALP activity was assessed by fast blue staining (FIG. 26C). FIGS. 26D-26H show a single dose of 4×10¹¹ genome copies of scAAV9 or scAAV9. DSS-Nter was i.v. injected into two-month-old male mice. EGFP expression in individual tissues was monitored by IVIS-100 optical imaging two weeks post-injection (FIG. 26D), Immunoblotting shows EGFP expression in tissue lysates (FIG. 26E) and relative quantification by the Image J software (FIG. 26F, n=3/group). Fluorescence microscopy was performed on cryo-sectioned femurs to identify EGFP-expressing cells (FIG. 26G) and the number of EGFP-expressing cells per bone surface in femurs were quantified by the Image J software (FIG. 26H, n=5/group). (FIGS. 26I-26M) show sham or OVX surgery was performed on three-month-old female mice. Six weeks later, a single dose of 4×10¹¹ genome copies of scAAV9. DSS-Nter carrying amiR-ctrl or amiR-shn3 was i.v. injected. Seven weeks after injection, slut3 mRNA levels were assessed in the tibial bone (FIG. 26I, n=10/group). Trabecular bone mass in the femur and lumbar vertebrae was assessed by microCT. Quantification (FIGS. 26J and 26M) and representative 3D-reconstruction (FIGS. 26K and 26L) are displayed (n=6˜8/group). GP. growth plate; BM, bone marrow; TB, trabecular bone. Scale bars: 100 μm. (FIG. 26C and 26G); 1 mm, (FIGS. 26K and 26L). Values represent mean±SD: N.S., not significant; *, P<0.05; **, P<0.01; ***, P<0.001; and ****, P<0.0001 by an unpaired two-tailed Student's t-test (FIGS. 26F and 26H) and one-way ANOVA test (FIGS. 26I, 26J, and 26M).

FIGS. 27A-27C show the identification of scAAV serotypes that transduce osteoblasts, osteoclasts, and chondrocytes in vitro. Calvarial osteoblasts (COBs, FIG. 27A), bone marrow-derived osteoclast precursors (BM-OCP, FIG. 27B), or a chondrogenic cell line (ATDC5, FIG. 27C) were treated with PBS or 14 different AAV serotypes. Two days later, EGFP expression was monitored by fluorescence microscopy. Scale bar: 100 μm.

FIGS. 28A-28B show the identification of scAAV serotypes that transduce bone and cartilage. A single dose of PBS or 1×10¹¹ genome copies of scAAV9-EGFP was intraarticularly (i.a.) injected into knee joints of two-month-old male mice. Knee joints (FIG. 28A) and femurs (FIG. 28B) were cryo-sectioned to identify EGFP-expressing cells. DAPI was used to stain nuclei. AC, articular cartilage; M, muscle; CB, cortical bone; BM, bone marrow. Scale bars: 100 μm, panel (FIG. 28A; 250 μm, FIG. 28B),

FIGS. 29A-29D show the tissue distribution of systematically-delivered scAAV9 in mice. FIGS. 29A and 29B show a single dose of PBS or 4×10¹¹ genome copies of scAAV9. EGFP was intravenously (i.v.) injected into two-month-old male mice and EGFP expression was monitored by IVIS-100 optical imaging two weeks post-injection. EGFP expression in whole body (FIG. 29A) and the quantification of EGFP expression in the dissected tissues shown in FIG. 28A are displayed (FIG. 29B). PBS-injection was used as a negative control. FIGS. 29C and 29D show tissues dissected from PBS-or rAAV9-EGFP-injected mice were cryo-sectioned to locate EGFP-expressing cells. Scale bars: 100 μm, FIG. 29C; 500 μm, FIG. 29D.

FIG. 30 shows the distribution of systematically-delivered seAAV9 in the femur. A single dose of 4×10¹¹ genome copies of scAAV-EGFP was i.v. injected into two-month-old male mice and EGFP expression was assessed in the cryo-sectioned femurs two weeks post-injection. L, ligament; GP, growth plate; AC, articular cartilage; BM, bone marrow; CB, cortical bone; P, patella; M, muscle. Scale bars: 100 μm.

FIGS. 31A-31E show the in vitro characterization of scAAV9-Cre in Shn3^{fl/fl} osteoblasts. FIG. 31A shows a diagram of rAAV9 constructs containing the CMV enhancer/chicken β-actin promoter (CB), an EGFP reporter gene (EGFP), or Cre recombinase (Cre), β-globin polyA sequence (PA), and inverted terminal repeat (ITR). FIG. 31B shows that COBs infected with scAAV9-EGFP or scAAV9-Cre for two days and cells were lysed and immunoblotted with the indicated antibodies. FIGS. 31C-31E show two days after treatment with scAAV9-EGFP or scAAV9-Cre, COBs were cultured under osteogenic conditions for six days and mRNA levels of Shn3 (FIG. 31C) and osteogenic genes (FIG. 31D) were measured by RT-PCR. After 21 days of the culture, mineralization was assessed by alizarin red staining (FIG. 31E). Values represent mean±SD: ***, P<0.001 by an unpaired two-tailed Student's t-test (FIGS. 31C and 31D).

FIGS. 32A-32C show the in vitro characterization of scAAV9 carrying amiR-shn3 in osteoblasts. Two days after treatment with scAAV9 carrying amiR-ctrl or amiR-shn3, COBs were cultured under osteogenic conditions. EGFP expression (FIG. 32A), mRNA levels of Shn3and osteogenic gene (FIG. 32B), and mineralization (FIG. 32C), were assessed by fluorescence microscopy, RT-PCR, and alizarin red staining, respectively. Scale bar: 100 mm, (FIG. 32A). Values represent mean±SD: *, P<0.05: ***, P<0.001; and ****, P<0.0001 by an unpaired two-tailed Student's t-test (FIGS. 32B and 32C).

FIGS. 33A-33F show the characterization of mice treated with systematically delivered scAAV9 carrying amiR-shn3. A single dose of 4×10¹¹ genome copies of scAAV9 carrying amiR-ctrl or amiR-shn3 was i.v. injected into three-month-old female mice. Two months following treatment, mice were labeled with calcein and alizarin red for dynamic histomorphometry. Non-labeled mice were used to monitor EGFP expression using IVIS-100optical imaging. EGFP expression in the whole body (FIG. 33A) and the dissected tissues (FIG. 33B) are displayed. Femurs were cryo-sectioned to identify EGFP-expressing osteoblast lineage cells (FIG. 33C). TRAP-stained longitudinal sections (FIG. 33D) and histomorphometric analysis (FIG. 33E) of femurs from five-month-old female mice treated with scAAV9 carrying amiR-ctrl or amiR-shn3 (n=6˜7). The number of osteoclasts per bone perimeter (N.Oc/B.Pm). Serum CTX levels were assessed by ELISA (n=6) (FIG. 33F). Scale bars: 100 μm, FIG. 33C; 50 mm, FIG. 33D. Values represent mean±SD; N.S., not significant by an unpaired two-tailed Student's t-test (FIGS. 33E and 33F).

FIGS. 34A-34C show the therapeutic effects of systematically delivered scAAV9-amiR-shn3 in a mouse model of postmenopausal osteoporosis. Sham or OVX surgery was performed on three-month-old female mice and six weeks later, a single dose of 4×10¹¹ genome copies of scAAV9 carrying amiR-ctrl or amiR-shn3 was i.v. injected. Seven weeks after injection, mice were labeled with calcein and alizarin red for dynamic histomorphometry. Staining by Von Kossa (FIG. 34A) or TRAP (FIG. 34B) was performed in longitudinal sections of femurs from five-month-old female OVX mice treated with seAAV9 carrying amiR-ctrl or amiR-shn3. Serum CTX levels were assessed by ELISA (n=7) (FIG. 34C). Scale bar: 1 mm, (FIG. 34A); 50 mm, (FIG. 34B). Values represent mean±SD; NS, not significant, ***, P<0.001 and ****, P<0.0001 by an unpaired two-tailed Student's t-test (FIG. 34C).

FIGS. 35A-35C show the characterization of scAAV9. DSS vectors in vitro or in vivo. (FIG. 35A) A single dose of 1×10¹¹ genome copies of scAAV9-Egfp, seAAV9. DSS-588-Egfp or rAAV9. DSS-Nter-Egfp was i.a. injected into knce joints of two-month-old male mice and femurs were cryo-sectioned to identify EGFP-expressing cells two weeks post-injection. GP, growth plate; CB, cortical bone; BM, bone marrow. Scale bar: 500 μm, FIG. 35A. FIGS. 35B and 35C show two days after infection with scAAV9-Egfp, scAAV9. DSS-588-Egfp or scAAV9. DSS-Nter-Egfp, COBs were cultured under osteogenic conditions for six days. Osteoblast differentiation was assessed by ALP activity (FIG. 35B) and osteogenic gene expression (FIG. 35C), Values represent mean±SD: N.S, non-significant by one-way ANOVA. test (FIGS. 35B and 35C),

FIGS. 36A-36E show the tissue distribution of systematically delivered scAAV9. DSS-Nter in mice. A single dose of PBS or 4×10¹¹ genome copies of scAAV9, scAAV9. DSS-588, or scAAV9. DSS-Nter was i.v. injected into two-month-old male mice and EGFP expression was monitored using IVIS-100 optical imaging two weeks post-injection. EGFP expression in the whole body (FIG. 36A) and quantification of EGFP expression in dissected tissues (FIG. 36B) are displayed. Heart (FIG. 36C), liver (FIG. 36D), and lumbar vertebrae (FIG. 36E) were cryo-sectioned to identify EGFP-expressing cells. GP, growth plate: BM, bone marrow. Scale bar: 100 μm, (FIGS. 36C, 36D, and 36E). Values represent mean±SD: *, P<0.05; **, P<0.01 and ***, P<0.001 by one-way ANOVA test (FIG. 36B).

FIGS. 37A-37C show the therapeutic effects of systematically delivered scAAV9. DSS-Nter carrying amiR-shn3 in a mouse model of postmenopausal osteoporosis. Sham or OVX surgery was performed on three-month-old female mice and six weeks later, a single dose of PBS or 4×10¹¹ genome copies of scAAV9. DSS-Nter carrying amiR-ctrl or aniR-shn3 were i.v. injected. Seven weeks after injection, EGFP expression was monitored by IVIS-100 optical imaging (FIG. 37A). EGFP-expressing cells in the cryo-sectioned femurs were identified by fluorescence microscopy (FIG. 37B). Trabecular bone mass in lumbar vertebrae was assessed by microCT and quantification is displayed (n=7˜8/group) (FIG. 37C). Trabecular number per cubic millimeter (Tb.N), trabecular thickness (Tb.Th), and trabecular space (Tb.Sp). TB, trabecular bone; BM, bone marrow. Scale bar: 100 mm, panel b. Values represent mean±SD: N.S, non-significant; *, P<0.05; **, P<0.01; ***, P<0.001 by one-way ANOVA test (FIG. 37C).

### DETAILED DESCRIPTION

Aspects of the disclosure relate to compositions (e.g., isolated nucleic acids, rAAVs, etc.) that when delivered to a subject are effective for modulating bone metabolism, for example by promoting or inhibiting bone formation and/or promoting or inhibiting bone resorption. Accordingly, methods and compositions described by the disclosure are useful, in some embodiments, for the treatment of diseases and disorders associated with dysregulated bone metabolism, such as osteoporosis, inflammation-induced bone loss, bone loss induced by breast or prostate cancer metastasis, osteopetrosis, osteosarcoma, and heterotrophic ossification

### Isolated Nucleic Acids

Compositions and methods for delivering a transgene (e.g. an inhibitory RNA, such as an shRNA, miRNA, etc.) to a subject are provided in the disclosure. The compositions typically comprise an isolated nucleic acid encoding a transgene (e.g., a protein, an inhibitory nucleic acid, etc.) capable of modulating bone metabolism. For example, in some embodiments, a transgene reduces expression of a target protein, such as a target protein associated with promoting or inhibiting bone formation.

“Bone metabolism” generally refers to a biological process involving bone formation and/or bone resorption. In some embodiments, bone metabolism involves the formation of new bone as produced by osteoblasts (OBs) and differentiated osteocytes, and/or mature bone tissue being resorbed by osteoclasts (OCs). OBs arise from the bone marrow derived mesenchymal cells that ultimately differentiate terminally into osteocytes. OB (and osteocyte) functions or activities include but are not limited to bone formation, bone mineralization, and regulation of OC activity, Decreased bone mass has been observed to result from inhibition of OB and/or osteocyte function or activity. Increased bone mass has been observed to result from increased OB and/or osteocyte function or activity. OCs arise from bone marrow-derived monocytes and in some embodiments have been observed to be controlled by signals from OBs. OC functions include bone resorption. In some embodiments, decreased bone mass has been observed to result from increased OC activity. In some embodiments, increased bone mass has been observed to result from inhibition of OC activity.

In some embodiments, an isolated nucleic acid or an rAAV as described by the disclosure comprises a transgene encoding at least one bone metabolism modulating agent (e.g., 1, 2, 3, 4, 5, 6, 7, 8, 9, 10, or more bone metabolism modulating agents). As used herein, a “bone metabolism modulating agent” refers to a molecule (a nucleic acid or protein encoded by a nucleic acid, e.g., a transgene) that either induces or inhibits bone formation or deposition, for example by increasing or decreasing expression, activity, and/or function of proteins, cells, etc., that are involved in bone formation or bone resorption. Generally, a bone metabolism modulating agent can be a peptide, protein, or an interfering nucleic acid (e.g., dsRNA, siRNA, shRNA, miRNA, artificial miRNA, etc.). In some embodiments, a bone metabolism modulating agent is a bone formation inducing agent. In some embodiments, a bone metabolism modulating agent is a bone formation inhibiting agent.

A “bone formation inducing agent” refers to a molecule that promotes bone synthesis either by promoting OB and/or osteocyte (OCY) differentiation or activity and/or by inhibiting OC activity. In some embodiments, a bone formation inducing agent is a nucleic acid (e.g., RNAi oligonucleotide or miRNA oligonucleotide or antisense oligonucleotide) or protein encoded by a nucleic acid (e.g., a transgene) that promotes OB and/or osteocyte function or activity (e.g., bone formation, mineralization, regulation of osteoclast activity or function, etc.). In some embodiments, examples of bone formation inducing agents that promote OB and/or osteocyte activity or function include but are not limited to parathyroid hormone (PTH), PTH-related protein (PTHrP), deglycase DJ1. In some embodiments, a bone formation inducing agent is an inhibitory nucleic acid that inhibits OC differentiation or activity, such as an inhibitory nucleic acid that targets selerostin (SOST), schnurri-3 (SHN3), cathepsin K (CTSK), etc.

A “bone formation inhibiting agent” refers to a molecule that inhibits OB and/or osteocyte differentiation or activity and/or increases OC differentiation, activity or function. In some embodiments, a bone formation inhibiting agent is a nucleic acid (e.g., RNAi oligonucleotide or miRNA oligonucleotide or antisense oligonucleotide) or protein encoded by a nucleic acid (e.g., a transgene) that inhibits OB and/or osteocyte differentiation or activity. In some embodiments, examples of bone formation inhibiting agents that inhibit OB and/or osteocyte activity or function include but are not limited to a MAPK inhibitor, and pro-inflammatory cytokines (e.g., tumor necrosis factor alpha (TNF-α), etc. In some embodiments, a bone formation inhibiting agent is an inhibitory nucleic acid that inhibits OB and/or osteocyte differentiation or activity.

In some embodiments, an isolated nucleic acid encodes one or more (e.g., 1, 2, 3, 4, 5, 6, 7, 8, 9, 10, or more) inhibitory nucleic acids, for example dsRNA, siRNA, shRNA, miRNA, artificial microRNA (ami-RNA), etc.). Generally, an inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a gene encoding a gene product (e.g., a protein) associated with bone metabolism (e.g., PTH, PTHrP, DJ1, SOST, SHN3, CTSK, etc.). As used herein “continuous bases” refers to two or more nucleotide bases that are covalently bound (e.g., by one or more phosphodiester bond, etc.) to each other (e.g. as part of a nucleic acid molecule). In some embodiments, the at least one inhibitory nucleic acid is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of a gene encoding a gene product (e.g., a protein) associated with bone metabolism (e.g., PTH, PTHrP. DJ1, SOST, SHN3, CTSK, RANK, etc.).

A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g. passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a bone metabolism modulating (e.g., bone formation inhibiting agent) miRNA has been inserted in place of the endogenous miR-155 mature miRNA-encoding sequence. In some embodiments, miRNA (e.g., an artificial miRNA) as described by the disclosure comprises a miR-155 backbone sequence, a miR-30 backbone sequence, a mir-64backbone sequence, or a miR-122 backbone sequence.

In some embodiments, the present disclosure provides an isolated nucleic acid comprising a transgene encoding an artificial microRNA targeting the SHIN3 gene (GeneID:

59269). which encodes the Schnurri-3 protein. The Schnurri-3 (SHN3) protein is a transcription factor that regulates NK-β protein expression and immunoglobulin and T-cell. receptor antibody recombination. In some embodiments, the SHN3 gene is represented by the NCBI Accession Number NM_001127714.2 or NM_024503.5. In some embodiments, the SHN3 protein is represented by the NCBI Accession Number NP_001121186.1 or NP_078779.2.

In some aspects, the disclosure relates to an isolated nucleic acid comprising a transgene encoding an artificial microRNA is used to reduce SHN3 expression (e.g., expression of one or more gene products from an SHN3 gene, for example an mRNA encoded by the nucleotide sequence in SEQ ID NO: 69 or the encodes a protein having the amino acid sequence set forth in SEQ ID NO: 70.

In some embodiments, an artificial microRNA targets (e.g., binds to, or comprises a region of complementarity with) at least 6 continuous nucleotides of a SHN3 gene. In some embodiments, an artificial microRNA targets (e.g., binds to, or comprises a region of complementarity with) between 6 and 30 continuous nucleotides of a SHN3 gene. In some embodiments, an artificial microRNA targets between 12-24 continuous nucleotides of a SHN3 gene. In some embodiments, an artificial microRNA targets between 9-27 continuous nucleotides of the SHIN3 gene. In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a SHN3 gene.

In some embodiments, an artificial microRNA is between 6-50 nucleotides in length. In some embodiments, an artificial microRNA is between 8-24 nucleotides in length. In some embodiments, an artificial microRNA is between 12-36 nucleotides in length. In some embodiments, an artificial microRNA is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an isolated inhibitory nucleic acid decreases expression of a target gene by between 50% and 99% (e.g., any integer between 50% and 99%, inclusive). In some aspects, an isolated inhibitory nucleic acid decreases expression of a target gene by between 75% and 90%. In some aspects, an isolated inhibitory nucleic acid decreases expression of a target gene by between 80% and 99%. In some embodiments, an isolated inhibitory nucleic acid decreases expression of a SHN3 gene by between 50% and 99% (e.g., any integer between 50% and 99%, inclusive). In some embodiments, an isolated inhibitory nucleic acid decreases expression of a SHN3 gene by between 75% and 90%. In some aspects, an isolated inhibitory nucleic acid decreases expression of a SHN3 gene by between 80% and 99%.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired.

sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A. rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler. S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy. 1999; 6:198-208; de Felipe, P et al., Human Gene Therapy, 2000; 11:1921-1931.; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530(1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systemis have been described and can be readily selected by one of skill in the art. Examples of inducible promoters regulated by exogenonsly supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551(1992)), the tetracycline-inducible system (Gossen et al., Science, 268:1766-1769 (1995), see also Harvey et al., Carr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimie the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther . . . 3:1002-9 (1996); alpha-fetoprotein (APP) promoter. Arbuthnot et al., Hum. Gene Ther., 7:1503-14 (1996), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15(1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, a tissue-specific promoter is a bone tissue-specific promoter. Examples of bone tissue-specific promoters include but are not limited to promoters of osterix, osteocalcin, type 1 collagen al, DMP1, cathepsin K, Rank, etc.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence, Non-limiting examples of polIII promoter sequences include U6 and HI promoter sequences. In some embodiments, the second promoter sequence (e.g., the promoter sequence driving expression of the inhibitory RNA) is a RNA polymerase II (poIII) promoter sequence. Non-limiting examples of poIII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polli promoter sequence drives expression of a protein coding region.

### Recombinant AAVs (rAAVs)

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and S′ and 3′ AAV inverted terminal repeats (ITRs). The transgene may comprise, as disclosed elsewhere herein, one or more regions that encode one or more proteins and/or inhibitory nucleic acids (e.g., shRNA, miRNAs, etc.) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences, The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAVI, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, seAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and AmiRNA) can serve a function similar to a mutant inverted terminal repeat (mTR) during viral genome replication, generating self-complementary AAV vector genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, ami-RNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ ITR. In some embodiments, the ITR is a 3′ ITR Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol. 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3. AAV4, AAV5, AAV6, AAV7, AAV8, AAVrh8, AAV9, AAV10, and/or AAVrh10 ITR sequence.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g. AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of. AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAVI, AAV2, AAV3, AAV4, AAVS, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.xh39, or AAV.rh43 serotype. In some embodiments, an AAV capsid protein is of an AAV9 serotype.

The disclosure is based, in part, on rAAVs comprising capsid proteins that have increased tropism for bone tissue. In some embodiments, the capsid proteins are grafted to a bone-targeting peptide. A heterologous bone-targeting peptide may target OCs (e.g., specifically, or preferentially targets OCs relative to OBs) or OBs (e.g., specifically, or preferentially targets OBs relative to OCs). In some embodiments, a bone-targeting peptide is an (AspSerSer)₆ peptide, which may also be referred to as a DSS peptide (e.g. SEQ ID NO: 16). Additional bone-targeting peptide is a HABP-19 peptide (CγEPRRγEVAYELγEPRRγEVAγEL; SEQ ID NO: 17), which may also be referred to as a HABP peptide. In some embodiments, a bone-targeting peptide is an (Asp)₈₋₁₄ peptide comprising 8-14 aspartic acid residues (e.g., as set forth in SEQ ID NOs: 57-63). Further examples of bone-targeting peptides include but are not limited to those described by Ouyang et al. (2009) Lett. Organic Chem 6 (4): 272-277. In some embodiments, bone-targeting peptides comprise the sequence set forth in SEQ ID NO: 16,17, 57, 58, 59, 60, 61, 62, and 63.

As used herein, “grafting” refers to joining or uniting of one molecule with another molecule. In some embodiments, the term grafting refers to joining or uniting of at least two molecules such that one of the at least two molecules is inserted within another of at least two molecules. In some embodiments, the term grafting refers to joining or uniting of at least two polymeric molecules such that one of at least two molecules is appended to another of at least two molecules. In some embodiments, the term grafting refers to joining or uniting of one polymeric molecule (e.g., a nucleic acid, a polypeptide) with another polymeric molecule (e.g., a nucleic acid, a polypeptide). In some embodiments, the term grafting refers to joining or uniting of at least two nucleic acid molecules such that one of at least two molecules is appended to another of at least two nucleic acid molecules.

In some embodiments, the term grafting refers to joining or uniting of at least two nucleic acid molecules such that one of the at least two nucleic acid molecules is inserted within another of the at least two nucleic acid molecules. For example, it has been observed that targeting peptides may be grafted to certain loci of a nucleic acid encoding a VP2 AAV capsid protein. In some embodiments, a targeting peptide (e.g. a bone-targeting peptide) is inserted at a position corresponding to the position between the codons encoding Q588 and A589 and/or N587 and R588 of an AAV2 or AAV9 VP2 capsid protein. In some embodiments, a targeting peptide is inserted at a position between the codons encoding N587 and R588 of an VP3 capsid protein (or a position corresponding to such amino acid positions in AAV2 or AAV9). In some embodiments, a targeting peptide is inserted at a position between the codons encoding S452 and G453 of an VP1 capsid protein. Other potential positions may be N587 and R588.

In some embodiments, a nucleic acid formed through grafting (a grafted nucleic acid) encodes a chimeric protein. In some embodiments, a grafted nucleic acid encodes a chimeric protein, such that one polypeptide is effectively inserted into another polypeptide (e.g. not directly conjugated before the N-terminus or after the C-terminus), thereby creating a contiguous fusion of two polypeptides. In some embodiments, a grafted nucleic acid encodes a chimeric protein, such that one polypeptide is effectively appended to another polypeptide (e.g. directly conjugated before the N-terminus or after the C-terminus), thereby creating a contiguous fusion of two polypeptides. In some embodiments, the term grafting refers to joining or uniting of at least two polypeptides, or fragments thereof, such that one of the at least two polypeptides or fragments thereof is inserted within another of the at least two polypeptides or fragments thereof. In some embodiments, the term grafting refers to joining or uniting of at least two polypeptides or fragments thereof such that one of the at least two polypeptides or fragments thereof is appended to another of the at least two polypeptides or fragments thereof.

In some embodiments, the disclosure relates to an adeno-associated virus (AAV) capsid protein that is conjugated to one or more bone-targeting moieties. A “bone-targeting moiety” generally refers to a small molecule, peptide, nucleic acid, etc., that facilitates trafficking of an rAAV to bone or bone tissue. For example, in some embodiments, a bone-targeting moiety is a peptide or small molecule that binds to a receptor on a bone cell (e.g., OB, OC, osteocyte, etc.). Examples of bone-targeting moieties include but are not limited to alendronate (ALE), polypeptides such as cyclic arginine-glycine-aspartic acid-tyrosine-lysine (cRGCyk), Asp-Asp-Asp-Asp-Asp-Asp-Asp-Asp (D-Asp8), and aptamers such as CH6. A bone-targeting moiety may be conjugated directly to a capsid protein or conjugated to a capsid protein via a linker molecule (e.g., an amino acid linker, a PEG linker, etc.).

In some embodiments, a linker is a glycine-rich linker. In some embodiments, a linker comprises at least two glycine residues. In some embodiments, a linker comprises GGGGS (SEQ ID NO: 64). In some embodiments, the linker comprises a formula selected from the group consisting of: [G]ₙ (SEQ ID NO: 65), [G]ₙS (SEQ ID NO: 66), [GS]ₙ (SEQ ID NO: 67), and [GGSG]ₙ (SEQ ID NO: 68), wherein G is glycine and wherein n is an integer greater than one (e.g., 2, 3, 4, 5, 6, 7, 8, 9, 10, 11, 12, 13, 14, 15, 16, 17, 18, 19, 20, 21, 22, 23, 24, 25 or more). In some embodiments, n is an integer in a range of 2 to 10, 2 to 20, 5 to 10, 5 to 15, or 5 to 25. Accordingly, in some embodiments, a heterologous targeting peptide is conjugated to a linker.

In some embodiments, a capsid protein comprises one or more azide-bearing unnatural amino acids which are capable of reacting with an ADIBO-tagged bone-targeting moiety (e.g., via “click chemistry” to form a capsid protein-bone-targeting moiety conjugate. Capsid proteins comprising unnatural azide-bearing amino acids are described, for example by Zhang et al. (2016) Biomaterials 80:134-145, and use of ADIBO-based click chemistry for peptide conjugation is described, for example by Prim et al. (2013) Molecules 18 (8): 9833-49. The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions ander the control of a constitutive promoter). but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV belper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (Le., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (Sf9), or a mammalian (e.g., human, rodent, non-human primate, etc.) cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomie or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In some aspects, the present disclosure provides a recombinant AAV comprising a capsid protein and an isolated nucleic acid comprising a first region encoding an AAV ITR and a second region comprising a transgene, wherein the transgene encodes an artificial microRNA. The artificial microRNA may decrease the expression of a target gene in a cell (e.g. osteoblasts, osteoclasts, osteocytes, chondrocytes) or a subject. In some embodiments, the rAAV comprises an artificial microRNA that decreases the expression of SHN3 in a cell or a subject.

The rAAV may comprise at least one modification which increases targeting of the rAAV to bone cells (e.g., osteoblasts, osteoclasts, osteocytes, chondrocytes). Non-limiting examples of modifications which increase targeting of the rAAV to bone cells include heterologous bone-targeting peptides (e.g., as set forth in any one of SEQ ID NOs: 16,17, 57, 58, 59, 60, 61, 62, or 63), AAV capsid serotypes (e.g., AAV1, AAV4, AAV5, AAV6, AAV6.2, AAV7, AAV8, AAV9, AAV10, AAVrh39, AAVrh43).

Expression of SHN3 in a cell or subject may be decreased by between 50% and 99% (e.g., any integer between 50% and 99%, inclusive) using rAAVs of the present disclosure. Expression of SHN3 in a cell or subject may be decreased by between 75% and 90% using rAAVs of the present disclosure. Expression of SHIN3 in a cell or subject may be decreased by between 80% and 99% using rAAVs of the present disclosure.

The foregoing methods for packaging recombinant vectors in desired AAV capsids to produce the rAAVs of the disclosure are not meant to be limiting and other suitable methods will be apparent to the skilled artisan.

### Modes of Administration and Compositions

The rAAVs of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig. guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the bone (e.g., bone tissue) of a subject. By “bone tissue” is meant all cells and tissue of the bone and/or joint (e.g., cartilage, axial and appendicular bone, etc.) of a vertebrate. Thus, the term includes, but is not limited to, osteoblasts, osteocytes, osteoclasts, chondrocytes, and the like. Recombinant AAVs may be delivered directly to the bone by injection into, e.g., directly into the bone, via intrasynovial injection, knee injection, femoral intramedullary injection, etc., with a needle, catheter or related device, using surgical techniques known in the art. In some embodiments, rAAV as described in the disclosure are administered by intravenous injection. In some embodiments, the rAAV are administered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more bone metabolism modulating agents. In some embodiments, the nucleic acid further comprises one or more AAV ITRs. In some embodiments, the rAAV comprises an rAAV vector comprising the sequence set forth in any one of SEQ ID NO: 1-15 (or the complementary sequence thereof), or a portion thereof, In some embodiments, a composition further comprises a pharmaceutically acceptable carrier.

In some embodiments, compositions comprise a recombinant AAV comprising a capsid protein and a nucleic acid comprising a first region encoding an AAV ITR and a second region comprising a transgene, wherein the transgene encodes an artificial microRNA that targets SHN3. In some embodiments, the recombinant AAV comprises a sequence as set forth in SEQ ID NO: 3. In some embodiments, the capsid protein further comprises a heterologous bone-targeting peptide as set forth in SEQ ID NOs: 16-17 or 57-63.

Aspects of the disclosure provide a method of decreasing SHN3 expression in a cell. A cell may be a single cell or a population of cells (e.g., culture). A cell may be in vivo (e.g., in a subject) or in vitro (e.g., in culture). A subject may be a mammal, optionally a human, a mouse, a rat, a non-human primate, a pig, a dog, a cat, a chicken, or a cow.

Expression of SHN3 in a cell or subject may be decreased by between 50% and 99% (e.g., any integer between 50% and 99%, inclusive) using isolated nucleic acids, rAAVs, or compositions of the present disclosure. Expression of SHN3 in a cell or subject may be decreased by between 75% and 90% using isolated nucleic acids, rAAVs, or compositions of the present disclosure. Expression of SHN3 in a cell or subject may be decreased by between 80% and 99% using isolated nucleic acids, rAAVs, or compositions of the present disclosure.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes,

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An “effective amount” of an rAAV is an amount sufficient to target infect an animal, target a desired tissue (e.g., bone tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target bone tissue.

In some embodiments, a dose of rAAV is administered to a subject no more than once per calendar day (e.g., a 24-hour period). In some embodiments, a dose of rAAV is administered to a subject no more than once per 2, 3, 4, 5. 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments. rAAV compositions are formulated to reduce aggregation of AAV particles in the composition, particularly where high rAAV concentrations are present (e.g.,˜1013 GC/ml or more). Methods for reducing aggregation of rAAVs are well known in the art and, include, for example, addition of surfactants, pH adjustment, salt concentration adjustment, etc. (See, e.g., Wright FR, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, femoral intramedullary, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants, The prevention of the action of microorganisms can be brought about by various antibacterial and antifungal agents, for example, parabens, chlorobutanol, phenol, sorbic acid, thimerosal, and the like, In many cases, it will be preferable to include isotonic agents, for example, sugars or sodium chloride, Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells, In particular, the rAAV vector delivered transgenes may be formulated for delivery either encapsulated in a lipid particle, a liposome, a vesicle, a nanosphere, or a nanoparticle or the like. Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587),

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agente, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

Methods for delivering an effective amount of a transgene (e.g., an isolated nucleic acid or rAAV encoding one or more bone metabolism modulating agent) to a subject are provided by the disclosure. In some embodiments, the methods comprise the step of administering to a subject an effective amount of an isolated nucleic acid encoding an interfering RNA capable of promoting or inhibiting bone formation. In some embodiments, the methods comprise the step of administering to a subject an effective amount of an isolated nucleic acid encoding an interfering RNA capable of promoting or inhibiting bone resorption. Thus, in some embodiments, isolated nucleic acids, rAAVs, and compositions described herein are useful for treating a subject having or suspected of having a disease or disorder associated with dysregulated bone metabolism.

As used herein, a “disease or disorder associated with dysregulated bone metabolism” refers to a condition characterized by an imbalance between bone deposition and bone resorption resulting in either 1) abnormally increased bone deposition (e.g., formation) relative to a healthy individual (e.g., a subject not having a disease characterized by imbalance between bone deposition and bone resorption), or 2) abnormally decreased bone deposition (e.g., formation) relative to a healthy individual (e.g., a subject not having a disease characterized by an imbalance between bone deposition and bone resorption), or 3) abnormally increased bone resorption (e.g., breakdown) relative to a healthy individual (e.g., a subject not having a disease characterized by imbalance between bone deposition and bone resorption), or 4) abnormally decreased bone resorption (e.g., breakdown) relative to a healthy individual (e.g., a subject not having a disease characterized by imbalance between bone deposition and bone resorption). A “disease associated with reduced bone density” refers to a condition characterized by increased bone porosity resulting from either 1) abnormally decreased bone deposition (e.g., formation) relative to a healthy individual (e.g., a subject not having a disease characterized by decreased bone density), or 2) abnormally increased bone resorption (e.g., breakdown) relative to a healthy individual (e.g., a subject not having a disease characterized by decreased bone density). A disease associated with increased bone porosity may arise from either 1) abnormally decreased OB and/or osteocyte differentiation, function, or activity relative to a healthy individual (e.g., a subject not having a disease characterized by decreased bone density) and/or 2) abnormally increased OC differentiation, function, or activity relative to a healthy individual (e.g., a subject not having a disease characterized by decreased bone density). “Porosity” generally refers to the volume of fraction of bone not occupied by bone tissue.

A “disease associated with increased bone density” refers to a condition characterized by decreased bone porosity resulting from either 1) abnormally increased bone deposition (e.g., formation) relative to a healthy individual (e.g., a subject not having a disease characterized by increased bone density), or 2) abnormally decreased bone resorption (e.g., breakdown) relative to a healthy individual (e.g., a subject not having a disease characterized by increased bone density). A disease associated with decreased bone porosity may arise from either 1) abnormally increased OB and/or osteocyte differentiation, function, or activity relative to a healthy individual (e.g., a subject not having a disease characterized by increased bone density) and/or 2) abnormally decreased OC differentiation, function, or activity relative to a healthy individual (e.g., a subject not having a disease characterized by increased bone density).

Aspects of the present disclosure provide methods of treating a disease or disorder associated with dysregulated bone metabolism. Dysregulated bone metabolism may be diseases associated with reduced bone density (e.g., osteoporosis, critical sized-bone defects, a mechanical disorder resulting from disuse or injury). Dysregulated bone metabolism may be diseases associated with increased bone density (e.g., osteopetrosis, pycnodysostosis, sclerosteosis, acromegaly, fluorosis, myelofibrosis, hepatitis C-associated osteosclerosis, heterotrophic ossification).

In some embodiments, methods of treating a disease or disorder associated with a dysregulated bone metabolism comprise administering to a subject in need thereof a recombinant AAV (rAAV) comprising a transgene. A rAAV may comprise a modification that promotes its targeting to bone cells (e.g., osteoclasts and osteoblasts). Non-limiting modifications of rAAVs that promote its targeting to bone cells include the heterologous bone-targeting peptide in SEQ ID NOs: 16-17 or 57-63, bone-specific promoters, and AAV serotypes with increased targeting to bone relative to other tissues. In some embodiments, methods of treating a dysregulated bone metabolism comprise administering to a subject in need thereof a rAAV comprising a heterologous bone-targeting peptide as in SEQ ID NOs: 16-17 or 57-63.

In some embodiments, the rAAV comprising the heterologous bone-targeting peptide comprises a transgene which upregulates or downregulates a target gene associated with dysregulation of bone metabolism. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density.

Aspects of the disclosure provide methods for treating a disease or disorder associated with a disease of disorder characterized by dysregulation of bone metabolism comprising administering to a subject a rAAV comprising a capsid protein and an isolated nucleic acid encoding an inhibitory nucleic acid. The rAAV may comprise an inhibitory nucleic acid (e.g., siRNA, shRNA, miRNA, or amiRNA). The inhibitory nucleic acid may decrease or increase expression of a target gene associated with a disease or disorder characterized by dysregulation of bone metabolism.

In some embodiments, the present disclosure provides a method of treating disease or disorder associated with reduced bone density. The method comprises administering to a subject in need thereof a rAAV or an isolated nucleic acid comprising a transgene that targets a gene associated with reduced bone density. In some embodiments, the rAAV or isolated nucleic acid comprises a transgene encoding an artificial microRNA that targets a gene associated with reduced bone density. In some embodiments, the target gene is SHIN3, PTH, PTHrP, DJ1, SOST, CTSK, or RANK.

Aspects of the present disclosure provide methods of treating a disease or disorder associated with reduced bone density comprising administering an rAAV or an isolated nucleic acid. In some embodiments, the rAAV or isolated nucleic acid comprise a sequence as set forth in SEQ ID NO: 3, or the complement thereof. In some embodiments, the rAAV comprises a capsid protein comprising a sequence as set forth in any one of SEQ ID NOs: 18-34. In some embodiments, the rAAV comprises a heterologous bone-targeting peptide comprising a sequence as set forth in any one of SEQ ID NOs: 16-17 and 57-63.

Expression of SHN3 in a cell or subject may be decreased by between 50% and 99% (e.g., any integer between 50% and 99%, inclusive) using methods of the present disclosure. Expression of SHN3 in a cell or subject may be decreased by between 75% and 90% using methods of the present disclosure. Expression of SHN3 in a cell or subject may be decreased by between 80% and 99% using methods of the present disclosure.

In some embodiments, an “effective amount” of a substance is an amount sufficient to produce a desired effect (e.g., to transduce bone cells or bone tissue). In some embodiments, an effective amount of an isolated nucleic acid is an amount sufficient to transfect (or infect in the context of rAAV-mediated delivery) a sufficient number of target cells of a target tissue of a subject. In some embodiments, a target tissue is bone tissue (e.g., bone and bone tissue cells, such as OBs, OCs, osteocytes, chondrocytes, etc.). In some embodiments, an effective amount of an isolated nucleic acid (e.g., which may be delivered via an rAAV) may be an amount sufficient to have a therapeutic benefit in a subject, e.g., to increase activity or function of OBs and/or osteocytes, to inhibit activity of OBs and/or osteocytes, to increase activity of function of OCs, to inhibit activity or function of OCs, etc. The effective amount will depend on a variety of factors such as, for example, the species, age, weight, health of the subject, and the tissue to be targeted, and may thus vary among subject and tissue as described elsewhere in the disclosure.

Exemplary embodiments of the invention will be described in more detail by the following examples. These embodiments are exemplary of the invention, which one skilled in the art will recognize is not limited to the exemplary embodiments.

### EXAMPLES

### Example 1

Several key regulators of bone formation have been identified as therapeutic targets to osteoporosis. For instance, suppression of naturally occurring inhibitors of bone formation, sclerostin (SOST) or the adaptor protein schnurri-3 (SHN3, also HIVEP3) results in a progressive increase in bone mass due to augmented OB activity. Unlike SHN3 and SOST that primarily function in OBs, cathepsin K (CTSK) is highly expressed in OCs and its inhibition increases bone mass by blocking OC activity. Lastly, treatment with bone anabolic factors, including PTH, PTH-related protein (PTHrP), or DJ-1 promotes bone formation. In this example, gene therapeutics that can manipulate expression of these candidate genes using bone-targeting adeno-associated virus (AAV)-mediated gene silencing or addition are described. In some embodiments, therapeutics (e.g., compositions described by the disclosure) prevent bone loss by promoting OB function and bone formation or by suppressing OC activity and bone destruction with limited adverse effects on bone remodeling and regeneration activity.

### Development of Gene Therapeutics Using Bone-Targeting Adeno-Associated Viruses (BT-AAVs)

The high efficiency of transduction, persistent transgene expression, and lack of post-infection immunogenicity and pathogenicity make AAVs very attractive viral vectors for use in gene therapy. To date, AAV vectors have been evaluated in over 130 clinical trials and 2,000 patients worldwide. However, non-specific cell targeting, possible pre-existing immunity, and other rate-limiting events necessitate identification of more natural AAV serotypes and. improvement of the properties and functions of AAV serotypes by vector engineering. Here, AAV serotypes that can transduce OBs and/or OCs in vitro and in vivo were identified.

### Identification of AAV Serotypes that Transduce OBs, OCs, and Chondrocytes

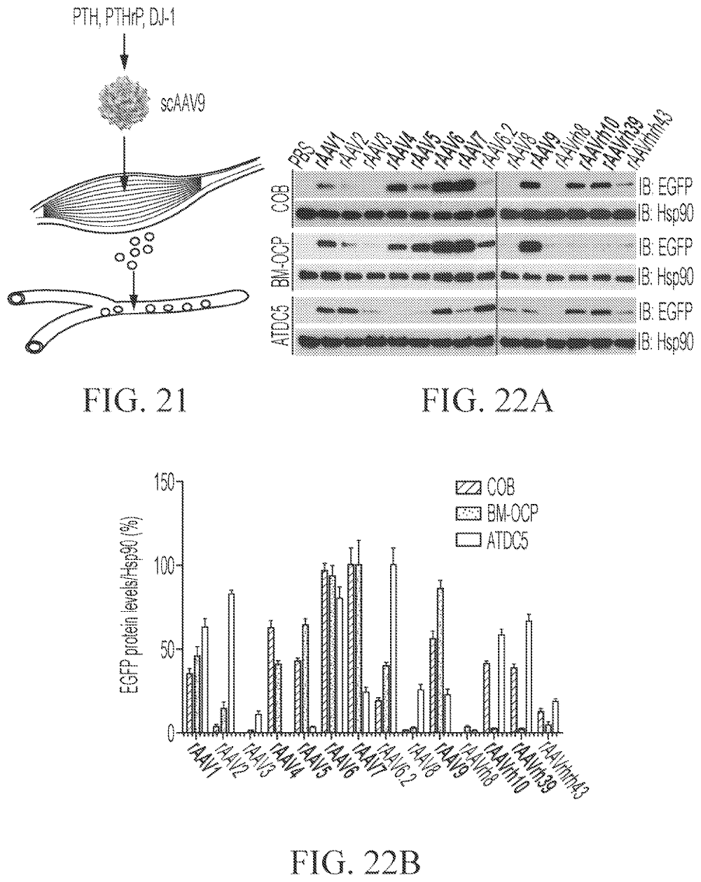

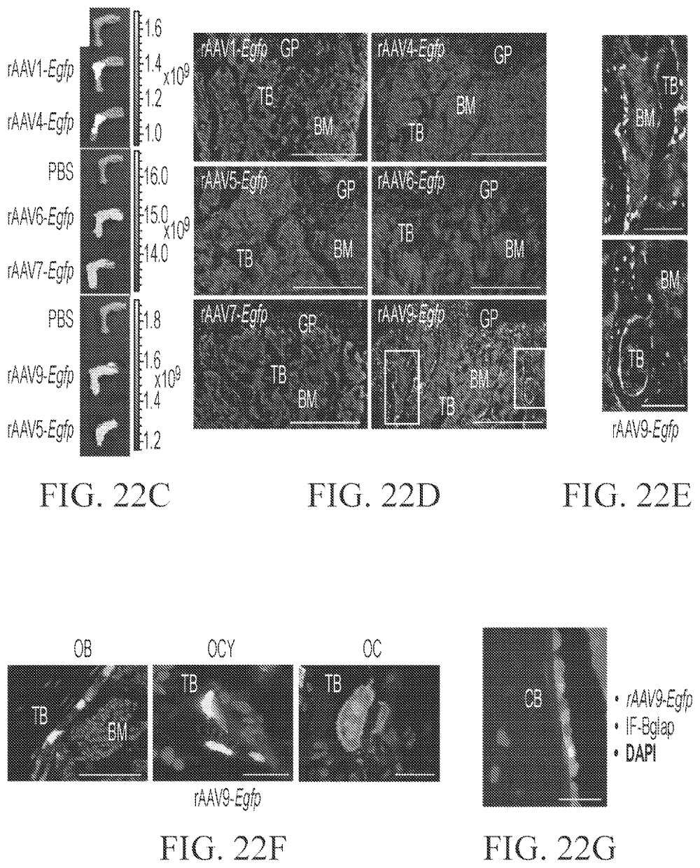

To this end, enhanced green fluorescent protein (GFP) reporter gene was packaged into 17 AAV capsids (scAAVI, scAAV2, scAAV3, scAAV4, scAAV5, scAAV6, scAAV6.2, scAAV7, scAAV8, scAAV9, scAAVrh8, scAAVrh10, scAAVrh39, scAAVrh43, scAAV2/2-66, scAAV2/2-84, scAAV2/2-125). The ability of these purified AAV serotype vectors to transduce mouse OB-or chondrocyte-lineage cells originated from mesenchymal stem cells (MSCs) or mouse OCs originated from bone marrow monocytes (BM-MO) were assessed by monitoring GFP expression using epifluorescence microscopy. First, to test their transduction capability to OB-or chondrocyte-lineage cells, mouse MSCs and chondrocyte progenitor line (ATDC5) were purchased from Cyagen and ATCC, respectively. Additionally, mouse OB precursors (pre-OB) were isolated from the calvaria of mouse neonates at postnatal day 3-5 on three different congenic backgrounds (C57BL/6J, BALB/cJ, 129S1/SvimJ), and cultured in osteogenic medium for 6 days to differentiate into mature OBs (mOB). A single dose of 10¹²˜10¹³/ml genome copies of 17 purified AAV serotypes were incubated with these cells for 2 days and GFP expression was analyzed by epifluorescence microscopy (Table 1) and immunoblotting against EGFP (FIG. 22).

TABLE 1
Identification of scAAV serotypes that can transduce mouse MSC-lineage cells in vitro.

| | | | Pre-OB | Pre-OB | Pre-OB | mOB | mOB | | |
|---|---|---|---|---|---|---|---|---|---|
| | AAV serotypes | Titer | (B6) | (129) | (Balbc) | (129) | (Balbc) | MSC | ATDC5 |
| 1 | scAAV1.CB6- | 1.8 × 10¹³ | + | + | ++ | + | ++ | + | + |
| | eGFP | GC/mL | | | | | | | |
| 2 | scAAV2.CB6- | 1.5 × 10¹² | − | − | − | − | − | − | − |
| | eGFP | GC/mL | | | | | | | |
| 3 | scAAV3.CB6- | 6 × 10¹³ | − | − | − | − | − | − | − |
| | eGFP | GC/mL | | | | | | | |
| 4 | scAAV4.CB6- | 6.5 × 10¹² | +++ | ++ | ++ | ++ | ++ | + | − |
| | eGFP | GC/mL | | | | | | | |
| 5 | scAAV5.CB6- | 1.4 × 10¹³ | + | + | ++ | + | ++ | ++ | ++ |
| | eGFP | GC/mL | | | | | | | |
| 6 | scAAV6.CB6- | 8 × 10¹² | ++ | + | ++ | + | ++ | + | ++ |
| | eGFP | GC/mL | | | | | | | |
| 7 | scAAV6.2.CB6- | 8 × 10¹² | ++ | + | + | ++ | ++ | + | ++ |
| | eGFP | GC/mL | | | | | | | |
| 8 | scAAV7.CB6- | 1.5 × 10¹³ | + | − | + | − | +/− | + | − |
| | eGFP | GC/mL | | | | | | | |
| 9 | scAAV8.CB6- | 7 × 10¹² | − | + | − | − | +/− | − | + |
| | eGFP | GC/mL | | | | | | | |
| 10 | scAAV9.CB6- | 1.5 × 10¹³ | ++ | − | − | +/− | +/− | − | − |
| | eGFP | GC/mL | | | | | | | |
| 11 | scAAVrh8.CB6- | 8 × 10¹² | − | − | − | − | − | − | − |
| | eGFP | GC/mL | | | | | | | |
| 12 | scAAV10.CB6- | 8 × 10¹² | − | − | − | − | +/− | − | − |
| | eGFP | GC/mL | | | | | | | |
| 13 | scAAVrh39.CB6- | 1 × 10¹³ | + | − | − | +/− | +/− | − | − |
| | eGFP | GC/mL | | | | | | | |
| 14 | scAAVrh43.CB6- | 6 × 10¹² | − | − | − | − | − | − | +/− |
| | eGFP | GC/mL | | | | | | | |
| 15 | scAAV2/2-66.CB6- | 1 × 10¹³ | ND | − | − | − | − | ND | +/− |
| | eGFP | GC/mL | | | | | | | |
| 16 | scAAV2/2-84.CB6- | 4 × 10¹² | ND | − | − | − | − | ND | +/− |
| | eGFP | GC/mL | | | | | | | |
| 17 | scAAV2/2-125.CB6- | 5 × 10¹² | ND | − | − | − | − | ND | +/− |
| | eGFP | GC/mL | | | | | | | |

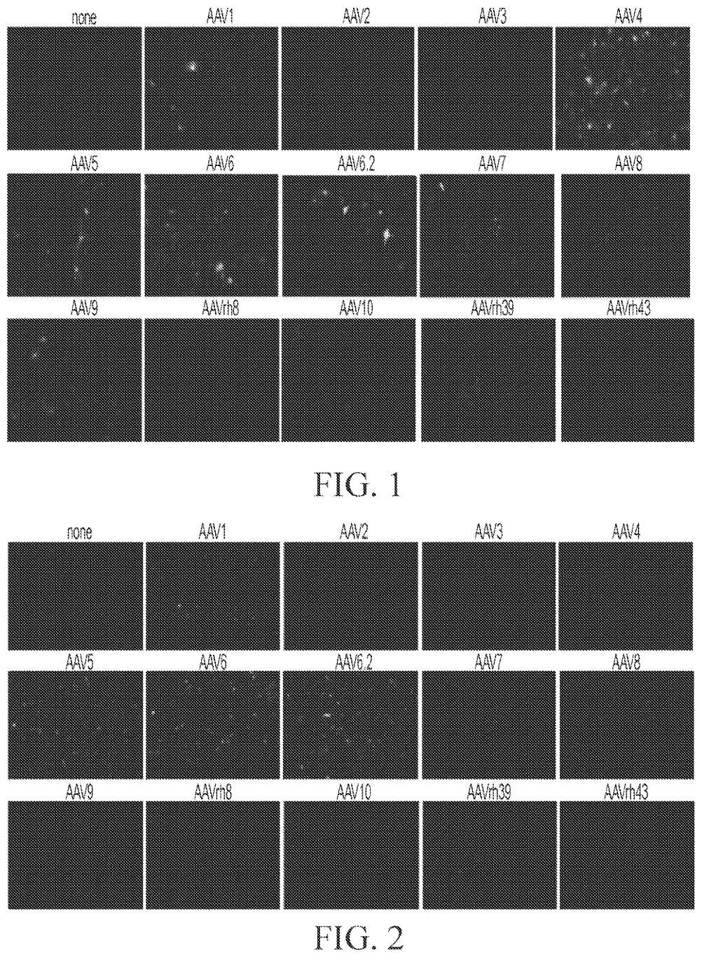

Among 17 AAV serotypes, scAAV4 was most effective to transduce mouse OB-lineage cells on three different mouse backgrounds, scAAVI, scAAV5, scAAV6, and scAAV6.2(engineered version of seAAV6) transduced both mouse OB and chondrocyte-lineage cells on three different mouse backgrounds, and scAAV9 transduced only pre-OBs on the C57B6/J background (representative images in FIG. 1 and FIG. 2).

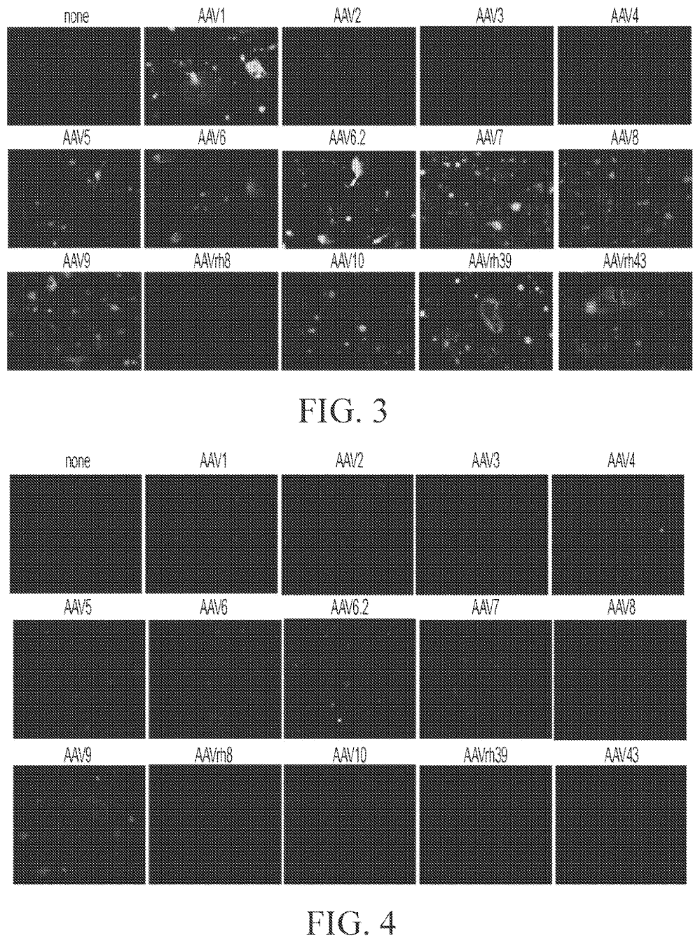

Next. the ability of purified AAV serotypes to transduce mouse OC precursors was investigated. Mouse primary OC precursors (BM-MO, bone-marrow derived monocyte) were isolated from the long bones of 2-month old mice (C57BL/6J) and amplified by the addition of mouse M-CSF (40 ng/ml, R&D systems). BM-MOs were cultured in the presence of mouse M-CSF (40 ng/ml) and mouse Rank ligand (10 ng/ml, R&D systems) for 6 days to differentiate into mature OCs (FIG. 3). Additionally, mouse OC precursor line (Raw264.7) was differentiated into mature OCs 4 days after culture with mouse Rank ligand (10 ng/ml) (FIG. 4). A single dose of 10¹²˜10¹³/ml genome copies of 17 purified AAV serotypes were incubated with these cells for 2 days and GFP expression was analyzed by epifluorescence microscopy (Table 2).

TABLE 2
Identification of scAAV serotypes that transduce mouse OCs in vitro.

| AAV | serotypes | Titer | BM-MO (B6) | BM-OC (B6) | Raw-OC |
|---|---|---|---|---|---|
| 1 | scAAV1.CB6-eGFP | 1.8 × 10¹³ GC/mL | − | + | ++ |
| 2 | scAAV2.CB6-eGFP | 1.5 × 10¹² GC/mL | − | + | − |
| 3 | scAAV3.CB6-eGFP | 6 × 10¹³ GC/mL | − | − | − |
| 4 | scAAV4.CB6-eGFP | 6.5 × 10¹² GC/mL | − | + | − |
| 5 | scAAV5.CB6-eGFP | 1.4 × 10¹³ GC/mL | − | + | ++ |
| 6 | scAAV6.CB6-eGFP | 8 × 10¹² GC/mL | − | + | ++ |
| 7 | scAAV6.2.CB6-eGFP | 8 × 10¹² GC/mL | − | + | ++ |
| 8 | scAAV7.CB6-eGFP | 1.5 × 10¹³ GC/mL | − | + | ++ |
| 9 | scAAV8.CB6-eGFP | 7 × 10¹² GC/mL | − | − | ++ |
| 10 | scAAV9.CB6-eGFP | 1.5 × 10¹³ GC/mL | − | ++ | ++ |
| 11 | scAAVrh8.CB6-eGFP | 8 × 10¹² GC/mL | − | − | − |
| 12 | scAAV10.CB6-eGFP | 8 × 10¹² GC/mL | − | − | ++ |
| 13 | scAAVrh39.CB6-eGFP | 1 × 10¹³ GC/mL | − | − | ++ |
| 14 | scAAVrh43.CB6-eGFP | 6 × 10¹² GC/mL | − | − | ++ |
| 15 | scAAV2/2-66.CB6-eGFP | 1 × 10¹³ GC/mL | − | − | − |
| 16 | scAAV2/2-84.CB6-eGFP | 4 × 10¹² GC/mL | − | − | − |
| 17 | scAAV2/2-125.CB6-eGFP | 5 × 10¹² GC/mL | − | − | − |

Among 17 AAV serotypes, scAAV1, 5, 6, 6.2, 7, and 9 were effective to transduce both mouse primary OCs and Raw264.7 OC line while scAAV8 and 10, scAAVrh39 and 43 transduced only Raw264.7 OC line. (representative images in FIGS. 3 and 4).

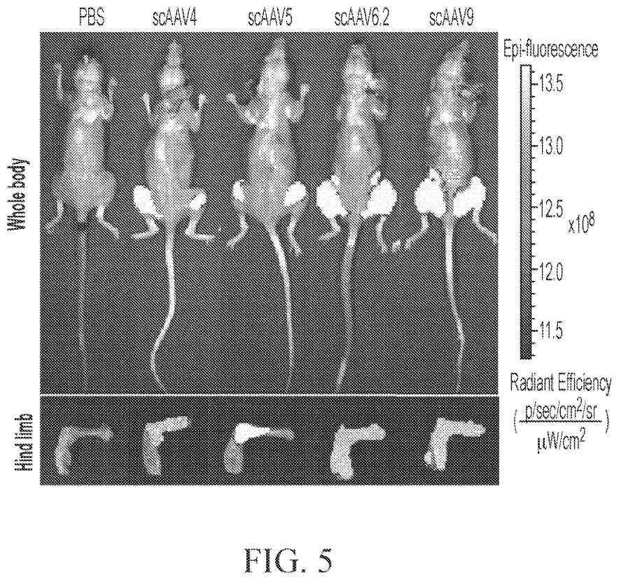

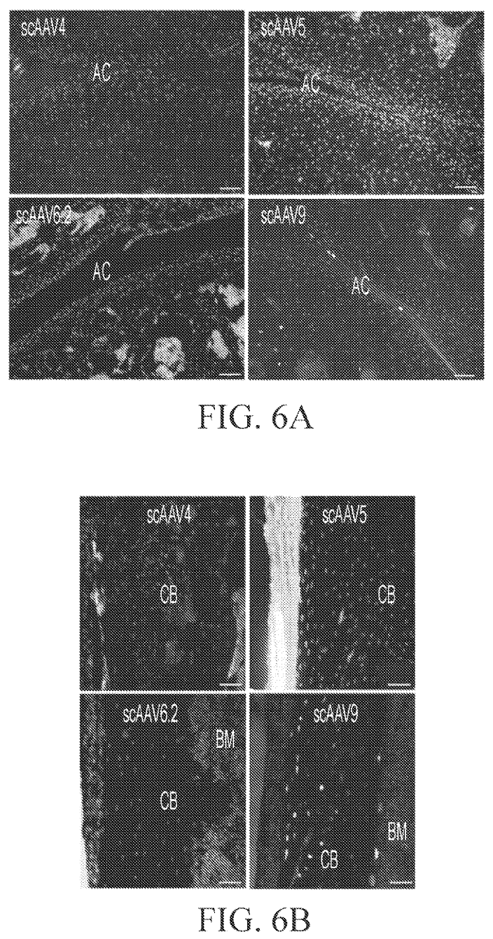

To examine the tropism of AAV capsids to articular cartilage and bone, eight scAAV-Egfp vectors selected from the in vitro screen (scAAV1, scAAV4, scAAV5, scAAV6, scAAV7, scAAV9, seAAVrh.10, and scAAVrh.39) . To test their ability to transduce chondrocytes on articular cartilage and/or OBs and/or OCs on bone surface, a single dose of 10¹²˜10¹³/ml genome copies of the eight selected GFP-encoding AAV serotypes was injected into both knee joints of 2 month old mice (C57BL/6J) and 4 weeks later, mice were euthanized, and AAV transduction to articular cartilage and/or bone was monitored by the IVIS-100 optical imaging system at the UMMS optical imaging facility (FIG. 5). As a negative control, mice were injected with PBS. Little to no expression of EGFP in the articular cartilage (FIG. 28) or the growth plate (FIG. 22) for the majority of capsids. This discrepancy may be due to the poor accessibility of scAAV vectors to chrondrocytes, where are embedded in the avascular microenvironment of these structures. Alternatively, vectors may simply exhibit a lower infectivity of primary chondrocytes in adult mice. GFP proteins were highly expressed in the knee joints and hindlimbs and their expression was restricted to the local injection area (FIG. 5, top, FIG. 28). In particular, GFP expression was detected in the bone and the knee joints even after removal of the muscles (FIG. 5, bottom). To confirm GFP expression in chondrocytes in the articular cartilage and/or in OBs and/or OCs on the bone surface, histology on frozen sections of the knee joints and femurs was performed. As seen in FIG. 6A, no or little expression of GFP proteins in articular cartilage was detected in the knee joints injected with scAAVI, scAAV4, scAAV5. scAAV6, seAAV7, scAAV9, seAAVrh.10, and scAAVrh.39. In the knee joints injected with scAAV9, GFP expression was detected in a small population of chondrocytes. In contrast to in vitro data using the chondrocyte precursor line ATDC5, these results indicate that certain scAAV serotypes are not effective to transduce chondrocytes in articular cartilage in vivo.

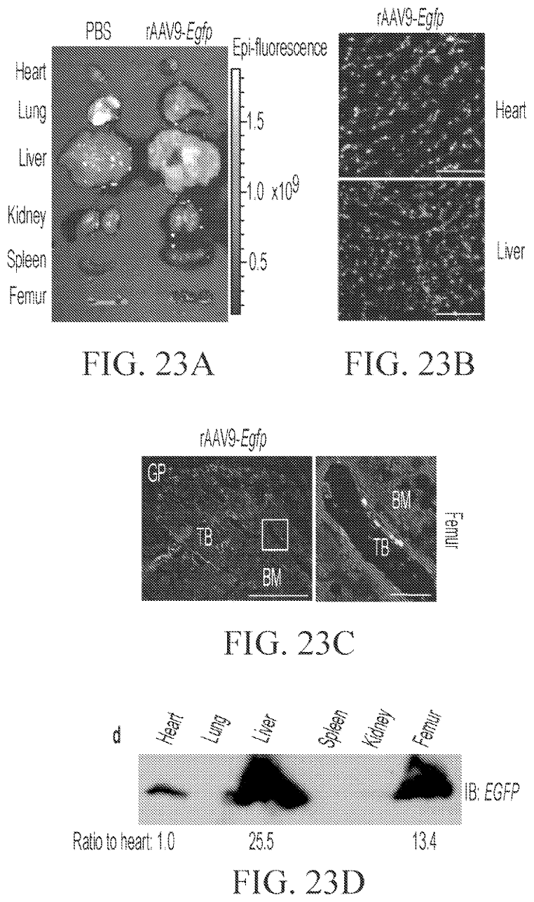

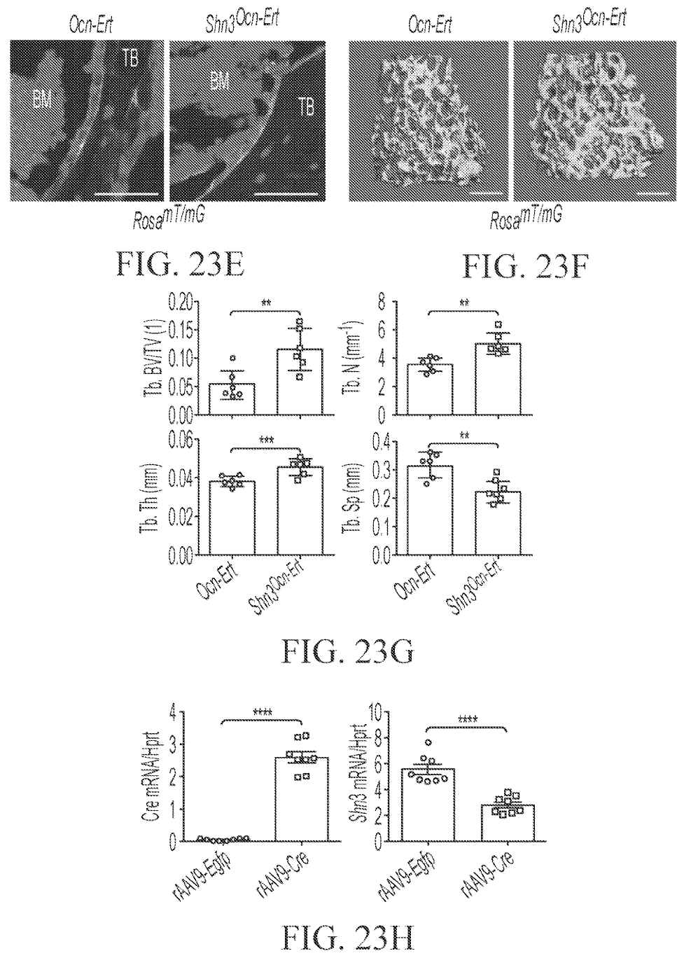

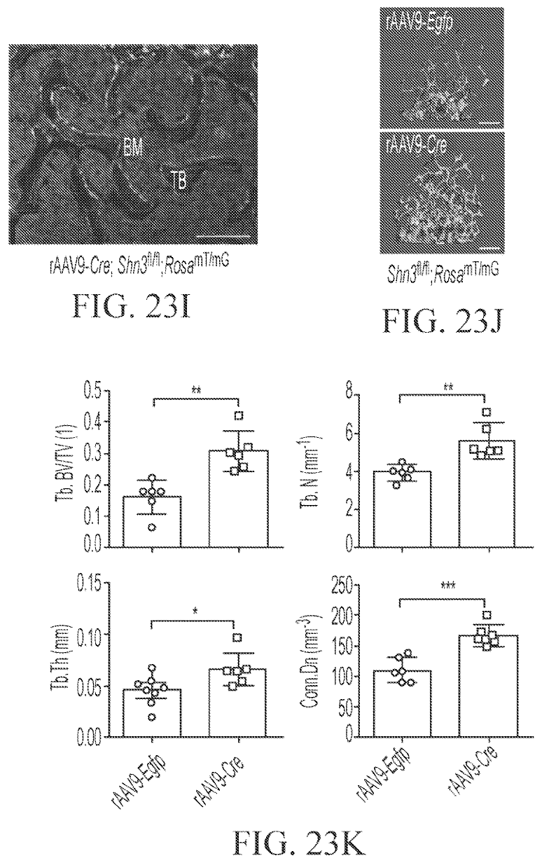

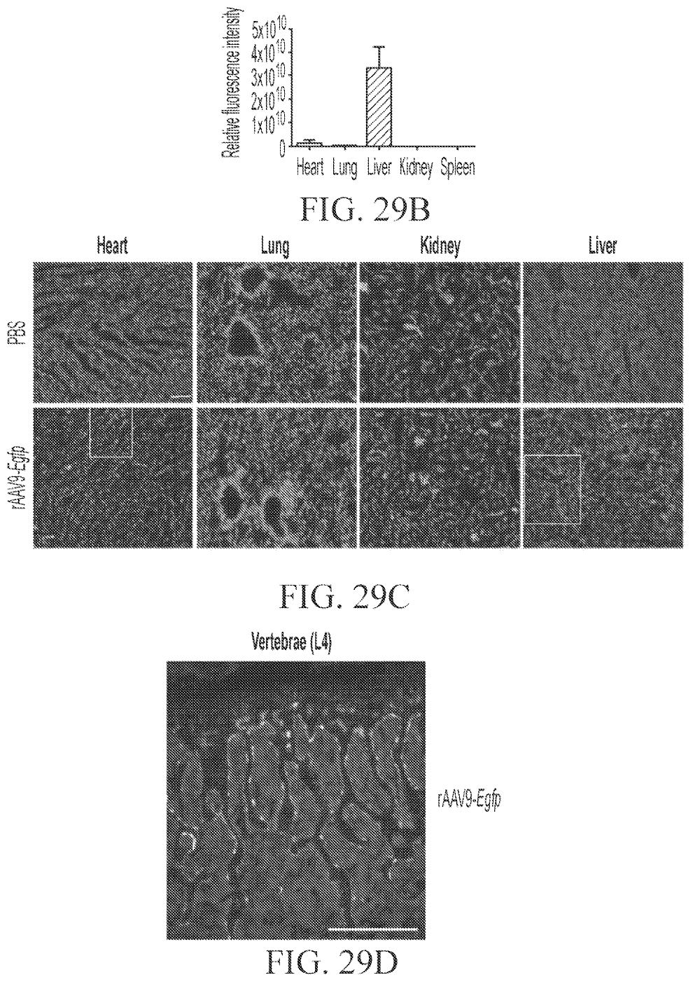

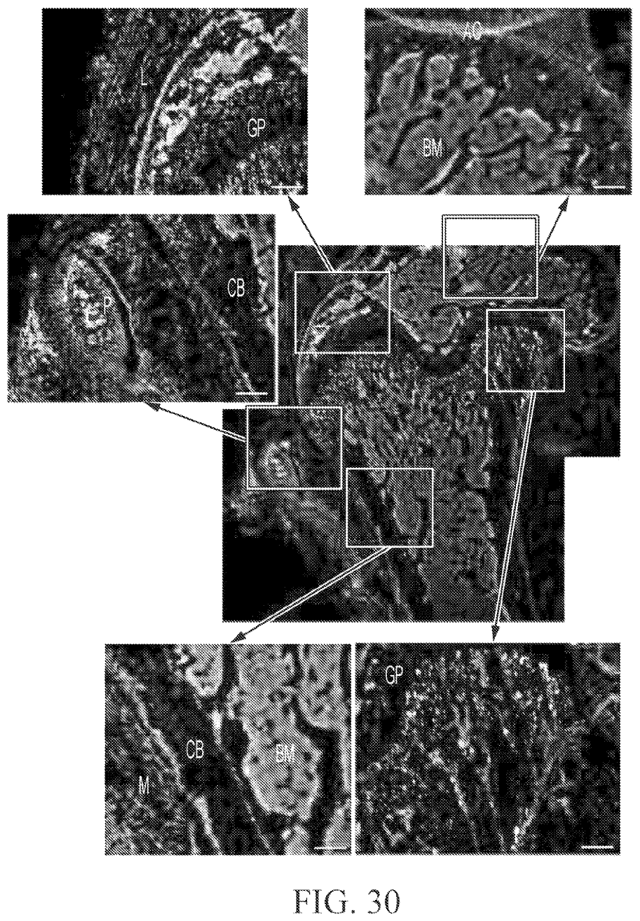

By contrast, GFP proteins were highly expressed in OBs and osteocytes that reside in cortical bones when scAAV9 was injected into the knee joints. Whole body and individual organ imaging of treated mice showed that EGFP expression was highest in the liver and hindlimbs (FIGS. 23 and 29). Expression in the heart and femur was modest, while expression in the lung, kidney, and spleen was not detected. Expression of EGFP in the heart, liver, and femur was further confirmed by fluorescence microscopy and by immunoblot analysis (FIG. 23). As observed, EGFP protein was primarily expressed in endosteal osteoblasts and osteocytes in cortical and trabecular bones, but not in the ligament, articular cartilage, growth plate, periosteal osteoblasts, bone marrow, and patellta (FIGS. 23 and 30). These results demonstrate that systemically delivered scAAV9 vector targets osteoblast lineage cells residing in the endosteal bone.

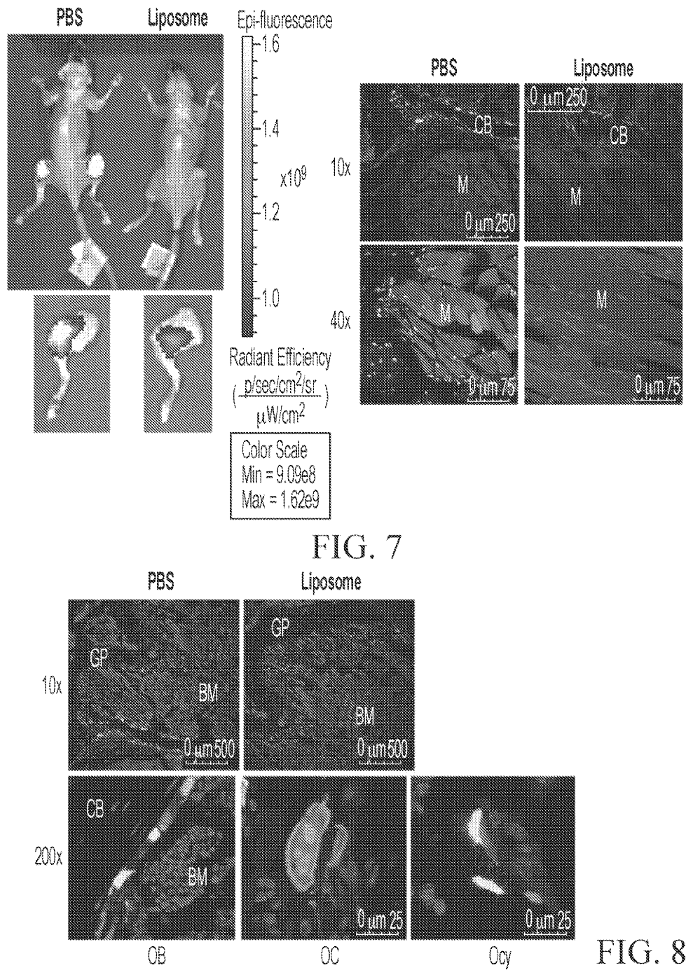

In addition to scAAV9, a high GFP expression in periosteal cells and a low GFP expression in osteocytes in cortical bones was observed in the treatment of scAAV5. A small population of periosteal cells expressed GFP proteins when injected with scAAV4 whereas no GFP expression was detected in the treatment of scAAV6.2 (FIG. 6B). Thus, these results indicate that scAAV9 serotype is most effective to transduce OBs and OCs on the bone surface in mice. To increase transduction efficacy of scAAV9 to bone tissue, scAAV9 was formulated with PBS or X-tremeGene 9 DNA transfection reagent (Roche), a blend of lipids with extremely low cytotoxicity, and injected into knee joints of mice (FIGS. 7 and 8). When formulated with X-tremeGene 9 (liposome), GFP proteins were highly expressed in active OBs and OCs under growth plate and on trabecular bone surface (epiphysis) as well as terminally differentiated OBs, osteocytes in cortical bones (diaphysis). However, GFP expression was markedly reduced in the muscle, indicating that X-tremeGene 9 formulation alters the in vivo tropism of scAAV9.

### Development of Bone Targeting-seAAV9 (BT-scAA9) Serotype via Genetic or Chemical Modifications of Capsid Proteins

To maximize AAV-mediated gene therapy in osteoporosis, it is desirable for scAAV9serotype to home to and accumulate on the bone surface where OBs and OCs reside. It has been observed that bone-targeting peptides, ((AspSerSer)₆, DSS) are effective in directing osteogenic siRNA-encapsulated liposomes to the bone-formation surface. Additional bone-targeting peptide, HABP-19 (CγEPRRγEVAYELγEPRRγEVAγEL; SEQ ID NO: 17) has been reported to selectively bind to the bone hydroxyapatite in the culture as well as in mice. HABP-19 is a biomimic of osteocalcin, the most abundant non-collagenous protein secreted from OBs. γ(Gla residue)-carboxylated glutamic acid (Glu) is derived from Glu by vitamin K-dependent γ-carboxylation.

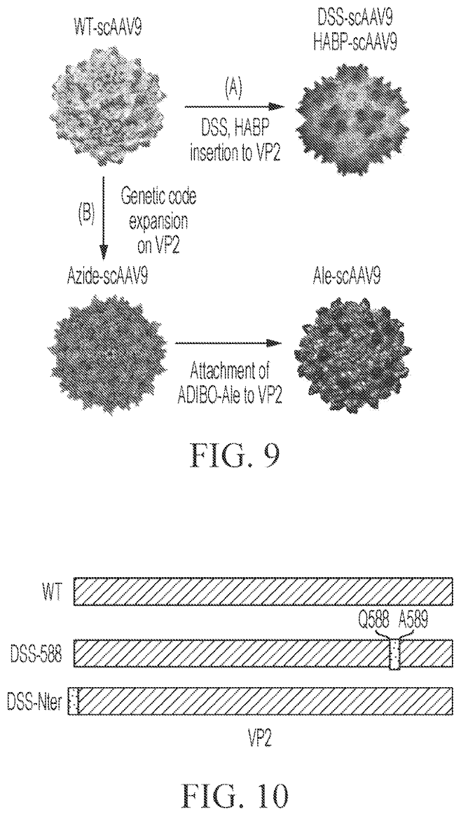

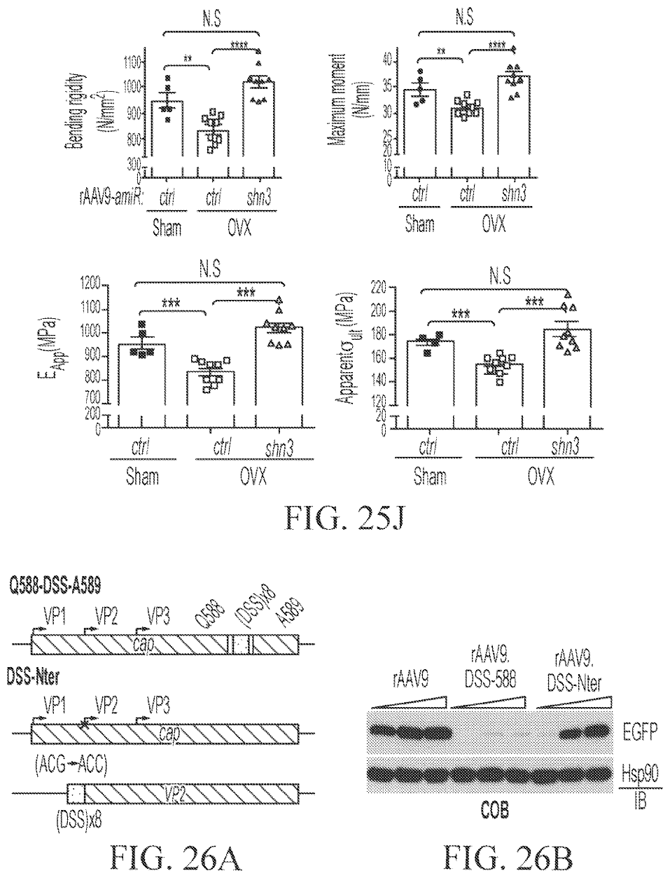

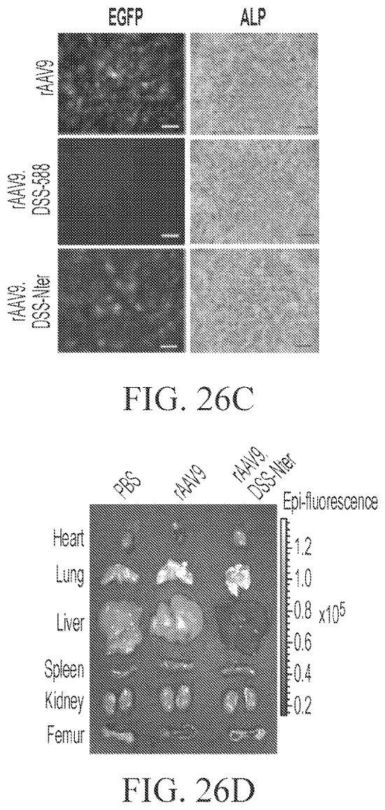

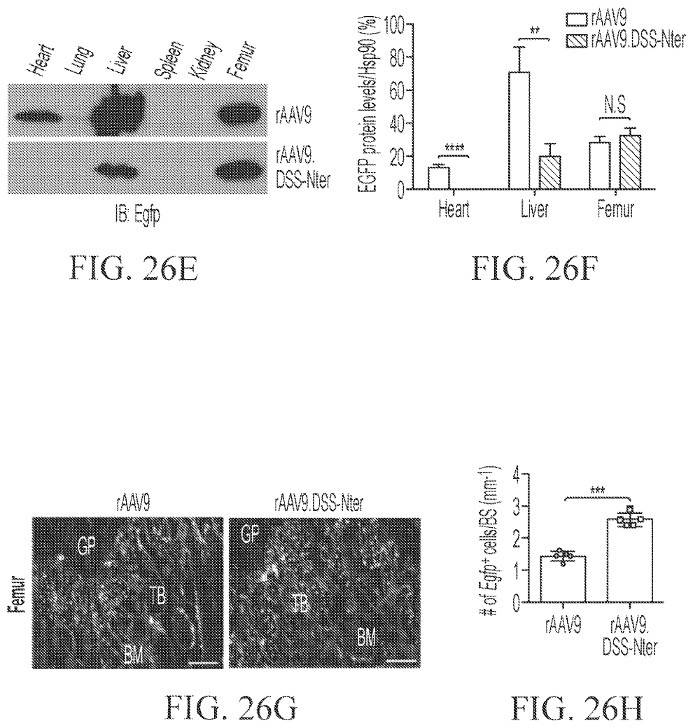

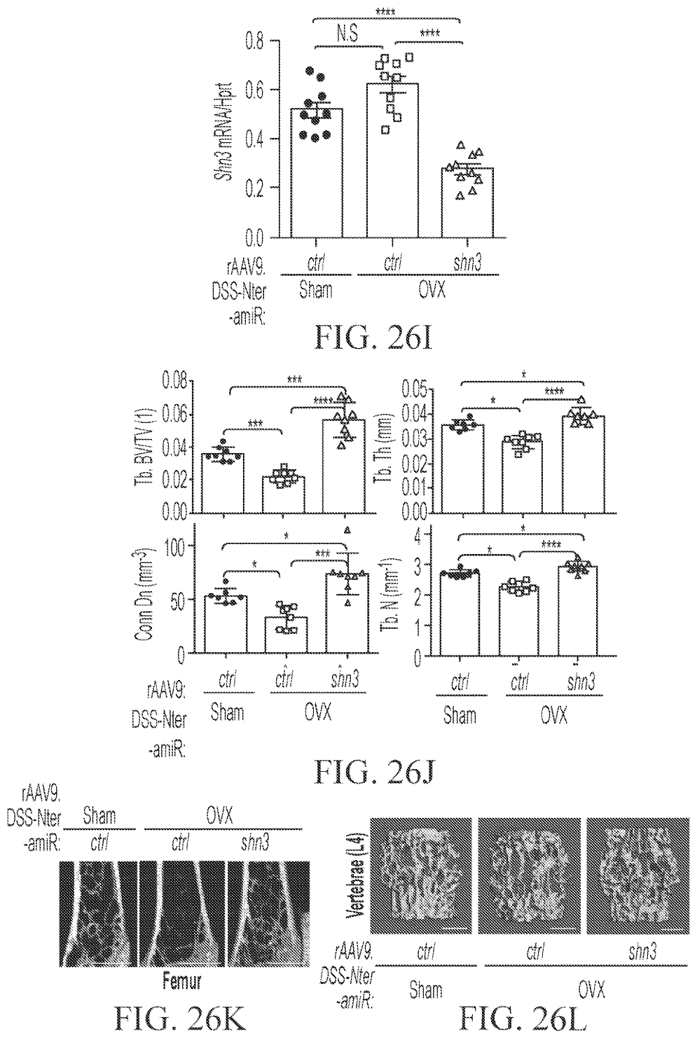

This example describes inserting bone-targeting peptides into AAV capsids in order to direct engineered scAAV9 serotype to the bone (FIG. 9A, DSS-scAAV9, HABP-scAAV9). The DNA sequences encoding (AspSerSer)₆ (SEQ ID NO: 16) are inserted into the capsid protein VP2 open reading frame (ORF) between glutamine 587 and alanine 588 (DSS-587), between glutamine 588 and arginine 589 (DSS-588) or N-terminus of VP2-ORF (DSS-Nter) using a standard cloning method (FIGS. 10 and 26). Additionally, AAV capsids conjugated to a validated bone-seeking molecule via a chemical reaction (FIG. 9B) are produced. It has been observed that attachment of a bone-seeking molecule (ADIBO-Ale) to azide moieties on the cell surface significantly enhanced the ability of non-bone cells (e.g., Jurkat T leukemia line) to bind to bone fragments.

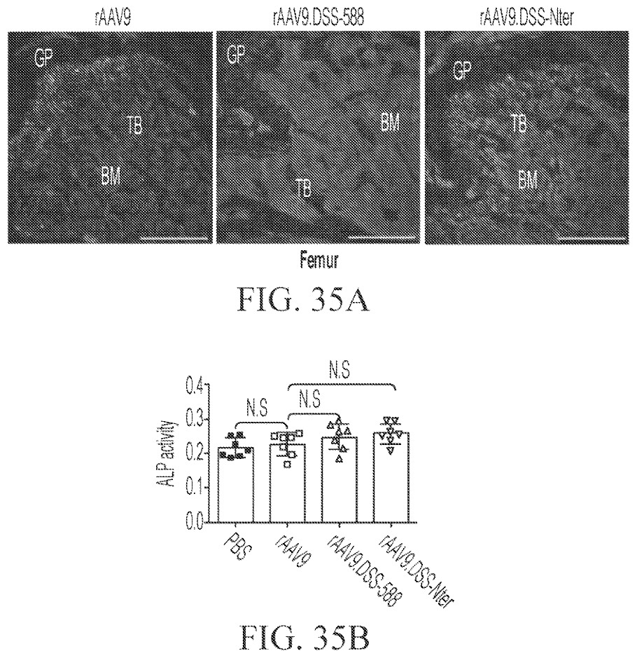

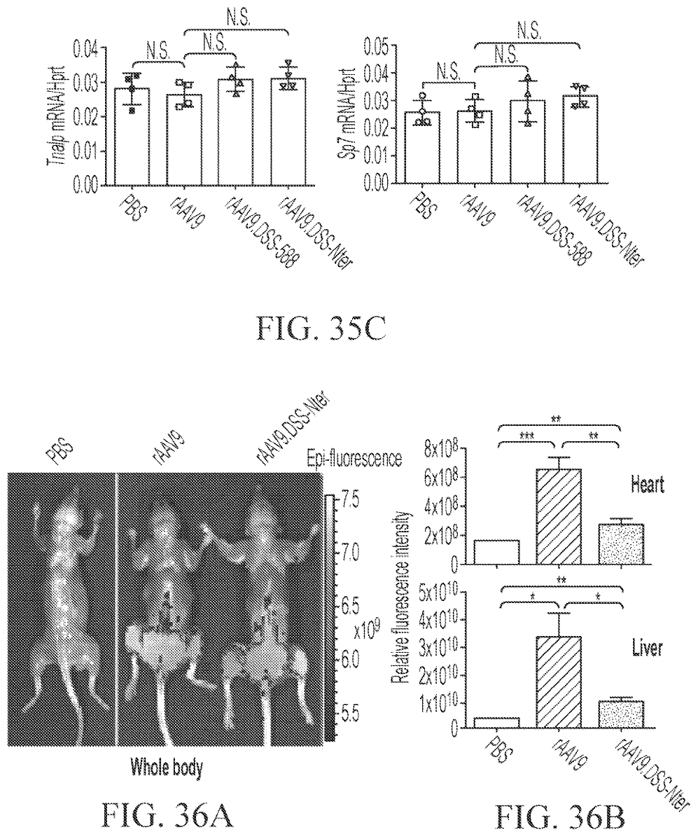

To examine whether the DSS insertion affects infectivity of scAAV9, vectors packaged into prototypical AAV9, AAV9. DSS-587, or AAV9. DSS-Nter capsids were infected into COBs, and EGFP expression was assessed by immunoblotting or by fluorescence microscopy (FIG. 29). Compared to seAAV9, the seAAV9. -DSS-Nter showed a modest reduction in infectivity at lower MOI (10⁹ GC/mL), whereas little EGFP expression was detected in COBs treated with scAAV9. DSS-587 (FIG. 29). Similarly, when treating mice via i.a. injections, scAAV9, and scAAV9. DSS-Nter strongly transduced femurs as before (FIG. 35). In contrast, the scAAV9. DSS-587 yielded little to no transgene expression. Additionally, alkaline phosphatase (ALP) activity and expression of osteogenic genes were comparable in COBs treated with scAAV9 and scAAV9. DSS vectors, indicating no adverse effects by seAAVs on osteoblast differentiation (FIGS. 29 and 35).

Engineered scAAV9 serotype (Ale-scAAV9), which is decorated with the bone-targeting molecule on the capsid proteins, is tested. In order to create azide-bearing amino acids on AAV capsid proteins, asparagine 587 in the VP3 capsid protein is changed to the amber codon (TAG) or this amber codon is inserted between asparagine 587 and arginine 588. Together with these mutant VP3 plasmids, pHelper, pAAV-GFP, and pPyIRS/IRNACUA are transiently transfected into AAV-293 packaging cells at a molar ratio of 1:1:1:2 using a DNA transfection reagent. Six hours later, the medium is replaced with fresh medium containing 1 mM N-2-azideoethyloxycarbonyl-L-lysine (NAEK) and 72 hours later, scAAV9 serotype with azide-bearing amino acids (Azide-scAAV9) is harvested from the transfected cells. The orthogonal amber suppressor aminoacyl-tRNA synthetase/IRNA-CUA pairs expressed from the pPyIRSARNACUA plasmid synthesize an azide-bearing unnatural amino acid at the site of amber codon in the presence of NAEK.

Expression of the azide moiety-containing VP3 mutants is confirmed by immunoblotting with anti-VP3 antibody. Once confirmed, the ability of Azide-scAAV9 serotype to transduce OBs or OCs is assessed by monitoring GFP expression. Primary mouse OBs are treated with GFP-encoding Azide-scAAV9 serotype and GFP expression is assessed by epifluorescence microscopy. Additionally, these scAAV9 serotypes are used for infection of mouse BM-MO (OC precursors) in the presence of M-CSF and Rank ligand. Their transduction efficiency is compared with GFP-encoding WT-scAAV9 serotype.

Next, bone-targeting scAAV9 serotype (Ale-scAAV9) is produced by cross-linking the bone-seeking molecule (ADIBO-Ale) to azide-bearing amino acids in the VP3 capsid proteins of the Azide-scAAV9 serotype via a click chemistry. Azide-scAAV9 serotype is incubated with different concentrations of ADIBO-Ale (FutureChem) at room temperature and 2 hours later, unbound ADIBO-Ale is removed using dialysis. The ability of Ale-seAAV9 serotype to transduce primary OBs or OCs is examined by monitoring GFP expression.

### Validation of DSS-scAAV9 Transduction to Bone Cells and Tissue

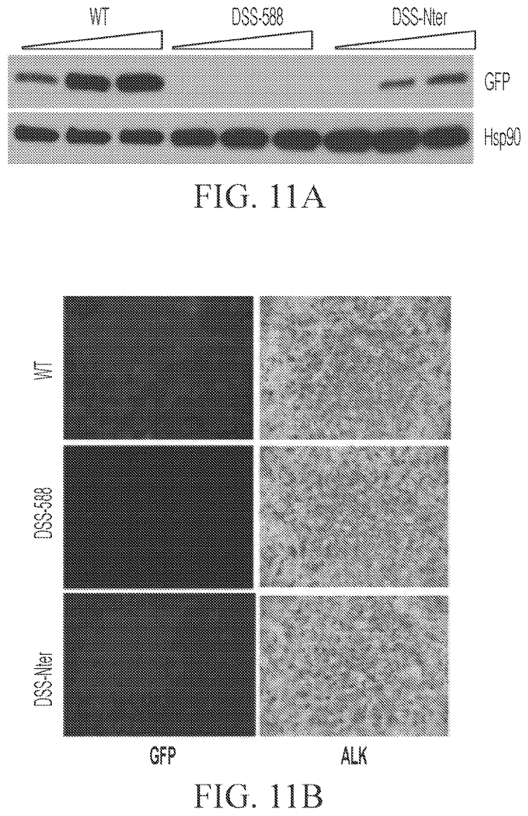

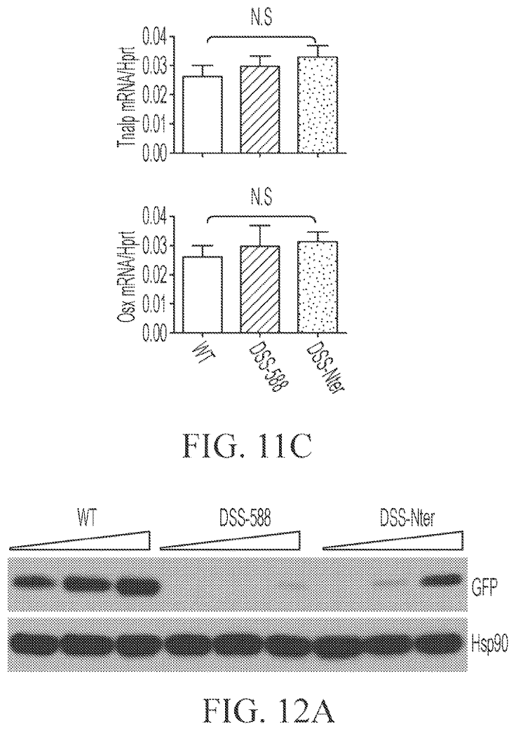

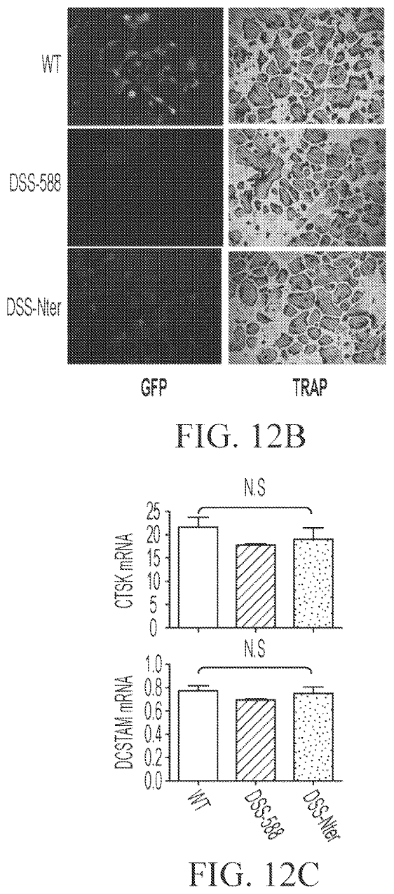

It has been previously reported that when peptides are inefficiently or inappropriately fused into AAV capsids, the transduction efficiency of AAV can be markedly reduced. To test effects of the DSS peptide insertion on the ability of scAAV9 to transduce to OBs and OCs, mouse OB and OC precursors were treated with different doses of GFP-encoding scAAV98 (WT-, DSS-588, DSS-Nter). 2 days later, GFP expression was analyzed by western blotting using anti-GFP antibody and by epifluorescence microscopy. As seen in FIGS. 11 and 12 (A and B-left), little or no expression of GFP proteins was detected in both OBs and OCs when treated with the DSS-588. By contrast, compared with WT-scAAV9, treatment of DSS-Nter scAAV9 showed a decrease in GFP expression in OBs and OCs and, its expression was increased in a dose-dependent manner. Differentiation of OBs (FIG. 11B—right and 11C) and OCs (FIG. 12B—right and 12C) is normal in the treatment of these seAAV9, demonstrating that scAAV9transduction does not affect OB and OC function.

Next, the ability of DSS-scAAV9 vectors to transduce to bone cells in vivo was investigated. GFP-encoding scAAV9 vectors (WT-, DSS-588, DSS-Nter) were formulated with X-tremeGene 9 and a single dose of 10¹²˜10¹³/ml genome copies these scAAV9 vectors was injected into knee joints of 2 months old mice (C57BL/6J) 1 week later, histology was performed on frozen sections of the femur and GFP expression in OBs, OCs, and/or osteocytes was assessed using a confocal microscopy (FIG. 13). PBS injection serves as a non-GFP expressing control. Similar to in vitro experiments (FIGS. 11 and 12), little or no GFP expression was observed in the femur treated with DSS-588 scAAV9. Although a population of GFP-expressing cells were reduced compared to WT-scAAV9, DSS-Nter scAAV successfully transduced all of the bone cells, including OBs, OCs, and osteocytes.

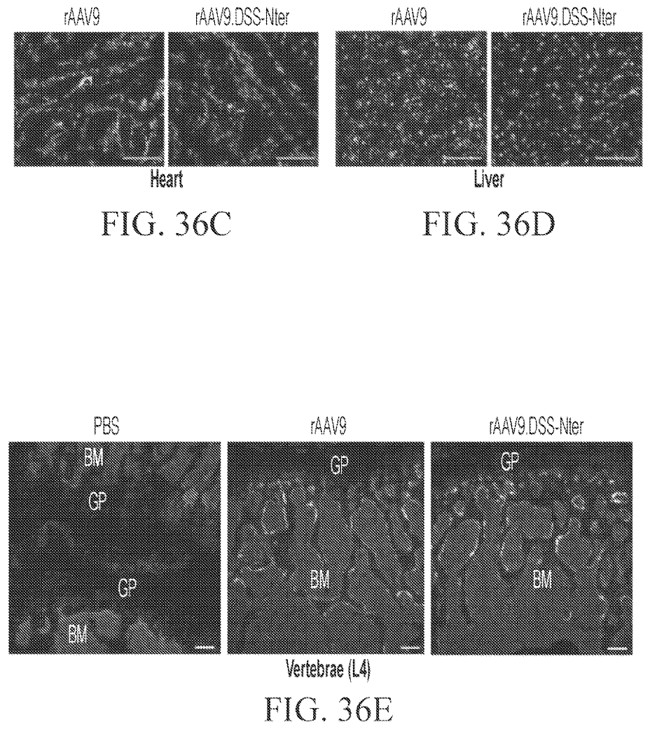

The AAV capsid was tested for bone-targeting activity in vivo. As before, scAAV9 or scAAV9-DSS-Nter was i.v. injected into two-month-old mice and tissue distributions were assessed by EGFP expression two weeks post-injection. While equivalent levels of EGFP protein were observed in the whole bodies of mice systemically treated with either scAAV9 or scAAV9-DSS-Nter (FIG. 36), mice injected with the seAAV9. DSS-Nter yielded EGFP expression levels of˜55% and˜75% of those achieved by scAAV9 in the heart and liver, respectively (FIG. 26 and FIG. 36). Importantly, expression in femurs and lumbar vertebrae were relatively comparable between treatment groups (FIGS. 26 and 36). Notably, scAAV9. DSS-Nter-treated femurs showed increased numbers of EGFP-expressing cells in comparison to seAAV9-treated femurs (FIG. 26). These results demonstrated that the engineered VP2 capsid protein fused with the bone-targeting peptide motif (DSS) improves bone-homing specificity of scAAV9 by detargeting delivery to non-skeletal tissues, and in turn, increase its tropism to the bone.

### Validation of Systemic Delivery of BT-scAAV9 Serotypes for Specific Bone Transduction in Vivo

To examine the ability of BT-scAAV9 serotypes to home to the bone in vivo, a single dose of 1×10¹⁰ genome copies of GFP-encoding scAAV9 vectors (WT-, DSS-or HABP, Ale) is administered locally or systemically into mice. For local administration, these scAAV9 serotypes are injected into the knee joints of 2 months old mice (C57BL/6J) and 1 week or 4 weeks later, AAV delivery to OBs, OCs, and/or osteocytes on the bone is monitored by an IVIS-100 optical imaging system. Additionally, histology is performed on frozen sections of the femur to detect GFP-expressing OBs. OCs, and/or osteocytes transduced by these scAAV9 serotypes. PBS injection serves as a non-GFP expressing control. For systemic administration, GFP-encoding WT-, DSS-, HABP-or Ale-scAAV9 serotype are administered to mice via intravenous (IV) or intraperitoneal (IP) injection. 1 week or 4 weeks later, AAV delivery to the bone is monitored by the IVIS-100 optical imaging system and GFP-positive bones will be further frozen-sectioned for histology.

### Generation of scAAV9 Vectors Encoding shRNAs Specific to Mouse Schnurri-3 (SHN3), Sclerostin (SOST), or Cathepsin K (CTSK)

Sclerosteosis is a rare genetic disorder with high bone mass, and has been observed to be associated with a loss-of-function mutation in SOST gene, the gene that encodes sclerostin. Sclerostin is secreted from osteocytes, terminally-differentiated osteoblasts, and inhibits bone formation and enhances bone resorption. Treatment with a monoclonal human anti-sclerostin antibody restores bone loss in human patients and animal models with osteoporosis by promoting bone formation and inhibiting bone resorption. However, FDA approval is currently unlikely due to an increased risk of stroke. Unlike sclerostin that inhibit osteoblast differentiation, Cathepsin K (CTSK), a lysosomal cysteine proteinase, is highly expressed in mature osteoclasts important for bone resorption. Pycnodysostosis, a rare genetic disorder with high bone mass, results from inactivating mutations in CTSK gene in humans. Treatment with a small molecule inhibitor of CTSK prevents bone loss in human patients and animal models with osteoporosis by suppressing bone resorption while limiting adverse effects on bone regeneration activity. However, it was recently withdrawn from FDA consideration due to an elevated incidence of stroke.

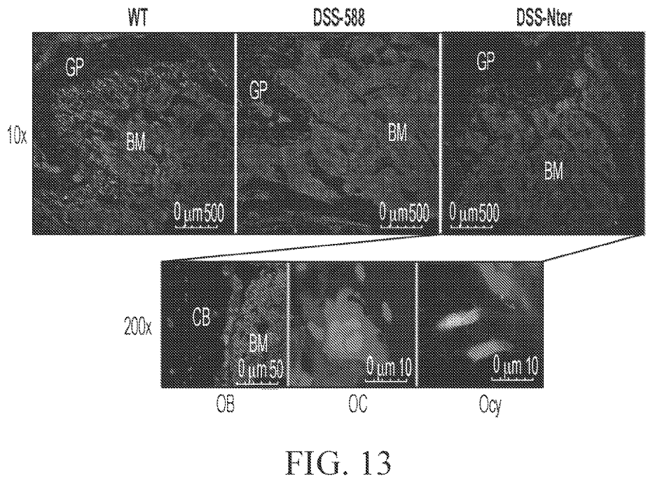

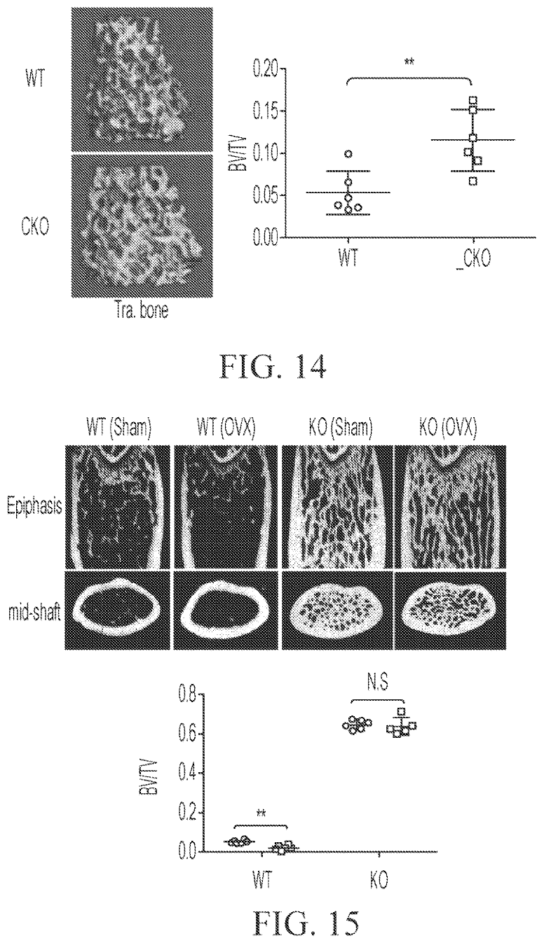

Schnurri-3 (SHN3) as a novel suppressor of osteoblast differentiation. Transient deletion of SHN3 in OBs using an inducible loxP: Cre system resulted in a significant increase in bone mass in mice, indicating that down-regulation of SHN3 expression provides an attractive therapeutic approach to restore low bone mass in osteoporosis (FIG. 14). Likewise, inhibition of SHN3 prevents bone loss in a mouse model of osteoporosis by promoting bone formation (FIG. 15). However, unlike sclerostin and CTSK, SHN3 deficiency is specific to bone and is not associated with phenotypes in non-skeletal tissues. Thus, the bone-targeting seAAV9 vectors described in Example 1 were used subsequently for vectors to silence mouse SHN3, SOST, or CTSK in OBs and/or OCs in vitro and in vivo.

Two shRNA hairpins targeting the above-noted genes (e.g., SHN3, CTSK) were adapted to surrogate mutant inverted terminal repeats (mTRs) that is required for functional self-complementary AAV vectors (e.g., mTRs) and the modified shRNA-mTR sequences are described in the Sequences section. These modified shRNA-mTRs were cloned into the pAAVscCB6-EGFP vector at the restriction enzyme sites, PstI and Bg12, and packaged to scAAV9 capsids.

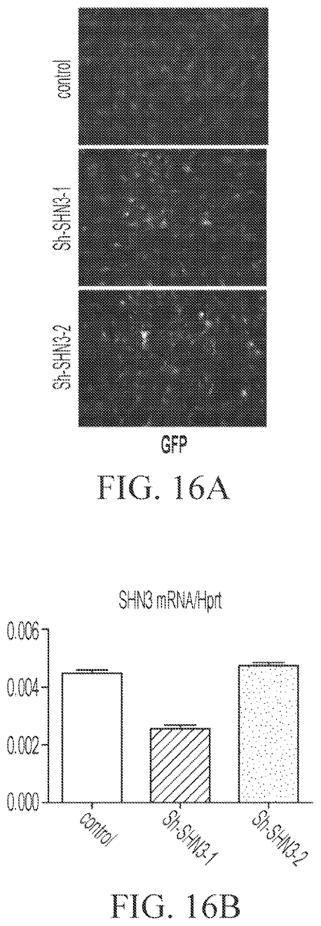

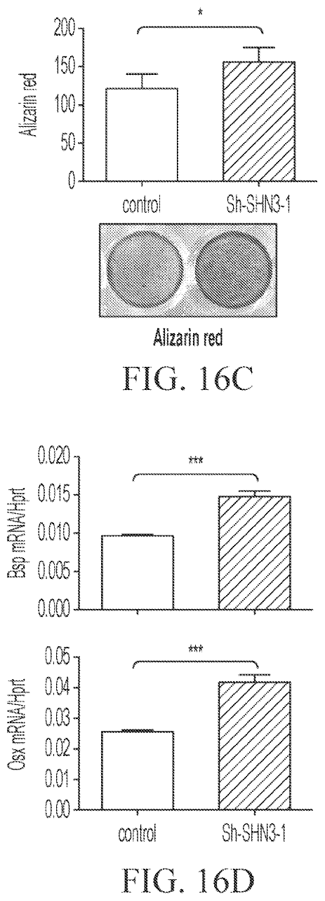

The purified scAAV9 vectors expressing two shRNA hairpins specific to mouse schnurri-3 (SHN3) and mouse cathepsin K (CTSK) (scAAV9-mSHN3i and scAAV9-mCTSKi) were produced. For in vitro characterization, infectivity, knockdown efficiency, and functional activity of scAAV9-mSHN3i and scAAV9-mCTSKi were tested in OBs and OCs, respectively. First, a single dose of 10¹²˜10¹³/ml genome copies of two scAAV9-mSHIN3is (Sh-SHN3-1, -2) or scAAV-vector control (control) were treated with mouse primary OB precursors. 2 days after transduction, GFP expression was monitored by epifluorescence microscopy and knockdown efficiency were assessed by measuring SHIN3 mRNA levels using RT-PCR. OB differentiation was assessed by measuring expression of OB differentiation genes (e.g., bone sialoprotein (BSP) and osterix (OSX)) and mineralization activity by alizarin red staining at day 6 and 15 of the OB culture, respectively (FIGS. 16A-16D). Transduction of seAAV9-mSHN3is to OBs was as effective as that of scAAV9-control. The scAAV9-mSHN3i (Sh-SHN3-1) was able to decrease SHN3 mRNA levels by 50% (FIGS. 16A-16B). SHN3 knockdown increased mineralization activity and expression of OB differentiation genes (FIGS. 16C-16D).

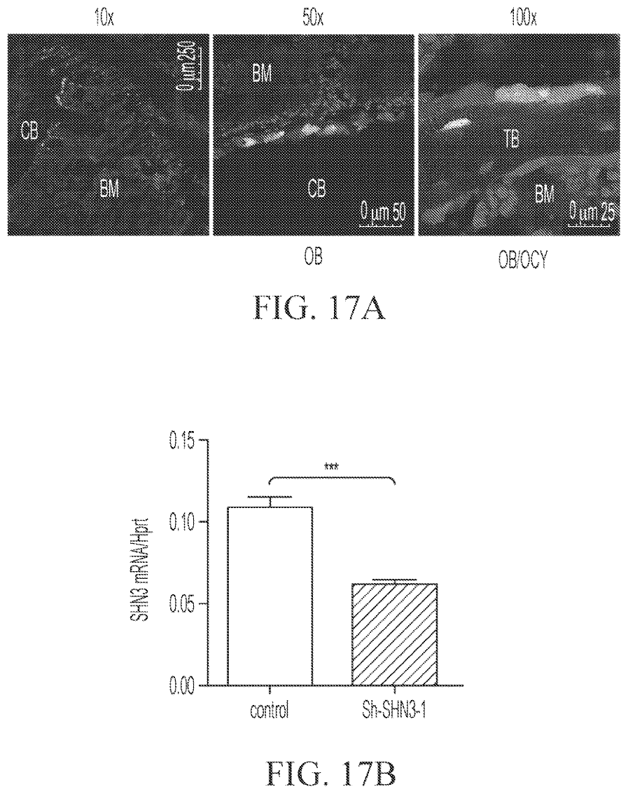

Next, transduction and knockdown efficacy of scAAV9-mSHN3i vector in vivo was examined (FIG. 17). GFP-encoding scAAV9 vector (Sh-SHN3-1) was formulated with X-tremeGene 9 and a single dose of 10¹²˜10¹³/ml genome copies of this scAAV9 vector was injected into knee joints of 2 months old mice (C57BL/6J). 1 week later, histology was performed on frozen sections of the femur and GFP expression in OBs and osteocytes was assessed using a confocal microscopy. PBS injection serves as a non-GFP expressing control. Similar to control scAAV9, GFP expression in OBs and osteocytes was observed in the femur treated with scAAV9-mSHN3i vector (FIG. 17A). Alternatively, GFP-expressing cells were isolated from the femur using FACS sorting and SHN3 mRNA levels were assessed by RT-PCR, demonstrating 50% reduction in SHIN3 mRNA levels by transduction of scAAV9-Sh-SHN3-1 vector (FIG. 17B).

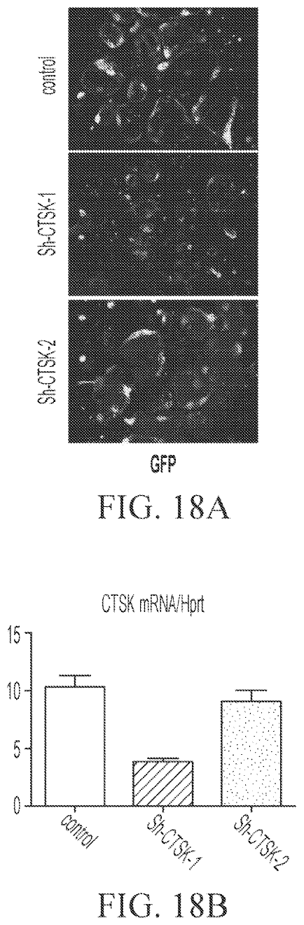

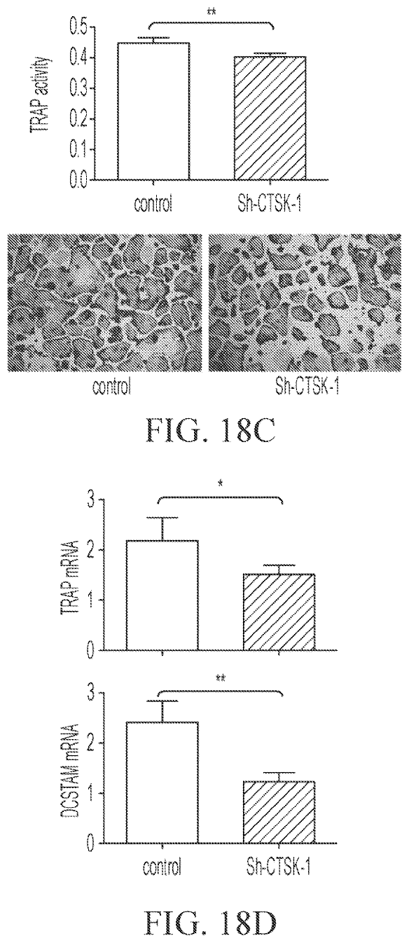

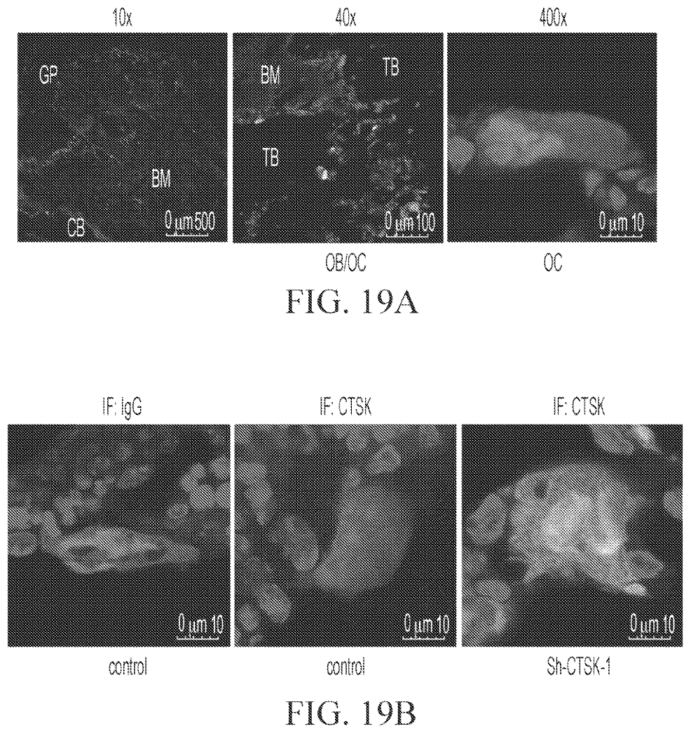

Additionally, infectivity, knockdown efficiency, and functional activity of scAAV9-mCTSKi was also investigated in mouse OCs. To obtain OC precursors, mouse monocyte line, Raw264.7 were treated with Rank ligand (5 ng/ml) for 2 days. Subsequently, these OC precursors were treated with a single dose of 10¹²˜10¹³/ml genome copies of two scAAV9-mCTSKis (Sh-CTSK-1, -2) or scAAV-vector control (control) for 2 days. Transduction efficiency was assessed by GFP expression using epifluorescence microscopy (FIGS. 18A-18D) and knockdown efficiency were assessed by measuring CTSK mRNA levels using RT-PCR (FIG. 18B). Additionally, OC differentiation was assessed by measuring TRAP enzymatic activity, muti-nucleation of TRAP-stained OCs, and expression of OC differentiation genes (e.g., dendritic cell-specific transmembrane protein (DC-STAMP) and TRAP) (FIGS. 18C and 18D). Transduction of scAAV9-mCTSKis to OC precursors was as effective as that of scAAV9-control. The scAAV9-mCTSKi (Sh-CTSK-1) was able to decrease CTSK mRNA levels by 70% (FIGS. 18A-18B). CTSK knockdown resulted in a mild decrease in OC differentiation (FIGS. 18C-18D). To examine the ability of scAAV9-mCTSKi vector to transduce to OCs in vivo, a single dose of 10¹²˜10¹³/ml genome copies of formulation of GFP-encoding scAAV9 vector (Sh-CTSK-1) with X-tremeGene 9 was injected into knee joints of 2 months old mice (C57BL/6J). Immunofluorescence analysis to GFP and CTSK expression confirmed that both of control-scAAV9 and, scAAV9-mCTSKi vectors can transduce OCs on the bone surface in vivo (FIG. 19A). CTSK expression was markedly reduced in GFP-expressing OCs only when transduced with scAAV9-mCTSKi vector (FIG. 19B).

Knockdown efficiency of the pAAVscCB6 vector encoding two shRNA hairpins specific to mouse sclerostin (SOST) shRNAs (scAAV9-mSOSTi) was validated in GFP-DMP1-expressing osteocyte line with high expression of sclerostin. Once generated, a single dose of 10¹²˜10¹³/ml genome copies of two scAAV9-mCTSKi or scAAV9-vector control (control) is injected into a mouse osteocyte line with high expression of sclerostin. 2 days after transduction, GFP expression is assessed by Western blotting, and the knockdown efficiency is assessed by measuring SOST mRNA levels using RT-PCR.

### Development of Gene Therapeutics for Osteoporosis using BT-AAV-Mediated Gene Silencing

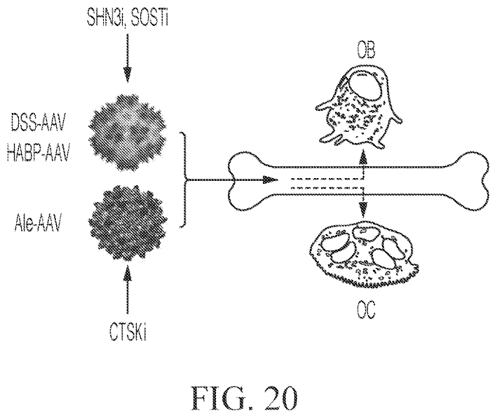

This example describes testing of functional efficacy of the scAAV9 serotypes encoding shRNA hairpins specific to mouse SHN3. SOST, CTSK (SHN3i, SOSTi, CTSKi), shRNA hairpins are cloned into GFP-encoding AAV vectors and packaged into the BT-AAV capsids (DSS-scAAV9, Ale-scAAV9) (FIG. 20). As a negative control, control ShRNA (cont-i) is cloned into the AAV vector. BT-AAV serotypes encoding cont-i, SHN3i, or SOSTi are transduced into primary mouse OBs and 3 days later, knockdown efficiency of SOST or SHN3is assessed by quantitative PCR. Mouse primary OC precursors are transduced by BT-AAV serotypes encoding cont-i or CTSKi and cultured in the presence of M-CSF and RANK ligand. Three days after the transduction, knockdown efficiency of CTSK is analyzed by quantitative PCR, and 6 days later the ability of CSTK silencing to inhibit OC differentiation is assessed.

To examine the ability of these gene therapeutics to prevent bone loss in mouse models of osteoporosis, 4 weeks after sham or OVX surgery, 4-months-old female mice (C57BL/6), n=12 mice/group) are IV or IP injected with a single dose of 1×10¹² genome copies of these BT-scAAV9 serotypes. Alternatively, 22-months-old female mice (C57BL/6J, n=12 mice/group) are IV or IP injected. As a negative control, GFP-encoding scAAV9 vector with control shRNA (cont-i) is administered into these mice. Two months after treatment, mice are labeled with calcein and alizarin red and subjected to dynamic histomorphometry in order to assess the number of OBs and OCs, bone formation rate, and OC resorption activity in vivo. In order to locate GFP-expressing scAAV9-transduced cells, IVIS-100 optical imaging is performed in a whole body. GFP-expressing tissues are further processed for histology on frozen sections. Furthermore, scAAV9-transduced OBs and/or OC's that express GPP proteins are isolated from long bones using FACS sorting, and SOST, SHN3, or CTSK mRNA levels are assessed by quantitative PCR. For skeletal analysis, microCT of long bones and vertebrae is performed to analyze bone mass and structure.

Additionally, histologic sections are stained with tartrate-resistant acid phosphatase (TRAP) as a marker of OC differentiation and immunohistochemistry (IHC) for type I Collagen al (Coll) and Runx2 as markers of OB differentiation. This analysis is accompanied by quantitative PCR analysis of bone RNA measuring expression of OB differentiation genes. Lastly, as in human patients with osteoporosis, systemic OB and OC activity is analyzed by measuring the levels of serum bone turnover markers, including type I collagen C-terminal telopeptide (CTX), N-terminal propeptide of type 1 procollagen (PINP), and bone-specific alkaline phosphatase (BSAP) using ELISAs.

### Example 2: Development of Novel Gene Therapeutics for Osteoporosis Using AAV-Mediated Gene Addition

Intermittent treatment of recombinant PTH peptides (1-34 aa) or recombinant PTHrP peptides (1-36 aa) via subcutaneous injection, in some embodiments, increases OB activity and promotes bone formation in human patients and animal models with osteoporosis. These peptides (teriparatide, abaloparatide) are PDA-approved and currently used for human patients with osteoporosis. In some embodiments, secretary factor DJ-I functions as a mediator of the cross-talk between OBs and endothelial cells. Treatment with DJ-1, in some embodiments, promotes OB differentiation in human MSCs and angiogenesis in human endothelial cells while it suppresses OC differentiation.

This example describes delivery of scAAV9 serotypes encoding secretory osteogenic factors to animals via intramuscular (IM) injection through which the injected muscle serves as a bio-pump. providing stable, high expression of these factors in blood circulation to promote bone formation in the setting of osteoporosis (FIG. 21). To this end, cDNAs encoding human PTH (1-84 aa), human PTHrP (1-140 aa), or mouse DJ-1 (1-190 aa) is cloned into the pAAVscCB6-EGFP vector that contains a conventional signal peptide for protein secretion at the restriction enzyme sites. Agel and Hind3 vector, and packaged into muscle tropic AAV capsids (PTH-scAAV9, PTHrP-scAAV9, DJ-1-scAAV9). These cDNA sequences are described in the Sequences.

To validate the production of these secretory osteogenic factors in the AAV-transduced cells in vitro, scAAV9 serotypes are used to transduce mouse myoblast cell line (C2C12) and three days after culture, the supernatant is harvested and levels of secretory factors are measured by ELISAs. Alternatively, cells are lysed and intracellular proteins are assessed by immunoblotting. Once their secretion is confirmed, these osteogenic factors are incubated with mouse primary OBs under osteogenic culture conditions and their osteogenic potential will be assessed. As a negative control, GFP-encoding scAAV9 serotype is used.

Next, whether AAV-mediated gene addition of secretory osteogenic factors prevent bone loss in the setting of osteoporosis is investigated. Sham or OVX surgery is conducted in 4-months-old female mice (C57BL/6J, n=12 mice/group) and 4 weeks after surgery, a single dose of 1×1010 genome copies of GFP-encoding scAAV9 serotypes that express PTH, PTHrP, or DJ-1 (PTH-scAAV9, PTHrP-scAAV9, D.J-1-scAAV9) are administered into the quadriceps muscle of hindlimbs via IM injection. As a negative control, GFP-encoding scAAV9 vector control (cont-seAAV9) is administered into these mice. Alternatively, these AAV serotypes are IM injected into 22-months-old female mice (C57BL/6J, n=12 mice/group). Two months after treatment, mice are labeled with calcein and alizarin red and subjected to dynamic histomorphometry in order to assess in vivo OB and OC activities. For skeletal analysis. microCT, TRAP staining (OC differentiation), and IHC for Coll and Runx2 (OB differentiation) are performed in long bones and vertebrae. Lastly, serum levels of CTX (bone resorption), PINP and BSAP (bone formation), and calcium and phosphorus (mineral homeostasis) are measured by ELISAs or colorimetric analysis. This analysis is accompanied by qPCR analysis of bone RNA measuring expression of OB differentiation genes. Lastly, serum levels of transgenes (PTH, PTHrP, or DJ-1) are measured by ELISAs.

### Example 3 Inducible Deletion of Shn3 in Osteoblasts Promotes Bone Formation in Adult Mice

To examine the effects of short-term inhibition of SHN3 on bone formation, an inducible, osteoblast-specific Shn3-knockout mice were generated by crossing Shn3^{fl/fl} mice with osteocalcin-CreERT mice expressing a tamoxifen-induced Cre recombinase in mature osteoblasts (Shn3^{Ocn-Ert}). These mice were further crossed with the Cre reporter Rosa^{mT/mG} mice to visualize Cre-expressing cells (Shn3^{Ocn-Ert};Rosa^{mT/mG}). Treatment of Shn3^{Ocn-Ert}:Rosa^{mT/mG}mice with tamoxifen resulted in the expression of GFP in mature osteoblasts at the surface of trabecular and cortical bones, indicating osteoblast-specific deletion of Shn3 (FIG. 23). Accordingly, these mice showed a significant increase in trabecular bone mass compared to tamoxifen-treated control mice (FIG. 23). These results indicate that inducible deletion of Shn3 in mature osteoblasts is sufficient to increase bone mass in adult mice.

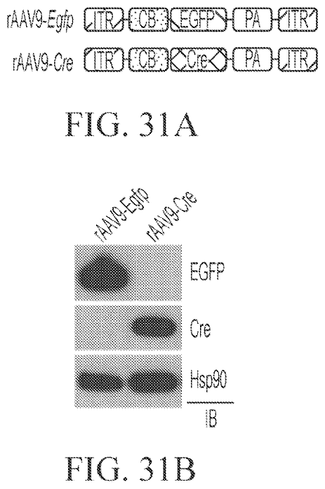

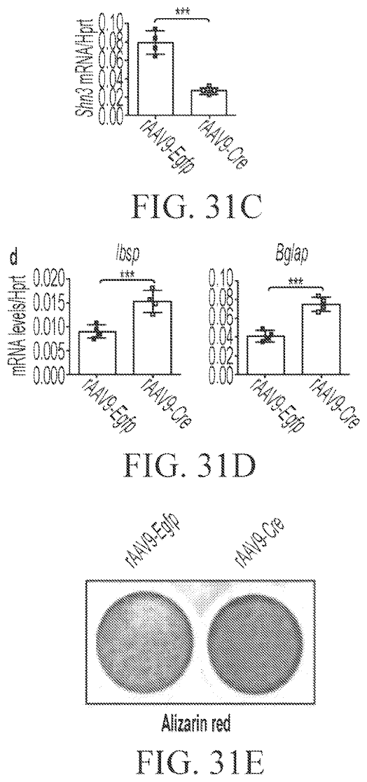

Cre-encoding scAAV9 vector (scAAV9-Cre, FIG. 30) were generated to serve as a facilitator for Cre-recombination in Shn3^{fl/fl} mice. The Shn3^{fl/fl} mice were first treated scAAV9-Cre in cultured COBs isolated from Shn3^{fl/fl} mice (FIG. 30), and as expected, scAAV9-mediated Cre expression in Shn3^{fl/fl} COBs resulted in the deletion of Shn3 and enhanced osteoblast differentiation (FIG. 31). Next, scAAV9-Cre was injected into two-month-old Shn3^{fl/fl};Rosa^{mT/mG}mice via i.v. administration. Two months after injection, expression of Cre mRNA in the femur (FIG. 31) and Cre-mediated expression of EGFP protein in osteoblast lineage cells residing on the bone surface (FIG. 30) were validated by RT-PCR and fluorescence microscopy, respectively. Compared to scAAV9-EGFP treated femurs, scAAV9-Cre-treated femurs showed a significant decrease in shn3 mRNA levels (FIG. 31) and an increase in relative trabecular bone mass (FIG. 31). These results demonstrate that systemically delivered scAAV9-Cre in Shn3^{fl/fl} mice targets osteoblast lineage cells and mediates Shn3 deletion to increase bone mass, Importantly, these results demonstrate the potency of scAAV9-mediated transgene delivery to osteoblast lineage cells to dramatically alter bone physiology.

### Example 4: Silencing of Shn3 by scAAV9 Carrying the amiR-shn3 Transgene Promotes Bone Formation in Vivo

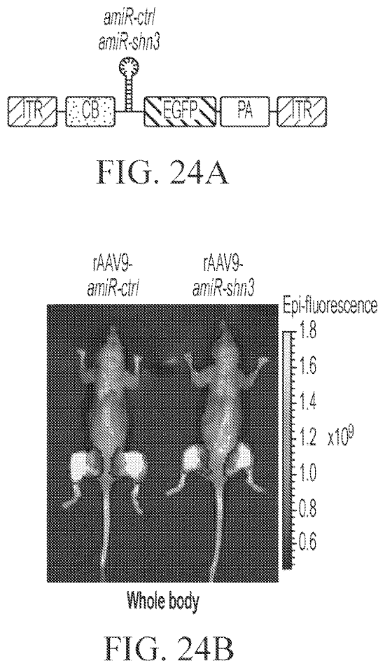

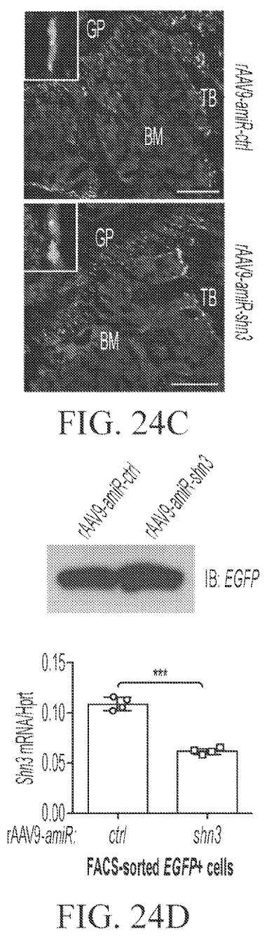

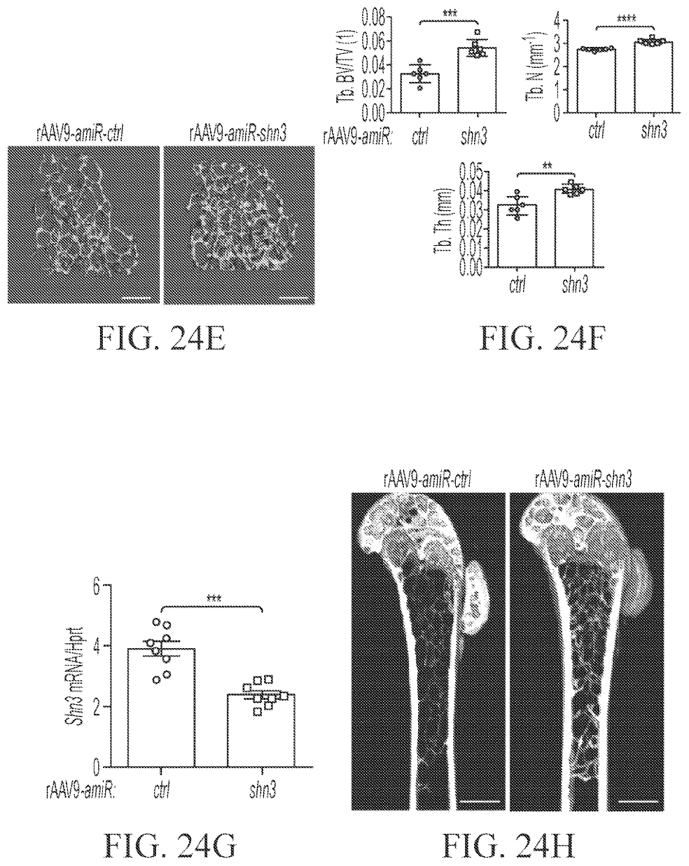

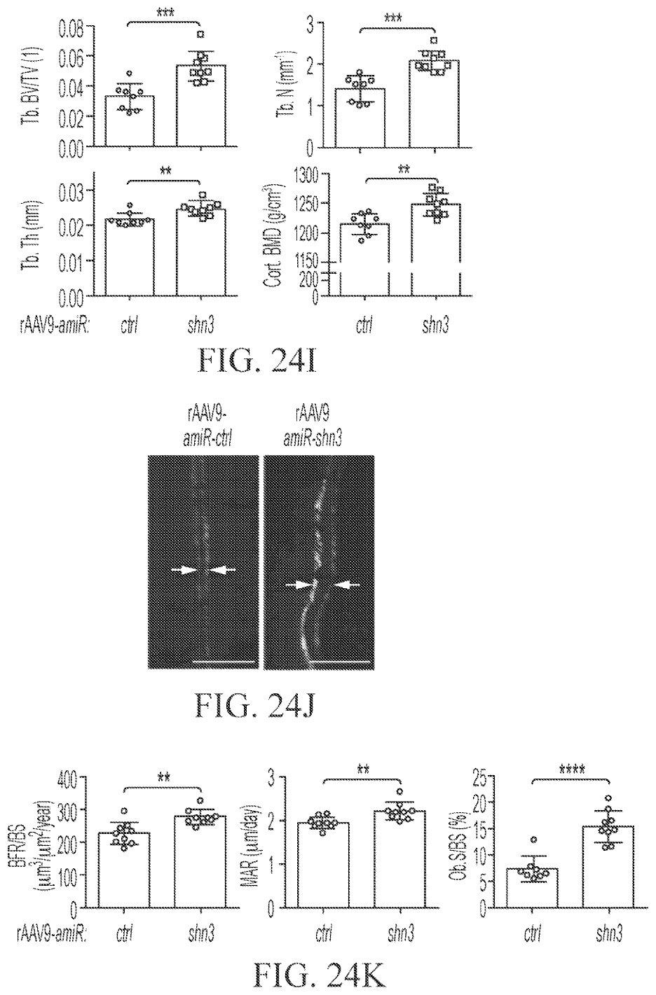

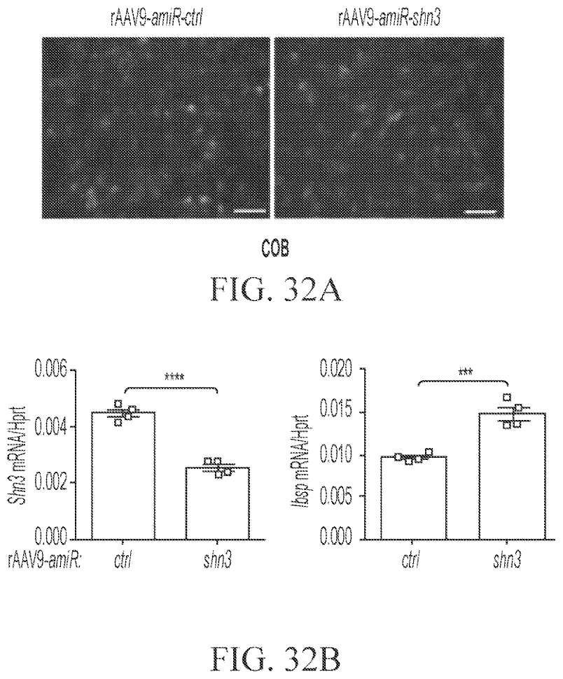

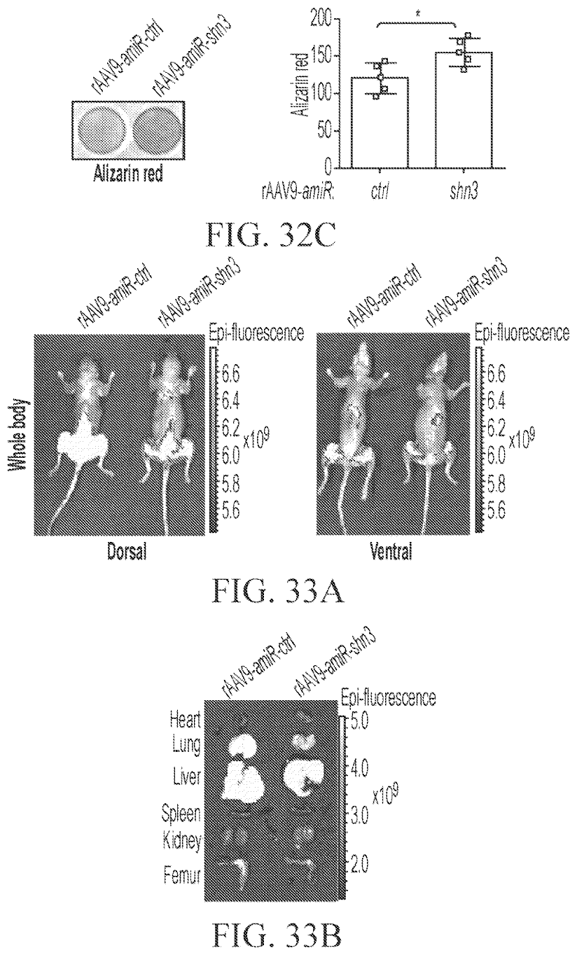

Artificial microRNAs (amiRs), siRNA cassettes designed into miRNA scaffolds have been shown to efficiently promote target message degradation in vivo. In addition, such designs allow expression cassettes to be driven by pol II promoters, expanding the ability to control gene knockdown with scAAV vectors. Artificial microRNA cassettes were engineered to target shn3 (amiR-shn3) or a control (amiR-ctrl). In this design, the amiR is inserted intronically between the CB promoter and the Egfp reporter gene (FIG. 24), which allows for visual tracking of positively transduced cells or tissues. The test cassettes were then packaged into AAV9 capsids. Treatment with scAAV9-amiR-shn3 or scAAV9-amiR-ctrl resulted in positively transduce COBs in vitro (FIG. 32). Compared to amiR-ctrl-treated COBs, treatment with scAAV9-amiR-shn3 resulted in ˜50% reduction of shn3 mRNA levels, and relative increases in ibsp expression and mineralization (FIG. 32).

To examine the ability of amiRs to enhance bone anabolic activity in vivo, the scAAV9-amiR-shn3 vector was injected via i.a. administration into the knee joints of two-month-old mice. Two months following treatment, EGFP expression in hindlimbs and femurs was examined by IVIS optical imaging (FIG. 24) and fluorescence microscopy (FIG. 24), respectively. Importantly, FACS-sorted EGFP-expressing cells isolated from the femur showed ˜50% reduction of shin3 mRNA levels (FIG. 24). Compared to amiR-ctrl-treated femurs, amiR-shn3-treated femurs showed a significant increase in relative trabecular bone mass (FIG. 24). These results demonstrate that local delivery of scAAV9-amiR-shm3 is effective in knocking down SHN3 expression in osteoblast lineage cells and in turn increases bone mass in vivo.

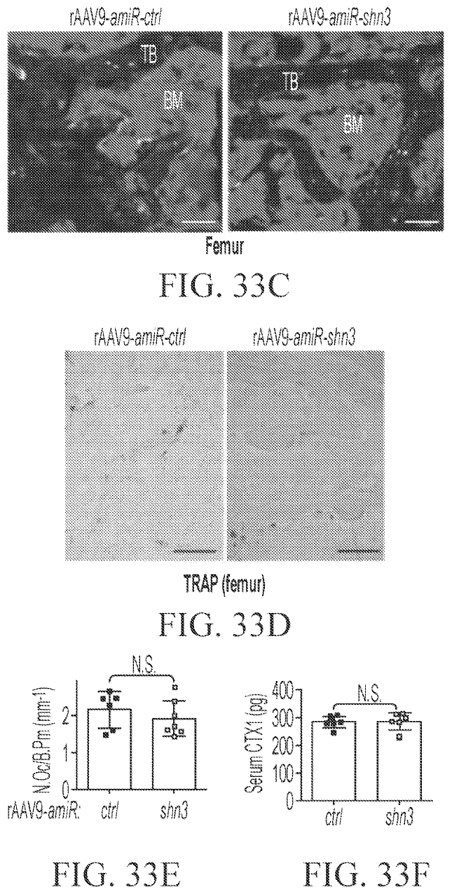

The ability of scAAV9-amiR-shn3 could promote in vivo bone anabolic activity following systemic delivery was then examined. Two months after i.v. administration into three-month-old mice, EGFP expression was predominantly detected in the hindlimbs, liver, and femurs as expected (FIG. 33). Femurs transduced by scAAV9-amiR-shn3 displayed ˜50% reduction of shn3 mRNA levels (FIG. 24) and a significant increase in trabecular bone mass and cortical bone mineral density (FIG. 24). Likewise, in vivo osteoblast activity was increased in these mice, as shown by greater bone formation rate (BFR), mineral apposition rate (MAR), and osteoblast surface per bone surface (Ob.S/BS) (FIG. 24). However, the number of tartrate-resistant acid phosphatase (TRAP)-positive osteoclasts and serum levels of the bone resorption marker C-terminal telopeptide type I collagen (CTX) were unchanged in these mice (FIG. 33). These results demonstrate that systemically delivered AAV9-amiR-shn3 reduced shn3 expression in osteoblast lineage cells, augmented osteoblast activity, and increased bone mass without any alteration in osteoclast number and function in vivo. Thus, the scAAV9-amiR-shn3 vector may be useful for the treatment of osteoporosis as a potent bone anabolic agent.

### Example 4: scAAV9-Mediated Silencing of Shn3 Counteracts Bone Loss in Postmenopausal Osteoporosis

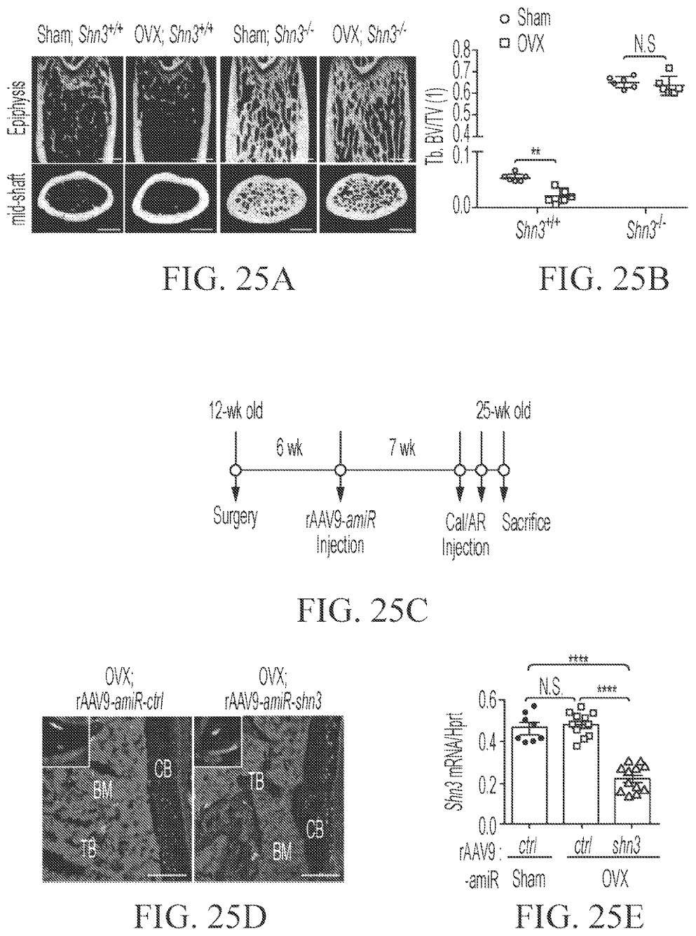

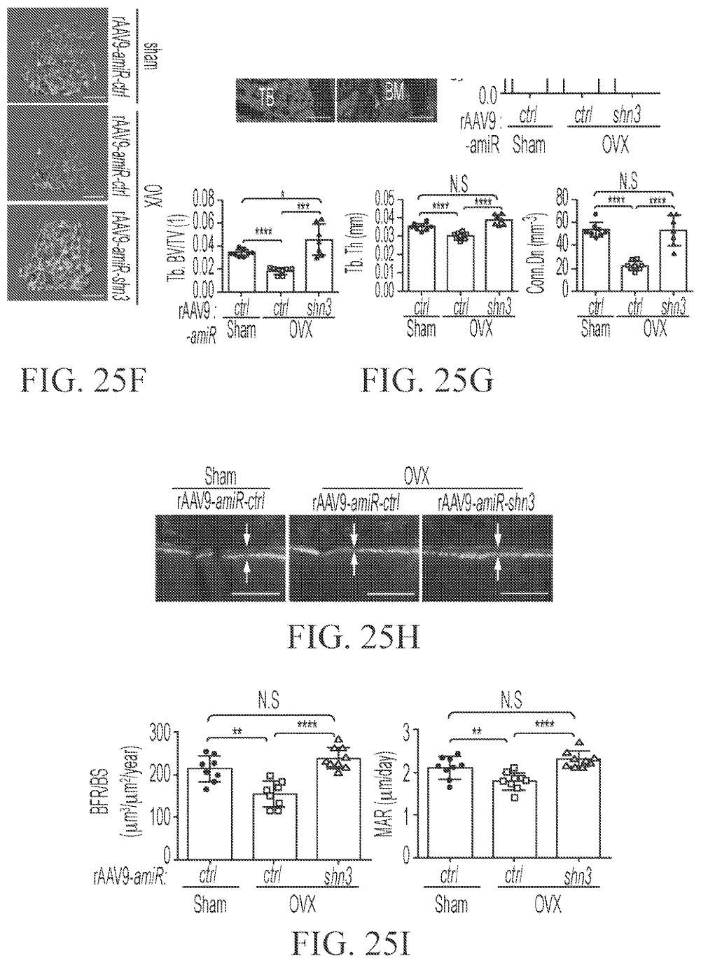

Inhibition of Wnt antagonists has been recognized as a new approach for therapeutic intervention in patients with osteoporosis. Previous studies identified SHN3 as an inhibitor of osteoblast differentiation via perturbation of Wnt signaling. Ovariectomized (OVX) mice are an established models for postmenopausal osteoporosis. To further establish that inhibition of Shn3 may be an attractive target to promote bone formation as a therapy for osteoporosis, three-month-old female mice lacking SHN3 (Shn3) were subjected to ovariectomies, and bone mass was assessed by microCT two months post-surgery. While OVX surgery induced a significant reduction in trabecular bone mass in WT mice, OVX-induced bone loss was completely prevented by Shn3 deletion, as trabecular bone mass was comparable between sham-Shn3^{-/-} mice and OVX-Shn3^{-/-} mice (FIG. 25). Thus, targeting Shn3 has therapeutic potential to prevent bone loss in postmenopausal osteoporosis.

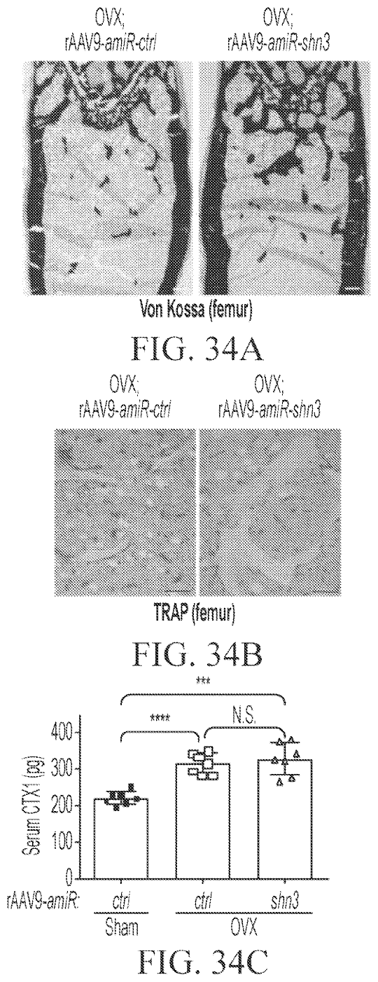

To test the therapeutic effects of scAAV9-amiR-shn3 in postmenopausal osteoporosis, sham or OVX surgery was conducted on three-month-old wild-type female mice, and vector was i.v. injected six weeks post-surgery (FIG. 25). Seven weeks later, treated femurs showed efficient transduction, leading to ˜50% knockdown of shn3 (FIG. 25). As expected, amiR-ctrl-expressing OVX mice showed a significant decrease in trabecular bone mass relative to sham mice. However, when treated with scAAV9-amiR-shn3, bone loss was completely reversed in the femur of OVX mice, as shown by greater trabecular BV/TV, thickness, and connectivity density (FIG. 25 and FIG. 34). Likewise, femoral BFR and MAR were increased in these mice relative to amiR-ctrl-expressing OVX mice, demonstrating enhanced osteoblast activity in vivo (FIG. 25). Notably, shn3 silencing by scAAV9-amiR-shn3 does not alter osteoclast function in vivo as the number of TRAP-positive osteoclasts and bone resorption activity are comparable between OVX mice expressing amiR-ctrl and amiR-shn3 (FIG. 34). Finally, biomechanical testing showed that the strength and stiffness of femurs were considerably protected from OVX-induced bone loss of mice treated with scAAV9-amiR-shn3 (FIG. 25), indicating that scAAV9-mediated silencing of shn3 improves clinically meaningful endpoints in osteoporotic mice. Taken together, these results demonstrate that systemically delivered scAAV9-amiR-shn3 promotes bone formation and enhances clinically relevant mechanical properties of bone after the onset of OVX-induced osteoporosis,

### Example 5: Grafting of a Bone-Targeting Peptide Motif onto the AAV9 Capsid Improves Bone-Specific Transduction

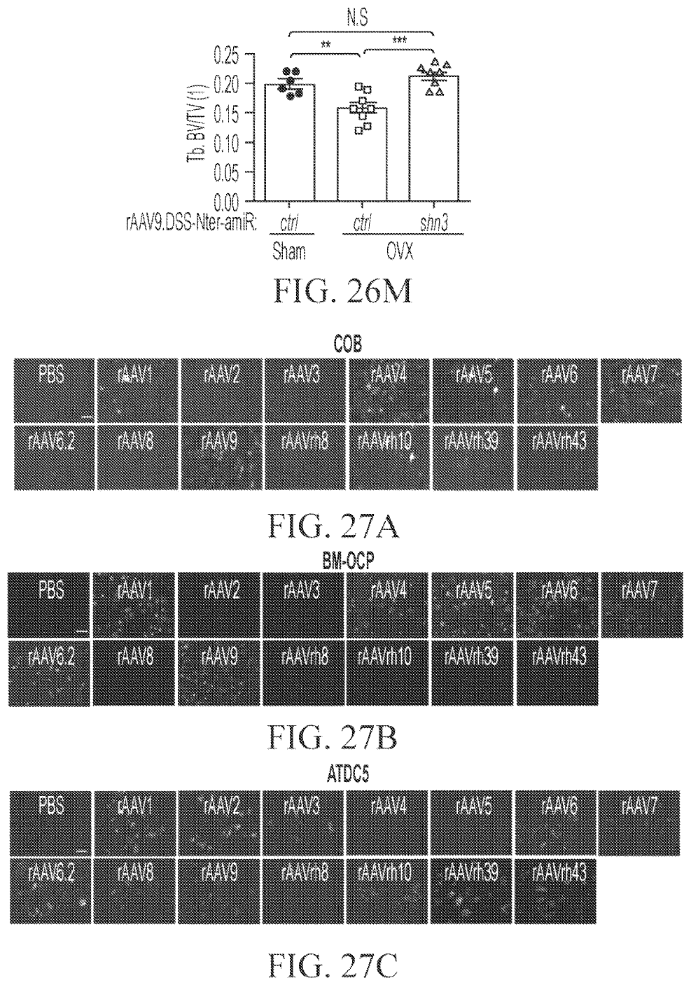

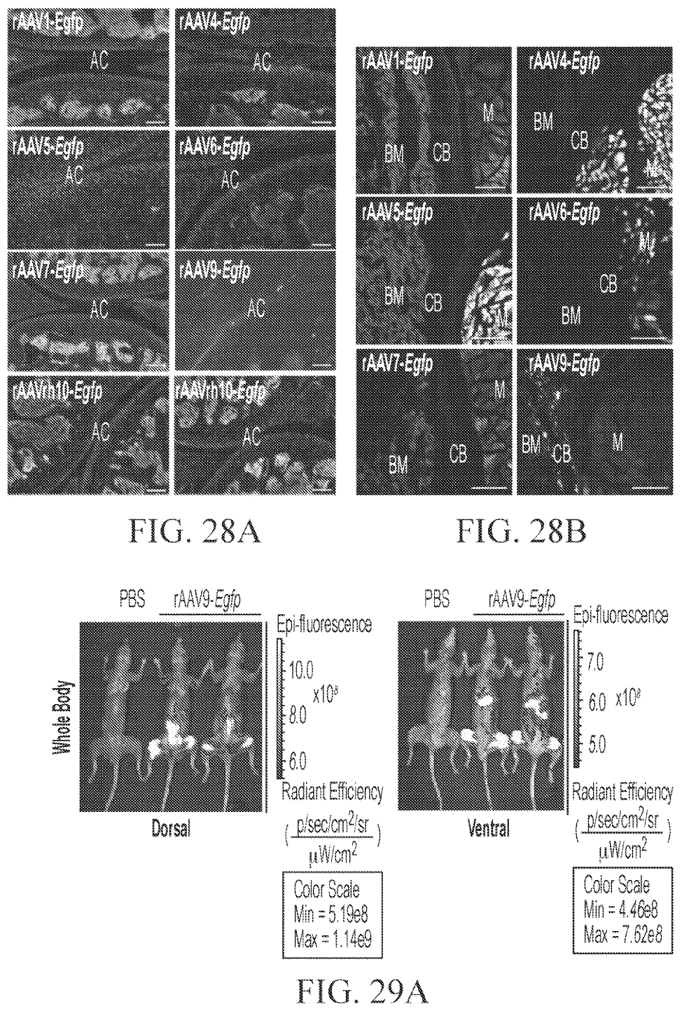

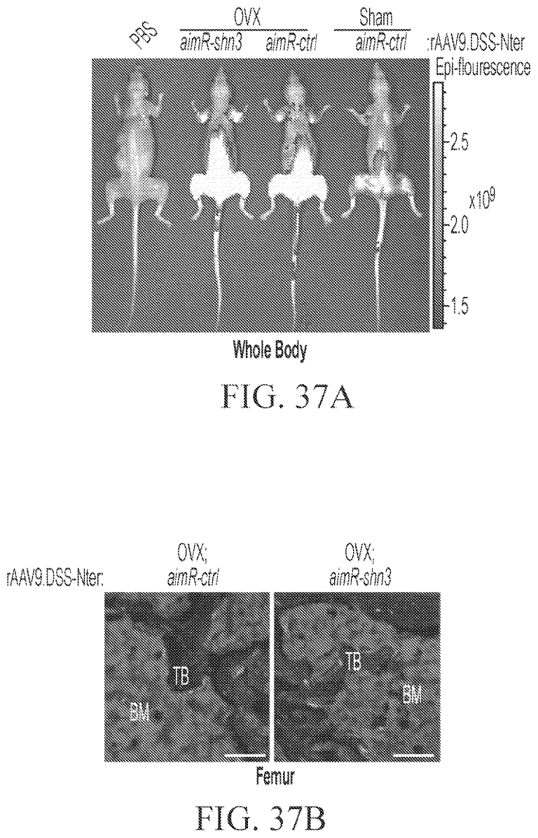

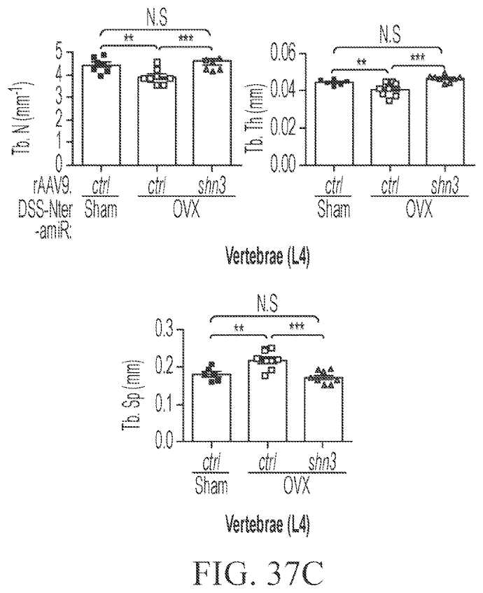

The capacity for the new AAV9. DSS-Nter bone-tropic capsid to deliver the therapeutic amiR-shn3 transgene (scAAV9. DSS-Nter-amiR-sha3) to OVX mice was examined. Sham or OVX surgery was conducted on three-month-old female mice and scAAV9. DSS-Nter-amiR-shn3 was i.v. injected six weeks post-surgery. Seven weeks after injection, animals exhibit strong EGFP expression throughout the hind section of the animals (FIG. 37) and in osteoblast lineage cells of the femurs (FIG. 37), indicating robust transduction. Levels of shn3 mRNAs were markedly reduced in amik-shn3-expressing OVX femurs relative to amiR-ctrl-expressing sham or OVX femurs (FIG. 27). While amiR-ctrl-expressing OVX mice showed a significant reduction in trabecular bone mass relative to sham mice, bone loss was completely reversed in the femurs (FIG. 27) and lumbar vertebrae (FIG. 27, FIG. 37) of OVX mice treated with scAAV9. DSS-Nter-amiR-shn3, as shown by the greater trabecular BV/TV, thickness, number, and connectivity density. Taken together, these results demonstrate that delivery of amiR-sha3 by the bone-tropic AAV9. DSS-Nter capsid can counteract bone loss in postmenopausal osteoporosis.

### Example 6: Inhibition of Bone Loss in Human Patients and Animal Models of Osteoporosis

A humanized biologic antibody against the receptor activation of NF-κβ (RANK, TNFRSF11A) ligand interferes with the interaction between RANK ligand (RANKL) and RANK, which inhibits the RANK signaling required for osteoclast differentiation. Thus, its treatment prevents bone loss in human patients and animal models with osteoporosis by suppressing osteoclast-mediated bone resorption.

The capacity for the new AAV9. DSS-Nter bone-tropic capsid to deliver a therapeutic amiR-33-mRANKi transgenes (scAAV9. DSS-Nter-amiR-mRANKi 1/2) is being examined for suppression of osteoclast differentiation by silencing RANK in osteoclast precursors, thereby inhibiting bone loss in human patients and animals models of osteoporosis.

### Methods

### scAAV Vector Design and Production

DNA sequences for amiR-33-ctrl and amiR-33-shn3 were synthesized as gBlocks and cloned into the intronic region of the pAAVsc-CB6-Egfp plasmid at the restriction enzyme sites (PstI and BgIII). The pAAVsc-CB6-Cre vector was generated by replacing the Eg/p reporter with Cre recombinase. Constructs were verified by sequencing. Previous studies indicated that doxycycline-inducible expression of shR.NA targeting mouse Shn3 in transgenic mice resulted in a decrease in shn3 mRNA levels and a relative increase in bone mass. The same targeting sequence was used to generate the amiR-33-shin3 cassette. The pAAV-anuR-ctrl and pAAV-amiR-shn3 constructs were packaged into AAV9 capsids. Additionally, the pAAVsc-CB6-Egfp construct was packaged into AAV1, AAV2, AAV3, AAV4, AAV5, AAV6. AAV6.2, AAV7, AAV8, AAV9, AAV9-HR, AAVrh8, AAVrh 10, AAVrh39, and AAVrh43 capsids. scAAV production was performed by transient transfection of HEK293 cells, purified by CsCl sedimentation, titered by droplet digital PCR (ddPCR) on a QX200 ddPCR system (Bio-Rad) using the Egfp prime/probe set as previously described. The sequences of gBlocks and oligonucleotides for ddPCR and are listed in Table 3.

TABLE 3
Sequences of primers, probes, and gblocks

| Gene | Forward | SEQ ID NO: | Reverse | SEQ ID NO: |
|---|---|---|---|---|
| Mouse | AGAGGCCATTCAGAC | 35 | CTGCGGAAGCTGAG | 36 |
| Shn3 | GAGTGT | | AGATGT | |
| Mouse | CACAATATCAAGGAT | 37 | ACATCAGTTCTGTTC | 38 |
| Alp | ATCGACGTGA | | TTCGGGTACA | |
| Mouse | TACAAACCATACCCA | 39 | AGTGCTCTAACCACA | 40 |
| Runx2 | GTCCCTGTTT | | GTCCATGCA | |
| Mouse | CAGGGAGGCAGTGAC | 41 | AGTGTGGAAAGTGT | 42 |
| Bsp | TCTTC | | GGCGTT | |
| Mouse | ATGGCGTCCTCTCTGC | 43 | GAAGGGTGGGTAGT | 44 |
| Osx | TTGA | | CATTTG | |
| Mouse | GCAGCACAGGTCCTA | 45 | GGGCAATAAGGTAG | 46 |
| Ocn | AATAG | | TGAACAG | |
| Mouse | CTGTCCCAACCCCCA | 47 | ACGTATTCTTCCGGG | 48 |
| Colla1 | AAG | | CAGAA | |
| Mouse | CTGGTGAAAAGGACC | 49 | CCAGTTTCACTAATG | 50 |
| Hprt | TCTCGAAG | | ACACAAACG | |
| EGFP | AGCAAAGACCCCAAC | 51 | GGCGGCGGTCACGA | 52 |
| | GAGAA | | A | |

| | | |
|---|---|---|
| EGFP- | 6FAM-CGCGATCACATGGTCCTGCTGG-TAMRA | 53 |
| probe | | |
| amiR- | TTTGTCTTTTATTTCAGGTCCCAGATCTAGGGCTCTGCGTTT | 54 |
| 33-ctrl | GCTCCAGGTAGTCCGCTGCTCCCTTGGGCCTGGGCCCACTG | |
| (amiR- | ACAGCCCTGGTGCCTCTGGCCGGCTGCACACCTCCTGGCGG | |
| ctrl) | GCAGCTGTGTACAAACTACTTGAGAGCAGGTGTTCTGGCAA | |
| | TACCTGCCTGCTCTGTAATAGTTTGTACACGGAGGCCTGCCC | |
| | TGACTGCCCACGGTGCCGTGGCCAAAGAGGATCTAAGGGCA | |
| | CCGCTGAGGGCCTACCTAACCATCGTGGGGAATAAGGACAG | |
| | TGTCACCCCTGCAGGGGATCCGGTGGTGGTGCAAATCA | |

### Generation of bone-Targeting scAAV9 Vector

The DNA sequence encoding the bone-targeting peptide motif DSS (AspSerSer)₆ (SEQ ID NO: 16) was codon-optimized. To generate the Q588-DSS-A589 capsid (DSS-588), a plasmid expressing AAV2 rep gene and AAV9 cap gene (pAAV2/9) was modified to insert the DSS sequence into the AAV9 cap gene between the Q588 and A589 codons (pAAV2/9. Q588-DSS-A589). This plasmid was used in scAAV production. To generate the DSS-Nter capsid, a pair of plasmids were used. First, the start codon of VP2 in pAAV2/9 was mutated (ACG->ACC), so that only VP1 and VP3 are expressed (pAAV2/9. noVP2). In another plasmid, the DSS sequence was fused to the N-terminus of the AAV9 VP2 ORF. A Kozak sequence and ATG start codon were placed immediately upstream of the DSS sequence allowing for optimal expression driven by the CMV promoter [pcDNA.DSS-VP2 (AAV9)]. The plasmids pAAV2/9.noVP2 and pcDNA.DSS-VP2 (AAV9) were used in scAAV production.

### Cells

The chondrogenic ATDC5 cells were purchased from Sigma and cultured in DMEM/Ham's F12 medium supplemented with 2% FBS, 2 mM L-glutamine and 1% penicillin/streptomycin. Additionally, primary osteoprogenitors (COB) were isolated from calvaria of five-day-old wild type neonates (C57BL/6J) using Collagenase type II (50 mg/ml, Worthington, LS004176) and Dispase II (100 mg/ml, Roche, 10165859001) and were maintained in α-MEM medium (Gibco) containing 10% FBS (Gibco), 2 mM L-glutamine (Corning), 1% penicillin/streptomycin (Coming), and 1% nonessential amino acids (Corning). COBs were differentiated with ascorbic acid (200 UM, Sigma, A8960) and β-glycerophosphate (10 mM, Sigma, G9422). Finally, bone marrow cells were flushed from the femurs and tibias of two-month-old mice (C57BL/6J), and cultured in petri dishes in a-MEM medium with 10% FBS and 20 ng/ml of M-CSF (R&D systems) to obtain bone marrow-derived osteoclast precursors (BM-OCP). 12 hours later, nonadherent cells were re-plated into tissue culture dishes and cultured in the same mediom for three days. BM-OCPs then differentiated into osteoclasts in the presence of RAMKL (20 ng/ml; R&D systems) and M-CSF (20 ng/ml; R&D systems) for six days.

### Mice

Shn3^{-/-} mice and Shn3/2 mice were generated as previously described and maintained on BALB/cJ and C57BL/6J background, respectively. Osteocalcin-ERT/Cre mice with tamoxifen-induced Cre recombinase expression in mature osteoblasts were crossed with Shn3^{fl/fl} mice to obtain Shn3^{fl/fl};Och-ERT/Cre mice. To label Cre-expressing cells, Shn3^{fl/fl};Och-ERT/Cre mice were further crossed with Rosa^{mT/mG} ere reporter mice. For postnatal activation of ERT/Cre, 100 mg/kg tamoxifen (Sigma) in corn oil (Sigma) was intraperitoneally injected to 2-month-old female mice once a day for 5 consecutive days. Mouse genotypes were determined by PCR on tail genomic DNA. Primer sequences are available upon request. Control littermates were used and analyzed in all experiments.

### MicroCT Analysis

MicroCT was used for qualitative and quantitative assessment of trabecular and cortical bone microarchitecture and performed by an investigator blinded to the genotypes of the animals under analysis. Femurs excised from the indicated mice were fixed with 10% neutral buffered formalin and scanned using a microCT 35 (Scanco Medical) with a spatial resolution of 7 μm. For trabecular bone analysis of the distal femur, an upper 2.1 mm region beginning 280 μm proximal to the growth plate was contoured. For cortical bone analysis of femur, a midshaft region of 0.6 mm in length was used. MicroCT scans of L4 spinal segments were performed using isotropic voxel sizes of 12 μm. 3D reconstruction images were obtained from contoured 2D images by methods based on distance transformation of the binarized images. Alternatively, the Inveon multimodality 3D visualization program was used to generate fused 3D viewing of multiple static or dynamic volumes of microCT modalities (Siemens Medical Solutions USA, Inc). All images presented are representative of the respective genotypes (n>5).

### Histology, histomorphometry, and immunofluorescence

For histological analysis, femurs and vertebrae were dissected from the mice treated with rAAVs vectors, fixed in 10% neutral buffered formalin for two days, and decalcified by 5% tetrasodium EDTA for 2-4 weeks. Tissues were dehydrated by passage through an ethanol series, cleared twice in xylene, embedded in paraffin, and sectioned at a thickness of 6 μm along the coronal plate from anterior to posterior. Decalcified femoral sections were stained with hematoxylin and eosin (H&E) or tartrate-resistant acid phosphatase (TRAP).

For dynamic histomorphometric analysis, 25 mg/kg calcein (Sigma, C0875) and 50 mg/kg alizarin-3-methyliminodiacetic acid (Sigma, A3882) dissolved in 2% sodium bicarbonate solution were subcutaneously injected into mice at six day-interval. After fixed in 10% neutral buffered formalin for two days, undecalcified femur samples were embedded in methylmethacrylate and proximal metaphysis is sectioned longitudinally (5 μm) and stained with McNeal's trichrome for osteoid assessment, toluidine blue for osteoblasts, and TRAP for osteoclasts. A region of interest is defined and bone formation rate/bone surface (BFR/BS), mineral apposition rate (MAR), bone surface (BS), osteoblast surface (Ob.S/BS), and osteoclast surface (Oc.S/BS) are measured using a Nikon Optiphot 2 microscope interfaced to a semiautomatic analysis system (Osteometrics). Measurements were taken on two sections/sample (separated by ˜25μm) and summed prior to normalization to obtain a single measure/sample in accordance with ASBMR standards. This methodology has undergone extensive quality control and validation and the results were assessed by two different researchers in a blinded fashion.

For immunofluorescence, fresh femurs and vertebrae dissected from rAAV-treated mice were collected and immediately fixed in ice-cold 4% paraformaldehyde solution for two days. Semi-decalcification was carried out for five days in 0.5 M EDTA pH 7.4 at 4° C. with constant shaking (age≥1 week), and infiltration was followed with a mixture of 20% sucrose phosphate buffer for one day and with 25% sucrose phosphate buffer next day. All samples were embedded in 50/50 mixture of 25% sucrose solution and OCT compound (Sakura) and cut into 12-um-thick sagittal sections using a cryostat (Leica). Immunofluorescence staining and analysis was performed as described previously. Briefly, after treatment with 0.2% Triton X-100 for 10 min, sections were blocked with 5% donkey serom at room temperature for 30 min and incubated overnight at 4°° C. with anti-BGLAP antibody (sc-365797, Santa Cruz, 1:150). Primary antibodies were visualized with donkey anti-rat IgG Alexa-594 (1:500, Molecular Probes). Nuclei were counterstained with DAPI. An Olympus IX81 confocal microscope or Leica TCS SP5 II Zeiss LSM-880 confocal microscope was used to image samples.

### Biomechanical Analysis

Femora were mechanically tested in three-point bending using an electrical force mechanical testing machine (Electroforce 3230, Bose Corporation, Eden Prairie, MN) at the Center for Skeletal Research Imaging and Biomechanical Testing Core. The bending fixture had a bottom span length of 8 mm. The test was performed with the load point in displacement control moving at a rate of 0.05 mm/sec with force and displacement data collected at 60 Hz. All of the bones were positioned in the same orientation during testing with the cranial surface resting on the supports and being loaded in tension. Bending rigidity (EI, N-mm2), apparent modulus of elasticity (Eapp, MPa), ultimate moment (Mult, N-mm), apparent ultimate stress (σapp, MPa), work to fracture (Wfrac, mJ), and apparent toughness (Uapp, mJ/mm3) were calculated based on the force and displacement data from the tests and the mid-shaft geometry measured with microCT. Work to fracture is the energy that that was required to cause the femur to fracture, and it was calculated by finding the area under the force-displacement curve using the Riemann Sum method. Bending rigidity was calculated using the linear portion of the force- displacement curve. The minimum moment of inertia (Imin) was used when calculating the apparent modulus of elasticity.

### ELISA Analysis

CTX1 ELISA (Abclonal MC0850) analysis was performed by using a kit according to the manufacturer's instructions.

### Osteoblast Differentiation Analysis

For alkaline phosphatase (ALP) staining, osteoblasts were fixed with 10% neutral formalin buffer and stained with the solution containing Fast Blue (Sigma, FBS25) and Naphthol AS-MX (Sigma, 855). Alternatively, osteoblasts were incubated with 10-fold diluted Alamar Blue solution (Invitrogen, DAL1100) for cell proliferation. Subsequently, cells were washed and incubated with a solution containing 6.5 mM Na₂CO₃, 18.5 mM NaHCO₃, 2 mM MgCl₂, and phosphatase substrate (Sigma, S0942), and ALP activity was measured by laminometer (Biorad).

To assess extracellular matrix mineralization in mature osteoblasts, cells were washed twice with phosphate-buffered saline (PBS) and fixed in 70% EtOH for 15 min at room temperature. Fixed cells were washed twice with distilled water and then stained with a 2% alizarin red solution (Sigma, A5533) for 5 min. Cells were then washed three times with distilled water and examined for the presence of calcium deposits. Mineralization was quantified by the acetic acid extraction method.

### Quantitative RT-PCR Analysis

Total RNA was purified from cells using QIAzol (QIAGEN) and cDNA was synthesized using the High-Capacity cDNA Reverse Transcription Kit from Applied Biosystems. Quantitative RT-PCR was performed using SYBR@ Green PCR Master Mix (Bio-Rad) with CFX connect RT-PCR detection system (Bio-Rad). To measure Sha3 mRNA levels in bone tissues, after removal of bone marrow, tibias were snap-frozen in liquid nitrogen for 30 sec and in turn homogenized in 1 ml of QIAzol for 1 min.

Alternatively, femurs and tibias dissected from rAAV9-treated mice were crushed in Hanks Balanced Salt Solution (Life Technologies) containing 10 mM HEPES (pH 7.2) (CellGro) and enzymatically digested with 2.5 mg/ml. Collagenase A (Roche) and 1 unit/mL. Dispase II (Roche) for 15 min at 37° C. under gentle agitation. The resulting cell suspensions were filtered (40 μm), washed using PBS (pH 7.2) containing 0.5% BSA (Fraction V) and 2 mM EDTA. After washing, cells were resuspended in PBS (pH 7.2) with 2 mM EDTA and 1 μg/mL 4-6, Diamidino-2-Phenylindole (DAPI) (live/dead exclusion) and EGFP-expressing cells were sorted using a FACS Aria II SORP cell sorter (Becton Dickinson) with exclusion of DAPI+cells and doublets. Total RNA was purified from cells using QIAzol. Primers used for PCR are described in Table 3.

### Imnunoblotting Analysis

Cells were lysed in TNT lysis buffer (50 mM Tris-HCI (pH 7.4), 150 mM NaCl, 1% Triton X-100, 1 mM EDTA, 1 mM EGTA, 50 mM NaF, 1 mM Na₃VO₄, 1 mM PMSE and protease inhibitor cocktail (Sigma)) and protein amounts from cell lysates were measured using DC protein assay (Bio-Rad). Equivalent amounts of proteins were subjected to SDS-PAGE, transferred to Immunobilon-P membranes (Millipore), immunoblotted with anti-GFP antibody

(JL-8, 632381, Takara, 1:1000), anti-Cre recombinase antibody (ab24607, Abcam, 1:1000), anti-Hsp90 antibody (675402, Biolegend, 1:1000), and developed with ECL (Thermo fisher scientific). Immunoblotting with anti-HSP90 antibody was used as a loading control. Alternatively, dissected femurs and soft tissues were homogenized in RIPA lysis buffer (89900, Thermo fisher scientific) and tissue lysates were subjected to immunoblotting analysis.

### Screening of rAAV Serotypes that Transduce Osteoblasts, Osteoclasts, or Chondrocytes in Vitro

ATDCS cells or primary COBs were plated at a density of 1×10⁴ cells/well in 24-well plate and 24 hrs later, they were incubated with AAV1, rAAV2, rAAV3, rAAV4, TAAV5, AAV6, rAAV6.2, rAAV7, rAAV8, rAAV9, IAAVrh8, rAAVrh10, rAAVrh39, or rAAVrh43 vectors packaging the CB-Egfp reporter transgene at three different titers (10⁹-10¹¹/mL genome copies). 48 hrs later, cells were washed with PBS and EGFP expression was monitored by the EVOS PL imaging system (Thermo fisher scientific). Alternatively, cells were lysed in TNT lysis buffer and BOPP expression was assessed by immunoblotting with anti-EOFP antibody and quantified using Image J software. Lastly, primary bone marrow monocytes were plated at a density of 5×10⁵ cells/well in 24-well plates and cultured in the presence of 10 ng/ml of RAMKL and 20 ng/ml of M-CSF for two days to differentiate to osteoclast precursors. Three days after treatment with TAAV-Egfp vectors, EGFP expression was assessed by EVOS FL imaging system and by immunoblotting with anti-EGFP antibody.

For screening rAAV vectors in vivo, 10 pil of rAAV-Egfp vectors (1×10¹¹ GC; 5×10¹² (C/kg) was intraarticularly (i.a.) injected into knee joints of two-month-old male mice (Jackson Laboratory). Two weeks after injection, femurs and knee joints were dissected for IVIS-100 optical imaging or cryo-sectioning.

### Effects of rAAV9-Mediated Delivery of Cre-Recombinase or amiR-shn3 on Bone Formation

For a local delivery, 10 pil of rAAV9 carrying amiR-ctrl or amiR-shn3 (1×10¹¹ GC; 5×10¹² GC/kg) was i.a. injected into knee joints of two-month-old male mice. Two months after injection, femurs were dissected for microCT analysis.

For a systemic delivery, 200 pl of xAAV9 carrying Egfp, Cre, amiR-ctrl or amiR-shn3 (4×10¹¹ GC: 2×10¹³ GC/kg) was intravenously (i.v.) injected into mice and two months later, mice were subcutaneously injected with calcein and alizarin-3-methyliminodiacetic acid at six day-interval for dynamic histomorphometric analysis. Non-labeled mice were used to monitor EGFP expression using the IVIS-100 optical imaging or cryo-sections.

### Therapeutic Effects of Systematically Delivered rAAV9-amiR-shn3 in a Mouse Model of Postmenopausal Osteoporosis

Mouse models of postmenopausal osteoporosis were generated by anesthetizing and bilaterally ovariectomizing (OVX) three-month-old female mice (Jackson Laboratory). Six weeks after the surgery, sham or OVX mice were i.v. injected with 200 μl of rAAV9 or rAAV9. DSS-Nter carrying amiR-ctrl or amiR-shn3 (4×10¹¹ ) GC; 2×10¹³ GC/kg). Mice were randomly divided into six groups with rAAV9 or rAAV9. DSS-Nter: sham+xAAV9-amiR-ctrl, OVX+rAAV9-amiR-ctrl, OVX+rAAV9-amiR-shn3, sham+rAAV9. DSS-Nter-amiR-ctrl, OVX+rAAV9. DSS-Nter-amiR-ctrl, OVX+TAAV9. DSS-Nter-amiR-shn3. Seven weeks after the injection, mice were subcutaneously injected with calcein and alizarin-3-methyliminodiacetic acid at six day-intervals for dynamic histomorphometric analysis. Non-labeled mice were used to monitor EGFP expression using the IVIS-100 optical imaging or frozen-sections.

### Statistical Methods

All data were presented as the mean+s.e.m. Sample sizes were calculated on the assumption that a 30% difference in the parameters measured would be considered biologically significant with an estimate of sigma of 10-20% of the expected mean. Alpha and Beta were set to the standard values of.05 and 0.8, respectively. No animals or samples were excluded from analysis, and animals were randomized to treatment versus control groups, where applicable. For relevant data analysis, where relevant, the Shapiro-Wilk normality test was first performed for checking normal distributions of the groups. If normality tests passed, two-tailed, unpaired Student's/-test and if normality tests failed, and Mann-Whitney tests were used for the comparisons between two groups. For the comparisons of three or four groups, one-way ANOVA was used if normality tests passed, followed by Tukey's multiple comparison test for all pairs of groups. If normality tests failed, Kruskal-Wallis test was performed and was followed by Dunn's multiple comparison test. The GraphPad PRISM software (v6.0a, La Jolla, CA) was used for statistical analysis. P<0.05 was considered statistically significant. *, P<0.05; **, P<0.01; ***, P<0.001; and ****, P<0.0001.

SEQUENCES
>SEQ ID NO: 1; amiR-33-mSHN3i-1:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGTACAAACTACTTGAGAGCAGGTGTTCTGGCAATACCTGCCTG
CTCTGTAATAGTTTGTACACGGAGGCCTGCCCTGACTGCCCACGGTGCCGTGGCC
AAAGAGGATCTAAGGGCACCGCTGAGGGCCTACCTAACCATCGTGGGGAATAAGG
ACAGTGTCACCCCTGCAGgggatccggtggtggtgcaaatca
>SEQ ID NO: 2; amiR-33-mSHN3i-2:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGACTACAGGTACTCACAAGCTCTGTTCTGGCAATACCTGGAGCT
TGTCTGCACCTGTAGTCACGGAGGCCTGCCCTGACTGCCCACGGTGCCGTGGCCA
AAGAGGATCTAAGGGCACCGCTGAGGGCCTACCTAACCATCGTGGGGAATAAGGA
CAGTGTCACCCCTGCAGgggatccggtggtggtgcaaatca
>SEQ ID NO: 3; amiR-33-hSHN3i-1:
GtcttttatttcaggtcccagatcttAGGGCTCTGCGTTTGCTCCAGGTAGTCCGCTGCTCCCTTGGG
CCTGGGCCCACTGACAGCCCTGGTGCCTCTGGCCGGCTGCACACCTCCTGGCGGGC
AGCTGTGtttccatggtaagttcaaggcTGTTCTGGCAATACCTGGCCTTGAAGATGCCATGGAA
ACACGGAGGCCTGCCCTGACTGCCCACGGTGCCGTGGCCAAAGAGGATCTAAGGGC
ACCGCTGAGGGCCTACCTAACCATCGTGGGGAATAAGGACAGTGTCACCCccctgcagg
ggatccggtggtggtgcaaat
>SEQ ID NO: 4; amiR-33-mCTSKi-1:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGTTTCATCATAGTACACACCTCTGTTCTGGCAATACCTGGAGGT
GTGATCCATGATGAAACACGGAGGCCTGCCCTGACTGCCCACGGTGCCGTGGCCA
AAGAGGATCTAAGGGCACCGCTGAGGGCCTACCTAACCATCGTGGGGAATAAGGA
CAGTGTCACCCCTGCAGgggatccggtggtggtgcaaatca
>SEQ ID NO: 5; amiR-33-mCTSKi-2:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGTTACTGTAGGATCGAGAGGGATGTTCTGGCAATACCTGTCCC
TCTCCTTACTACAGTAACACGGAGGCCTGCCCTGACTGCCCACGGTGCCGTGGCC
AAAGAGGATCTAAGGGCACCGCTGAGGGCCTACCTAACCATCGTGGGGAATAAGG
>SEQ ID NO: 6; amiR-33-hCTSK-1:
GtcttttatttcaggtcccagatcttAGGGCTCTGCGTTTGCTCCAGGTAGTCCGCTGCTCCCTTGGG
CCTGGGCCCACTGACAGCCCTGGTGCCTCTGGCCGGCTGCACACCTCCTGGCGGGC
AGCTGTGattatcgctattgcagctttcTGTTCTGGCAATACCTGGAAAGCTGGTACAGCGATAAT
CACGGAGGCCTGCCCTGACTGCCCACGGTGCCGTGGCCAAAGAGGATCTAAGGGCA
CCGCTGAGGGCCTACCTAACCATCGTGGGGAATAAGGACAGTGTCACCC
ccctgcaggggatccggtggtggtgcaaat
>SEQ ID NO: 7; amiR-33-hCTSK-2:
GtcttttatttcaggtcccagatcttAGGGCTCTGCGTTTGCTCCAGGTAGTCCGCTGCTCCCTTGGG
CCTGGGCCCACTGACAGCCCTGGTGCCTCTGGCCGGCTGCACACCTCCTGGCGGGC
AGCTGTGtcagattatcgctattgcagcTGTTCTGGCAATACCTGGCTGCAATTCCAATAATCTGA
CACGGAGGCCTGCCCTGACTGCCCACGGTGCCGTGGCCAAAGAGGATCTAAGGGCA
CCGCTGAGGGCCTACCTAACCATCGTGGGGAATAAGGACAGTGTCACCC
ccctgcaggggatccggtggtggtgcaaat
>SEQ ID NO: 8; amiR-33-mSOSTi-1:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGAcaagtaggcagatgaggcacTGTTCTGGCAATACCTGGTGCCTCAAG
TACCTACTTGTCACGGAGGCCTGCCCTGACTGCCCACGGTGCCGTGGCCAAAGAG
GATCTAAGGGCACCGCTGAGGGCCTACCTAACCATCGTGGGGAATAAGGACAGTG
TCACCCCTGCAGgggatccggtggtggtgcaaatca
>SEQ ID NO: 9; amiR-33-mSOSTi-2:
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGtgacctctgtggcatcattccTGTTCTGGCAATACCTGGGAATGATCGCG
CAGAGGTCACACGGAGGCCTGCCCTGACTGCCCACGGTGCCGTGGCCAAAGAGG
ATCTAAGGGCACCGCTGAGGGCCTACCTAACCATCGTGGGGAATAAGGACAGTGT
CACCCCTGCAGgggatccggtggtggtgcaaatca
>SEQ ID NO: 10; amiR-33-hSosT-1:
GtcttttatttcaggtcccagatcttAGGGCTCTGCGTTTGCTCCAGGTAGTCCGCTGCTCCCTTGGG
CCTGGGCCCACTGACAGCCCTGGTGCCTCTGGCCGGCTGCACACCTCCTGGCGGGC
AGCTGTGatggtcttgttgttctccagcTGTTCTGGCAATACCTGGCTGGAGATGAGCAAGACCA
TCACGGAGGCCTGCCCTGACTGCCCACGGTGCCGTGGCCAAAGAGGATCTAAGGGC
ACCGCTGAGGGCCTACCTAACCATCGTGGGGAATAAGGACAGTGTCACCC
ccctgcaggggatccggtggtggtgcaaat
>SEQ ID NO: 11; amiR-33-hSosT-2:
GtcttttatttcaggtcccagatcttAGGGCTCTGCGTTTGCTCCAGGTAGTCCGCTGCTCCCTTGGG
CCTGGGCCCACTGACAGCCCTGGTGCCTCTGGCCGGCTGCACACCTCCTGGCGGGC
AGCTGTGACGtCtttGGtCtCAAAGGGGTGTTCTGGCAATACCTGCCCCTTTGTCATCAA
AGACGTCACGGAGGCCTGCCCTGACTGCCCACGGTGCCGTGGCCAAAGAGGATCTA
AGGGCACCGCTGAGGGCCTACCTAACCATCGTGGGGAATAAGGACAGTGTCACCC
ccctgcaggggatccggtggtggtgcaaat
>SEQ ID NO: 12; Human PTH (1-84 aa):
ggaattgtacccgcggccgatccAccggtGCCACCATGATACCTGCAAAAGACATGGCTAAAGTTA
TGATTGTCATGTTGGCAATTTGTTTTCTTACAAAATCGGATGGGAAATCTGTTAAGA
AGAGATCTGTGAGTGAAATACAGCTTATGCATAACCTGGGAAAACATCTGAACTCG
ATGGAGAGAGTAGAATGGCTGCGTAAGAAGCTGCAGGATGTGCACAATTTTGTTGC
CCTTGGAGCTCCTCTAGCTCCCAGAGATGCTGGTTCCCAGAGGCCCCGAAAAAAGG
AAGACAATGTCTTGGTTGAGAGCCATGAAAAAAGTCTTGGAGAGGCAGACAAAGCT
GATGTGAATGTATTAACTAAAGCTAAATCCCAGTGAagcttatcgataccgtcgactagagctc
>SEQ ID NO: 13; Human PTHrP (1-140 aa):
ggaattgtacccgcggccgatccAccggtGCCACCATGCAGCGGAGACTGGTTCAGCAGTGGAGCG
TCGCGGTGTTCCTGCTGAGCTACGCGGTGCCCTCCTGCGGGCGCTCGGTGGAGGGTC
TCAGCCGCCGCCTCAAAAGAGCTGTGTCTGAACATCAGCTCCTCCATGACAAGGGG
AAGTCCATCCAAGATTTACGGCGACGATTCTTCCTTCACCATCTGATCGCAGAAATC
CACACAGCTGAAATCAGAGCTACCTCGGAGGTGTCCCCTAACTCCAAGCCCTCTCCC
AACACAAAGAACCACCCCGTCCGATTTGGGTCTGATGATGAGGGCAGATACCTAAC
TCAGGAAACTAACAAGGTGGAGACGTACAAAGAGCAGCCGCTCAAGACACCTGGG
AAGAAAAAGAAAGGCAAGCCCGGGAAACGCAAGGAGCAGGAAAAGAAAAAACGG
CGAACTCGCTCTGCCTGGTTAGACTCTGGAGTGACTGGGAGTGGGCTAGAAGGGGA
CCACCTGTCTGACACCTCCACAACGTCGCTGGAGCTCGATTCACGGTAAagcttatcgatac
cgtcgactagagctc
>SEQ ID NO: 14; Mouse DJ-1 (1-190 aa):
ggaattgtacccgcggccgatccaccggtcGCCACCATGGGATGGAGCTGTATTATCCTGTTTCT
CGTCGCTACTGCCACCGGAGCTCATTCCGCTTCCAAAAGAGCTCTGGTCATCCTGG
CCAAAGGAGCAGAGGAGATGGAGACAGTGATTCCTGTGGATGTCATGCGGCGAGC
CGGGATCAAAGTCACTGTTGCAGGCTTGGCTGGGAAGGACCCCGTGCAGTGTAGC
CGTGATGTAATGATTTGTCCAGATACCAGTCTGGAAGATGCAAAAACGCAGGGACC
ATACGATGTGGTGGTTCTTCCAGGAGGAAATCTGGGTGCACAGAATTTATCTGAGT
CGCCTATGGTGAAGGAGATCCTCAAGGAGCAGGAGAGCAGGAAGGGCCTCATAGC
TGCCATCTGTGCAGGTCCTACGGCTCTGTTGGCTCACGAAGTAGGTTTTGGATGCA
AGGTCACAACACACCCACTGGCTAAGGACAAAATGATGAATGGCAGTCACTACAGC
TACTCAGAGAGCCGCGTGGAGAAGGACGGCCTGATCCTCACCAGCCGCGGGCCG
GGGACCAGCTTTGAGTTTGCACTAGCCATTGTGGAGGCACTCGTGGGGAAAGACA
TGGCCAACCAAGTGAAGGCACCGCTTGTTCTCAAAGACTAGTAAaagcttatcgataccgtc
gactagagctcgctg
>SEQ ID NO: 15; Human DJ-1 (1-190 aa):
ggaattgtacccgcggccgatccaccggtc GCCACC ATG GGA TGG AGC TGT ATT
ATC CTG TTT CTC GTC GCT ACT GCC ACC GGA GCT CAT
TCCGCTTCCAAAAGAGCTCTGGTCATCCTGGCTAAAGGAGCAGAGGAAATGGAGAC
GGTCATCCCTGTAGATGTCATGAGGCGAGCTGGGATTAAGGTCACCGTTGCAGGCC
TGGCTGGAAAAGACCCAGTACAGTGTAGCCGTGATGTGGTCATTTGTCCTGATGCC
AGCCTTGAAGATGCAAAAAAAGAGGGACCATATGATGTGGTGGTTCTACCAGGAGG
TAATCTGGGCGCACAGAATTTATCTGAGTCTGCTGCTGTGAAGGAGATACTGAAGG
AGCAGGAAAACCGGAAGGGCCTGATAGCCGCCATCTGTGCAGGTCCTACTGCTCTG
TTGGCTCATGAAATAGGTTTTGGAAGTAAAGTTACAACACACCCTCTTGCTAAAGAC
AAAATGATGAATGGAGGTCATTACACCTACTCTGAGAATCGTGTGGAAAAAGACGG
CCTGATTCTTACAAGCCGGGGGCCTGGGACCAGCTTCGAGTTTGCGCTTGCAATTGT
TGAAGCCCTGAATGGCAAGGAGGTGGCGGCTCAAGTGAAGGCTCCACTTGTTCTTA
AAGACTAGTAAaagcttatcgataccgtcgactagagctcgctg
>SEQ ID NO: 16; (AspSerSer)₆ Bone-targeting Peptide
DSSDSSDSSDSSDSSDSS
>SEQ ID NO: 17; HABP-19, Bone-targeting Peptide
CγEPRRγEVAγELγEPRRγEVAγEL,
γ(Gla residue): γ-carboxylated glutamic acid (Glu) is derived
from Glu by vitamin K-dependent γ-carboxylation.
>SEQ ID NO 18: AAV-CAPSID 1;
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEGAKTAPGKKRPVEQSPQEPDSSSGIGKTGQQPAKKR
LNFGQTGDSESVPDPQPLGEPPATPAAVGPTTMASGGGAPMADNNEGADGVGNASGN
WHCDSTWLGDRVITTSTRTWALPTYNNHLYKQISSASTGASNDNHYFGYSTPWGYFDF
NRFHCHFSPRDWQRLINNNWGFRPKRLNFKLFNIQVKEVTTNDGVTTIANNLTSTVQVF
SDSEYQLPYVLGSAHQGCLPPFPADVFMIPQYGYLTLNNGSQAVGRSSFYCLEYFPSQM
LRTGNNFTFSYTFEEVPFHSSYAHSQSLDRLMNPLIDQYLYYLNRTQNQSGSAQNKDLL
FSRGSPAGMSVQPKNWLPGPCYRQQRVSKTKTDNNNSNFTWTGASKYNLNGRESIINP
GTAMASHKDDEDKFFPMSGVMIFGKESAGASNTALDNVMITDEEEIKATNPVATERFG
TVAVNFQSSSTDPATGDVHAMGALPGMVWQDRDVYLQGPIWAKIPHTDGHFHPSPLM
GGFGLKNPPPQILIKNTPVPANPPAEFSATKFASFITQYSTGQVSVEIEWELQKENSKRW
NPEVQYTSNYAKSANVDFTVDNNGLYTEPRPIGTRYLTRPL
>SEQ ID NO 19: AAV-CAPSID 2;
MAADGYLPDWLEDTLSEGIRQWWKLKPGPPPPKPAERHKDDSRGLVLPGYKYLGPFN
GLDKGEPVNEADAAALEHDKAYDRQLDSGDNPYLKYNHADAEFQERLKEDTSFGGNL
GRAVFQAKKRVLEPLGLVEEPVKTAPGKKRPVEHSPVEPDSSSGTGKAGQQPARKRLN
FGQTGDADSVPDPQPLGQPPAAPSGLGTNTMATGSGAPMADNNEGADGVGNSSGNWH
CDSTWMGDRVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLTSTVQVFTDS
EYQLPYVLGSAHQGCLPPFPADVFMVPQYGYLTLNNGSQAVGRSSFYCLEYFPSQMLR
TGNNFTFSYTFEDVPFHSSYAHSQSLDRLMNPLIDQYLYYLSRTNTPSGTTTQSRLQFSQ
AGASDIRDQSRNWLPGPCYRQQRVSKTSADNNNSEYSWTGATKYHLNGRDSLVNPGP
AMASHKDDEEKFFPQSGVLIFGKQGSEKTNVDIEKVMITDEEEIRTTNPVATEQYGSVST
NLQRGNRQAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWELQKENSKRWNPEI
QYTSNYNKSVNVDFTVDTNGVYSEPRPIGTRYLTRNL
>SEQ ID NO 20: AAV-CAPSID 3B;
MAADGYLPDWLEDNLSEGIREWWALKPGVPQPKANQQHQDNRRGLVLPGYKYLGPG
NGLDKGEPVNEADAAALEHDKAYDQQLKAGDNPYLKYNHADAEFQERLQEDTSFGG
NLGRAVFQAKKRILEPLGLVEEAAKTAPGKKRPVDQSPQEPDSSSGVGKSGKQPARKR
LNFGQTGDSESVPDPQPLGEPPAAPTSLGSNTMASGGGAPMADNNEGADGVGNSSGN
WHCDSQWLGDRVITTSTRTWALPTYNNHLYKQISSQSGASNDNHYFGYSTPWGYFDF
NRFHCHFSPRDWQRLINNNWGFRPKKLSFKLFNIQVKEVTQNDGTTTIANNLTSTVQVF
TDSEYQLPYVLGSAHQGCLPPFPADVFMVPQYGYLTLNNGSQAVGRSSFYCLEYFPSQ
MLRTGNNFQFSYTFEDVPFHSSYAHSQSLDRLMNPLIDQYLYYLNRTQGTTSGTTNQSR
LLFSQAGPQSMSLQARNWLPGPCYRQQRLSKTANDNNNSNFPWTAASKYHLNGRDSL
VNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSP
LMGGFGLKHPPPQIMIKNTPVPANPPTTFSPAKFASFITQYSTGQVSVEIEWELQKENSK
RWNPEIQYTSNYNKSVNVDFTVDTNGVYSEPRPIGTRYLTRNL
>SEQ ID NO 21: AAV-CAPSID 4:
MTDGYLPDWLEDNLSEGVREWWALQPGAPKPKANQQHQDNARGLVLPGYKYLGPGN
GLDKGEPVNAADAAALEHDKAYDQQLKAGDNPYLKYNHADAEFQQRLQGDTSFGGN
LGRAVFQAKKRVLEPLGLVEQAGETAPGKKRPLIESPQQPDSSTGIGKKGKQPAKKKLV
FEDETGAGDGPPEGSTSGAMSDDSEMRAAAGGAAVEGGQGADGVGNASGDWHCDST
WSEGHVTTTSTRTWVLPTYNNHLYKRLGESLQSNTYNGFSTPWGYFDFNRFHCHFSPR
DWQRLINNNWGMRPKAMRVKIFNIQVKEVTTSNGETTVANNLTSTVQIFADSSYELPY
VMDAGQEGSLPPFPNDVFMVPQYGYCGLVTGNTSQQQTDRNAFYCLEYFPSQMLRTG
NNFEITYSFEKVPFHSMYAHSQSLDRLMNPLIDQYLWGLQSTTTGTTLNAGTATTNFTK
LRPTNFSNFKKNWLPGPSIKQQGFSKTANQNYKIPATGSDSLIKYETHSTLDGRWSALTP
GPPMATAGPADSKFSNSQLIFAGPKQNGNTATVPGTLIFTSEEELAATNATDTDMWGNL
PGGDQSNSNLPTVDRLTALGAVPGMVWQNRDIYYQGPIWAKIPHTDGHFHPSPLIGGFG
LKHPPPQIFIKNTPVPANPATTFSSTPVNSFITQYSTGQVSVQIDWEIQKERSKRWNPEVQ
FTSNYGQQNSLLWAPDAAGKYTEPRAIGTRYLTHHL
>SEQ ID NO 22: AAV-CAPSID 5:
MSFVDHPPDWLEEVGEGLREFLGLEAGPPKPKPNQQHQDQARGLVLPGYNYLGPGNG
LDRGEPVNRADEVAREHDISYNEQLEAGDNPYLKYNHADAEFQEKLADDTSFGGNLG
KAVFQAKKRVLEPFGLVEEGAKTAPTGKRIDDHFPKRKKARTEEDSKPSTSSDAEAGPS
GSQQLQIPAQPASSLGADTMSAGGGGPLGDNNQGADGVGNASGDWHCDSTWMGDRV
VTKSTRTWVLPSYNNHQYREIKSGSVDGSNANAYFGYSTPWGYFDFNRFHSHWSPRD
WQRLINNYWGFRPRSLRVKIFNIQVKEVTVQDSTTTIANNLTSTVQVFTDDDYQLPYVV
GNGTEGCLPAFPPQVFTLPQYGYATLNRDNTENPTERSSFFCLEYFPSKMLRTGNNFEFT
YNFEEVPFHSSFAPSQNLFKLANPLVDQYLYRFVSTNNTGGVQFNKNLAGRYANTYKN
WFPGPMGRTQGWNLGSGVNRASVSAFATTNRMELEGASYQVPPQPNGMTNNLQGSN
TYALENTMIFNSQPANPGTTATYLEGNMLITSESETQPVNRVAYNVGGQMATNNQSST
TAPATGTYNLQEIVPGSVWMERDVYLQGPIWAKIPETGAHFHPSPAMGGFGLKHPPPM
MLIKNTPVPGNITSFSDVPVSSFITQYSTGQVTVEMEWELKKENSKRWNPEIQYTNNYN
DPQFVDFAPDSTGEYRTTRPIGTRYLTRPL
>SEQ ID NO 23: AAV-CAPSID 6:
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPFGLVEEGAKTAPGKKRPVEQSPQEPDSSSGIGKTGQQPAKKRL
NFGQTGDSESVPDPQPLGEPPATPAAVGPTTMASGGGAPMADNNEGADGVGNASGNW
HCDSTWLGDRVITTSTRTWALPTYNNHLYKQISSASTGASNDNHYFGYSTPWGYFDFN
RFHCHFSPRDWQRLINNNWGFRPKRLNFKLFNIQVKEVTTNDGVTTIANNLTSTVQVFS
DSEYQLPYVLGSAHQGCLPPFPADVFMIPQYGYLTLNNGSQAVGRSSFYCLEYFPSQML
RTGNNFTFSYTFEDVPFHSSYAHSQSLDRLMNPLIDQYLYYLNRTQNQSGSAQNKDLLF
SRGSPAGMSVQPKNWLPGPCYRQQRVSKTKTDNNNSNFTWTGASKYNLNGRESIINPG
TAMASHKDDKDKFFPMSGVMIFGKESAGASNTALDNVMITDEEEIKATNPVATERFGT
VAVNLQSSSTDPATGDVHVMGALPGMVWQDRDVYLQGPIWAKIPHTDGHFHPSPLMG
GFGLKHPPPQILIKNTPVPANPPAEFSATKFASFITQYSTGQVSVEIEWELQKENSKRWNP
EVQYTSNYAKSANVDFTVDNNGLYTEPRPIGTRYLTRPL
>SEQ ID NO 24: AAV-CAPSID 6.2;
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEGAKTAPGKKRPVEQSPQEPDSSSGIGKTGQQPAKKR
LNFGQTGDSESVPDPQPLGEPPATPAAVGPTTMASGGGAPMADNNEGADGVGNASGN
WHCDSTWLGDRVITTSTRTWALPTYNNHLYKQISSASTGASNDNHYFGYSTPWGYFDF
NRFHCHFSPRDWQRLINNNWGFRPKRLNFKLFNIQVKEVTTNDGVTTIANNLTSTVQVF
SDSEYQLPYVLGSAHQGCLPPFPADVFMIPQYGYLTLNNGSQAVGRSSFYCLEYFPSQM
LRTGNNFTFSYTFEDVPFHSSYAHSQSLDRLMNPLIDQYLYYLNRTQNQSGSAQNKDLL
FSRGSPAGMSVQPKNWLPGPCYRQQRVSKTKTDNNNSNFTWTGASKYNLNGRESIINP
GTAMASHKDDKDKFFPMSGVMIFGKESAGASNTALDNVMITDEEEIKATNPVATERFG
TVAVNLQSSSTDPATGDVHVMGALPGMVWQDRDVYLQGPIWAKIPHTDGHFHPSPLM
GGFGLKHPPPQILIKNTPVPANPPAEFSATKFASFITQYSTGQVSVEIEWELQKENSKRW
NPEVQYTSNYAKSANVDFTVDNNGLYTEPRPIGTRYLTRPL
>SEQ ID NO 25: AAV-CAPSID 7:
MAADGYLPDWLEDNLSEGIREWWDLKPGAPKPKANQQKQDNGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEGAKTAPAKKRPVEPSPQRSPDSSTGIGKKGQQPARKR
LNFGQTGDSESVPDPQPLGEPPAAPSSVGSGTVAAGGGAPMADNNEGADGVGNASGN
WHCDSTWLGDRVITTSTRTWALPTYNNHLYKQISSETAGSTNDNTYFGYSTPWGYFDF
NRFHCHFSPRDWQRLINNNWGFRPKKLRFKLFNIQVKEVTTNDGVTTIANNLTSTIQVF
SDSEYQLPYVLGSAHQGCLPPFPADVFMIPQYGYLTLNNGSQSVGRSSFYCLEYFPSQM
LRTGNNFEFSYSFEDVPFHSSYAHSQSLDRLMNPLIDQYLYYLARTQSNPGGTAGNREL
QFYQGGPSTMAEQAKNWLPGPCFRQQRVSKTLDQNNNSNFAWTGATKYHLNGRNSL
VNPGVAMATHKDDEDRFFPSSGVLIFGKTGATNKTTLENVLMTNEEEIRPTNPVATEEY
GIVSSNLQAANTAAQTQVVNNQGALPGMVWQNRDVYLQGPIWAKIPHTDGNFHPSPL
MGGFGLKHPPPQILIKNTPVPANPPEVFTPAKFASFITQYSTGQVSVEIEWELQKENSKR
WNPEIQYTSNFEKQTGVDFAVDSQGVYSEPRPIGTRYLTRNL
>SEQ ID NO 26: AAV-CAPSID 8:
MAADGYLPDWLEDNLSEGIREWWALKPGAPKPKANQQKQDDGRGLVLPGYKYLGPF
NGLDKGEPVNAADAAALEHDKAYDQQLQAGDNPYLRYNHADAEFQERLQEDTSFGG
NLGRAVFQAKKRVLEPLGLVEEGAKTAPGKKRPVEPSPQRSPDSSTGIGKKGQQPARKR
LNFGQTGDSESVPDPQPLGEPPAAPSGVGPNTMAAGGGAPMADNNEGADGVGSSSGN
WHCDSTWLGDRVITTSTRTWALPTYNNHLYKQISNGTSGGATNDNTYFGYSTPWGYF
DFNRFHCHFSPRDWQRLINNNWGFRPKRLSFKLFNIQVKEVTQNEGTKTIANNLTSTIQ
VFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTQTTGGTANTQT
LGFSQGGPNTMANQAKNWLPGPCYRQQRVSTTTGQNNNSNFAWTAGTKYHLNGRNS
LANPGIAMATHKDDEERFFPSNGILIFGKQNAARDNADYSDVMLTSEEEIKTTNPVATE
EYGIVADNLQQQNTAPQIGTVNSQGALPGMVWQNRDVYLQGPIWAKIPHTDGNFHPSP
LMGGFGLKHPPPQILIKNTPVPADPPTTFNQSKLNSFITQYSTGQVSVEIEWELQKENSKR
WNPEIQYTSNYYKSTSVDFAVNTEGVYSEPRPIGTRYLTRNL
>SEQ ID NO 27: AAV-CAPSID 9:
MAADGYLPDWLEDNLSEGIREWWALKPGAPQPKANQQHQDNARGLVLPGYKYLGPG
NGLDKGEPVNAADAAALEHDKAYDQQLKAGDNPYLKYNHADAEFQERLKEDTSFGG
NLGRAVFQAKKRLLEPLGLVEEAAKTAPGKKRPVEQSPQEPDSSAGIGKSGAQPAKKR
LNFGQTGDTESVPDPQPIGEPPAAPSGVGSLTMASGGGAPVADNNEGADGVGSSSGNW
HCDSQWLGDRVITTSTRTWALPTYNNHLYKQISNSTSGGSSNDNAYFGYSTPWGYFDF
NRFHCHFSPRDWQRLINNNWGFRPKRLNFKLFNIQVKEVTDNNGVKTIANNLTSTVQV
FTDSDYQLPYVLGSAHEGCLPPFPADVFMIPQYGYLTLNDGSQAVGRSSFYCLEYFPSQ
MLRTGNNFQFSYEFENVPFHSSYAHSQSLDRLMNPLIDQYLYYLSKTINGSGQNQQTLK
FSVAGPSNMAVQGRNYIPGPSYRQQRVSTTVTQNNNSEFAWPGASSWALNGRNSLMN
PGPAMASHKEGEDRFFPLSGSLIFGKQGTGRDNVDADKVMITNEEEIKTTNPVATESYG
QVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>SEQ ID NO 28: AAV-CAPSID rh8:
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEGAKTAPGKKRPVEQSPQEPDSSSGIGKTGQQPAKKR
LNFGQTGDSESVPDPQPLGEPPAAPSGLGPNTMASGGGAPMADNNEGADGVGNSSGN
WHCDSTWLGDRVITTSTRTWALPTYNNHLYKQISNGTSGGSTNDNTYFGYSTPWGYFD
FNRFHCHFSPRDWQRLINNNWGFRPKRLNFKLFNIQVKEVTTNEGTKTIANNLTSTVQV
FTDSEYQLPYVLGSAHQGCLPPFPADVFMVPQYGYLTLNNGSQALGRSSFYCLEYFPSQ
MLRTGNNFQFSYTFEDVPFHSSYAHSQSLDRLMNPLIDQYLYYLVRTQTTGTGGTQTLA
FSQAGPSSMANQARNWVPGPCYRQQRVSTTTNQNNNSNFAWTGAAKFKLNGRDSLM
NPGVAMASHKDDDDRFFPSSGVLIFGKQGAGNDGVDYSQVLITDEEEIKATNPVATEEY
GAVAINNQAANTQAQTGLVHNQGVIPGMVWQNRDVYLQGPIWAKIPHTDGNFHPSPL
MGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL
>SEQ ID NO 29: AAV-CAPSID rh10:
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEGAKTAPGKKRPVEPSPQRSPDSSTGIGKKGQQPAKK
RLNFGQTGDSESVPDPQPIGEPPAGPSGLGSGTMAAGGGAPMADNNEGADGVGSSSGN
WHCDSTWLGDRVITTSTRTWALPTYNNHLYKQISNGTSGGSTNDNTYFGYSTPWGYFD
FNRFHCHFSPRDWQRLINNNWGFRPKRLNFKLFNIQVKEVTQNEGTKTIANNLTSTIQV
FTDSEYQLPYVLGSAHQGCLPPFPADVFMIPQYGYLTLNNGSQAVGRSSFYCLEYFPSQ
MLRTGNNFEFSYQFEDVPFHSSYAHSQSLDRLMNPLIDQYLYYLSRTQSTGGTAGTQQL
LFSQAGPNNMSAQAKNWLPGPCYRQQRVSTTLSQNNNSNFAWTGATKYHLNGRDSLV
NPGVAMATHKDDEERFFPSSGVLMFGKQGAGKDNVDYSSVMLTSEEEIKTTNPVATEQ
YGVVADNLQQQNAAPIVGAVNSQGALPGMVWQNRDVYLQGPIWAKIPHTDGNFHPSP
LMGGFGLKHPPPQILIKNTPVPADPPTTFSQAKLASFITQYSTGQVSVEIEWELQKENSKR
WNPEIQYTSNYYKSTNVDFAVNTDGTYSEPRPIGTRYLTRNL
>SEQ ID NO 30: AAV-CAPSID rh39:
MAADGYLPDWLEDNLSEGIREWWALKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEAAKTAPGKKRPVEPSPQRSPDSSTGIGKKGQQPAKK
RLNFGQTGDSESVPDPQPIGEPPAGPSGLGSGTMAAGGGAPMADNNEGADGVGSSSGN
WHCDSTWLGDRVITTSTRTWALPTYNNHLYKQISNGTSGGSTNDNTYFGYSTPWGYFD
FNRFHCHFSPRDWQRLINNNWGFRPKRLSFKLFNIQVKEVTQNEGTKTIANNLTSTIQVF
TDSEYQLPYVLGSAHQGCLPPFPADVFMIPQYGYLTLNNGSQAVGRSSFYCLEYFPSQM
LRTGNNFEFSYTFEDVPFHSSYAHSQSLDRLMNPLIDQYLYYLSRTQSTGGTQGTQQLL
FSQAGPANMSAQAKNWLPGPCYRQQRVSTTLSQNNNSNFAWTGATKYHLNGRDSLV
NPGVAMATHKDDEERFFPSSGVLMFGKQGAGRDNVDYSSVMLTSEEEIKTTNPVATEQ
YGVVADNLQQTNTGPIVGNVNSQGALPGMVWQNRDVYLQGPIWAKIPHTDGNFHPSP
LMGGFGLKHPPPQILIKNTPVPADPPTTFSQAKLASFITQYSTGQVSVEIEWELQKENSKR
WNPEIQYTSNYYKSTNVDFAVNTEGTYSEPRPIGTRYLTRNL
>SEQ ID NO 31: AAV-CAPSID rh43:
MAADGYLPDWLEDNLSEGIREWWDLKPGAPKPKANQQKQDDGRGLVLPGYKYLGPF
NGLDKGEPVNAADAAALEHDKAYDQQLEAGDNPYLRYNHADAEFQERLQEDTSFGG
NLGRAVFQAKKRVLEPLGLVEEGAKTAPGKKRPVEQSPQEPDSSSGIGKKGQQPARKR
LNFGQTGDSESVPDPQPLGEPPAAPSGVGPNTMAAGGGAPMADNNEGADGVGSSSGN
WHCDSTWLGDRVITTSTRTWALPTYNNHLYKQISNGTSGGATNDNTYFGYSTPWGYF
DFNRFHCHFSPRDWQRLINNNWGFRPKRLSFKLFNIQVKEVTQNEGTKTIANNLTSTIQ
VFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTQTTGGTANTQT
LGFSQGGPNTMANQAKNWLPGPCYRQQRVSTTTGQNNNSNFAWTAGTKYHLNGRNS
LANPGIAMATHKDDEERFFPSNGILIFGKQNAARDNADYSDVMLTSEEEIKTTNPVATE
EYGIVADNLQQQNTAPQIGTVNSQGALPGMVWQNRDVYLQGPIWAKIPHTDGNFHPSP
LMGGFGLKHPPPQILIKNTPVPADPPTTFNQSKLNSFITQYSTGQVSVEIEWELQKENSKR
WNPEIQYTSNYYKSTSVDFAVNTEGVYSEPRPIGTRYLTRNL
>SEQ ID NO 32: AAV-CAPSID 2/2-66:
MAADGYLPDWLEDTLSEGIRQWWKLKPGPPPPKPAERHQDDSRGLVLPGYKYLGPFN
GLDKGEPVNEADAAALEHDKAYDRQLDSGDNPYLKYNHADAEFQERLKEDTSFGGNL
GRAVFQAKKRVLEPLGLVEEPVKTAPGKKRPVEHSPAEPDSSSGTGKAGQQPARKRLN
FGQTGDADSVPDPQPLGQPPAAPSGLGTNTMATGSGAPMADNNEGADGVGNSSGNWH
CDSTWMGDRVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLTSTVQVFTDS
EYQLPYVLGSAHQGCLPPFPADVFMVPQYGYLTLNNGSQAVGRSSFYCLEYFPSQMLR
TGNNFTFSYTFEDVPFHSSYAHSQSLDRLMNPLIDQYLYYLSKTNAPSGTTTMSRLQFSQ
AGASDIRDQSRNWLPGPCYRQQRVSKTAADNNNSDYSWTGATKYHLNGRDSLVNPGP
AMASHKDDEEKYFPQSGVLIFGKQDSGKTNVDIEKVMITDEEEIRTTNPVATEQYGSVS
TNLQSGNTQAATTDVNTQGVLPGMVWQDRDVYLQGPIWAKIPHTDGHFHPSPLMGGF
GLKHPPPQILIKNTPVPANPSTTFSAAKFASFITQYSTGQVSVEIEWELQKENSKRWNPEI
QYTSNYNKSVNVDFTVDTNGVYSEPRPIGTRYLTRNL
>SEQ ID NO 33: AAV-CAPSID 2/2-84:
MAADGYLPDWLEDTLSEGIRQWWKLKPGPPPPKPAERHQDDSRGLVLPGYKYLGPFN
GLDKGEPVNEADAAALEHDKAYDRQLDSGDNPYLKYNHADAEFQERLKEDTSFGGNL
GRAVFQAKKRVLEPLGLVEEPVKTAPGKKRPVEHSPAEPDSSSGTGKAGQQPARKRLN
FGQTGDADSVPDPQPLGQPPAAPSGLGTNTMATGSGAPMADNNEGADGVGNSSGNWH
CDSTWMGDRVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLTSTVQVFTDS
EYQLPYVLGSAHQGCLPPFPADVFMVPQYGYLTLNNGSQAVGRSSFYCLEYFPSQMLR
TGNNFTFSYTFEDVPFHSSYAHSQSLDRLMNPLIDQYLYYLSKTNAPSGTTTMSRLQFSQ
AGASDIRDQSRNWLPGPCYRQQRVSKTAADNNNSDYSWTGATKYHLNGRDSLVNPGP
AMASHKDDEEKYFPQSGVLIFGKQDSGKTNVDIEKVMITDEEEIRTTNPVATEQYGSVS
TNLQSGNTQAATTDVNTQGVLPGMVWQDRDVYLQGPIWAKIPHTDGHFHPSPLMGGF
GLKHPPPQILIKNTPVPANPSTTFSAAKLASFITQYSTGQVSVEIEWELQKENSKRWNPEI
QYTSNYNKSVNVDFTVDTNGVYSEPRPIGTRYLTRNL
>SEQ ID NO 34: AAV-CAPSID 2/2-125:
MAADGYLPDWLEDTLSEGIRQWWKLKPGPPPPKPAERHKDDSRGLVLPGYKYLGPFN
GLDKGEPVNEADAAALEHDKAYDRQLDSGDNPYLKYNHADAEFQERLKEDTSFGGNL
ARAVFQAKKRVLEPLGLVEEPVKTAPGKKRPVEHSPAEPDSSSGTGKSGQQPARKRLNF
GQTGDADSVPDPQPLGQPPAAPSGLGTNTMASGSGAPMADNNEGADGVGNSSGNWH
CDSTWMGDRVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLTSTVQVFTDS
EYQLPYVLGSAHQGCLPPFPADVFMVPQYGYLTLNNGSQTVGRSSFYCLEYFPSQMLR
TGNNFTFSYTFEDVPFHSSYAHSQSLDRLMNPLIDQYLYYLSRTNTPSGTTTQSRLRFSQ
AGASDIRDQSRNWLPGPCYRQQRVSKTAADNNNSDYSWTGATKYHLNGRDSLVNPGT
AMASHKDDEEKYFPQSGVLIFGKQDSGKTNVDIERVMITDEEEIRTTNPVATEQYGSVS
TNLQSGNTQAATSDVNTQGVLPGMVWQDRDVYLQGPIWAKIPHTDGHFHPSPLMGGF
GLKHPPPQILIKNTPVPANPSTTFSAAKFASFITQYSTGQVSVEIEWELQKENSKRWNPEI
QYTSNYNKSVNVDFTVDTNGVYSEPRPIGTRYLTRNL
>SEQ ID NO: 69 Human Schnurri-3 nucleotide sequence
CTCACAACCAGCCGACTCTCCCATTATCCAGCTGCCTAGTTTGGTGCTTCAATGTACA
TGGCTATTCCGTGTGCATATGTGTGTATACAAACACGCATGCATGCCTGGATGGACATA
CGTATGCACAGGTTATTTTTTAAGGACAATTCTTTCAATAAGGTCTTTACCCCTTACTT
GAAACAGGTGTTCATGAAAAAAATGCACAAAATCCTGCCTGGCCGGAATAATTCATG
AAGAAGGGGCTGGATCCGTGGGTCAGAGAACACAGGACCAGTTTGCCATCCCAAGG
CCGAAGGATTCGAGGCACAAACCCAGCAGCCTCAACCTAGTTCATGGAGGAGCCTC
GCGGGGTCCTGGCCAAGCAAGCCCGCCCCTCTGGTGGGAAGAGCGGCGCCTAGGTG
GAGGGTGGCTGCCGTAGGAGTGGACATGAATGCTGGCTTTCAGAGAGAACAGCGTT
TCAGTTTTGGTCATCGGAAGTGGTGCCTTCAGCACAGAAGAAGAGCGTGATTTCTCC
TCCAAGGCCGTTGATCTCCAACCCAGAACTAAAGGGGAGAAGAGCCACCCCCAGCA
TCCAGCGTGGCATCTCTTGTGCCAGGACCAGGGATGACTGGGCCATGGACACAGATG
TCTCCAACCTTCAACCGTTTGCATAGCACACGGGGGACTCGTGGGGGCCACCTGCCA
CTGCCAGCTGAAACAATACAATGGCAATACTGACATCCTTCATGACGTTTTCCCGACA
GACATTCAGGCAGAAAGTGCTGGTGCGTTTTCTGTCTGCAAAGTAGAGGGCCATGCC
TCACCAATAGAATAGCGTGGGCCCTGATGACCTGCTCCGAGTCCACTCACAGCCAGT
GACACTTGCAAAAAACTCCCAAAGCCGTCTTGGGTTTGGCTCCCACAGCTCTTGACC
AATGTGGCCAAAGCTGGACACCTCCTTGGGACACTGGGATTATTCATAAATGCAGCC
CGCCCTGACTCTCCCTGAATAGCATCTGAAGTCTTTGTGAAGGTCATGGATCCTGAAC
AAAGTGTCAAGGGCACCAAGAAGGCTGAGGGAAGTCCCCGGAAGCGGCTGACCAA
AGGAGAGGCCATTCAGACCAGTGTTTCTTCCAGCGTCCCATACCCAGGCAGCGGCAC
AGCTGCCACCCAAGAGAGCCCCGCCCAAGAGCTCTTAGCCCCGCAGCCCTTCCCGG
GCCCCTCATCAGTTCTTAGGGAAGGCTCTCAGGAGAAAACGGGCCAGCAGCAGAAG
CCCCCCAAAAGGCCCCCCATCGAAGCATCCGTCCACATCTCACAGCTTCCGCAGCAC
CCTCTGACACCAGCATTCATGTCGCCTGGCAAACCTGAGCATCTCCTGGAGGGGTCC
ACATGGCAACTGGTTGACCCCATGAGACCTGGACCCTCTGGCTCCTTCGTGGCCCCT
GGGCTCCATCCTCAGAGCCAGCTCCTTCCTTCCCACGCTTCCATCATTCCCCCCGAGG
ACCTTCCTGGAGTCCCCAAAGTCTTCGTGCCTCGTCCTTCCCAGGTCTCCTTGAAGC
CCACAGAAGAGGCACACAAGAAGGAGAGGAAGCCCCAGAAGCCAGGCAAGTACAT
CTGCCAGTACTGCAGCCGGCCCTGTGCCAAGCCCAGCGTGCTCCAGAAGCACATTCG
CTCACACACAGGTGAGAGGCCCTACCCCTGCGGCCCCTGTGGCTTCTCCTTCAAGAC
CAAGAGTAATCTCTACAAGCACAGGAAGTCCCATGCCCACCGCATCAAAGCAGGCCT
GGCCTCAGGCATGGGTGGCGAGATGTACCCACATGGGCTGGAGATGGAGCGGATCCC
TGGGGAAGAGTTTGAGGAGCCCACTGAGGGAGAAAGCACAGATTCTGAAGAGGAG
ACTAGTGCCACCTCTGGTCACCCTGCAGAGCTCTCCCCAAGACCCAAGCAGCCCCTT
CTCTCCAGCGGGCTATACAGCTCTGGGAGCCACAGTTCCAGCCACGAACGCTGTTCC
CTGTCCCAGTCCAGCACAGCCCAGTCACTCGAAGACCCCCCTCCATTTGTGGAACCC
TCATCTGAGCACCCCCTGAGCCATAAACCTGAAGACACCCACACGATTAAGCAGAAG
CTGGCCCTCCGCTTAAGCGAGAGGAAGAAGGTGATCGATGAGCAGGCGTTTCTGAG
CCCAGGCAGCAAAGGGAGTACTGAGTCTGGGTATTTCTCTCGCTCCGAGAGTGCAGA
GCAGCAGGTCAGCCCCCCAAACACCAACGCCAAGTCCTACGCTGAGATCATCTTTGG
CAAGTGTGGGCGAATAGGACAGCGGACCGCCATGCTGACAGCCACCTCCACCCAGC
CCCTCCTGCCCCTGTCCACCGAAGACAAGCCCAGCCTGGTGCCTTTGTCTGTACCCC
GGACGCAGGTGATCGAGCACATCACGAAGCTCATCACCATCAACGAGGCCGTGGTG
GACACCAGCGAGATCGACAGCGTGAAGCCAAGGCGGAGCTCACTGTCCAGGCGCA
GCAGCATGGAGTCCCCAAAATCCAGCCTCTACCGGGAGCCCCTGTCATCCCACAGTG
AGAAAACCAAGCCTGAACAATCACTGCTGAGCCTCCAGCACCCGCCCAGTACCGCC
CCCCCTGTGCCTCTCCTGAGAAGCCACTCAATGCCTTCTGCCGCCTGCACTATCAGCA
CCCCCCACCACCCCTTCCGAGGTAGCTACTCCTTCGATGACCATATCACCGACTCCGA
AGCCCTGAGCCACAGCAGTCACGTGTTTACCTCCCACCCCCGGATGCTGAAGCGCCA
GCCGGCAATCGAATTACCTTTGGGAGGGGAATACAGTTCTGAGGAGCCTGGCCCAAG
CAGCAAAGACACAGCCTCCAAGCCCTCGGACGAAGTGGAACCCAAGGAAAGCGAG
CTTACCAAAAAGACCAAGAAGGGTTTGAAAACAAAAGGGGTGATCTACGAATGTAA
CATATGTGGTGCTCGGTACAAGAAAAGGGATAACTACGAAGCCCACAAAAAATACTA
CTGCTCAGAGCTTCAGATCGCAAAGCCCATCTCTGCAGGCACCCACACATCTCCAGA
AGCTGAAAAGAGTCAGATTGAGCATGAGCCGTGGTCCCAAATGATGCATTACAAACT
GGGAACCACCCTGGAACTCACTCCACTGAGGAAGAGGAGGAAAGAGAAGAGCCTT
GGGGACGAGGAAGAGCCACCTGCCTTTGAGTCCACAAAAAGTCAGTTTGGCAGCCC
CGGGCCATCTGATGCTGCTCGGAACCTTCCCCTGGAGTCCACCAAGTCACCAGCAGA
ACCAAGTAAATCAGTGCCCTCCTTGGAGGGACCCACGGGCTTCCAGCCAAGGACTC
CCAAGCCAGGGTCCGGTTCAGAATCAGGGAAGGAGAGGAGAACAACGTCCAAAGA
AATTTCTGTCATCCAGCACACCAGCTCCTTTGAGAAATCTGATTCTCTCGAGCAGCCG
AGTGGCTTGGAAGGGGAAGACAAACCTCTGGCCCAGTTCCCATCACCCCCACCTGC
CCCACACGGACGCTCTGCTCACTCCCTGCAGCCTAAGTTGGTCCGCCAGCCCAACAT
TCAGGTTCCTGAGATCCTAGTAACTGAGGAGCCTGACCGGCCGGACACAGAGCCAG
AGCCGCCCCCTAAGGAACCTGAGAAGACTGAGGAGTTCCAATGGCCCCAGCGCAGC
CAGACACTTGCCCAGCTCCCAGCTGAGAAGCTGCCACCCAAAAAGAAGAGGTTGCG
CCTGGCAGAGATGGCCCAATCATCAGGGGAGTCCAGCTTCGAGTCCTCTGTGCCTCT
GTCTCGCAGCCCGAGCCAGGAAAGCAATGTCTCTTTGAGTGGGTCCAGCCGCTCAG
CCTCGTTTGAGAGGGATGACCATGGGAAAGCCGAGGCCCCCAGTCCCTCATCTGACA
TGCGCCCCAAACCCCTGGGCACCCACATGTTGACTGTCCCCAGCCACCACCCACATG
CCCGAGAGATGCGGAGGTCAGCCTCAGAGCAGAGCCCCAACGTTTCCCATTCTGCCC
ACATGACCGAGACACGCAGCAAATCCTTTGACTATGGCAGCTTGTCCTTGACAGGCC
CTTCTGCTCCAGCCCCAGTGGCTCCACCAGCGCGGGTGGCCCCGCCAGAGAGAAGA
AAATGCTTCTTGGTGAGACAGGCCTCTCTGAGCAGGCCTCCAGAATCTGAGTTGGAG
GTTGCCCCCAAGGGAAGACAGGAGAGCGAAGAACCACAGCCCTCATCCAGTAAACC
CTCTGCCAAAAGCTCATTGTCCCAGATTTCCTCTGCGGCCACCTCACATGGTGGACCC
CCGGGAGGCAAGGGCCCAGGGCAGGACAGGCCCCCATTGGGGCCCACTGTGCCCTA
CACAGAAGCACTGCAAGTGTTCCACCACCCCGTTGCCCAGACACCCCTGCATGAGA
AGCCATACCTGCCCCCACCAGTCTCCCTTTTCTCCTTCCAGCATCTCGTGCAGCATGA
GCCAGGACAGTCTCCAGAATTCTTCTCCACCCAGGCCATGTCCAGCCTCCTGTCCTC
ACCATACTCCATGCCCCCACTTCCTCCCTCCTTATTTCAAGCCCCACCGCTTCCTCTCC
AGCCTACTGTTCTGCACCCAGGCCAACTCCATCTCCCCCAGCTCATGCCTCACCCAGC
CAACATCCCCTTCAGGCAGCCCCCTTCCTTCCTCCCCATGCCATACCCGACCTCCTCA
GCACTGTCTTCTGGGTTTTTCCTGCCTCTGCAATCCCAGTTTGCACTTCAGCTCCCTG
GTGATGTGGAAAGCCATCTGCCCCAGATCAAAACCAGCCTGGCCCCACTGGCAACA
GGAAGTGCTGGCCTCTCCCCCAGCACAGAGTACAGCAGTGACATCCGGCTACCCCCT
GTGGCTCCCCCAGCCAGCTCCTCAGCACCTACATCAGCTCCTCCACTGGCCCTGCCT
GCCTGTCCAGACACCATGGTGTCCCTGGTTGTGCCTGTCCGTGTTCAGACCAATATGC
CGTCCTATGGGAGCGCAATGTACACCACCCTTTCCCAGATCTTGGTCACCCAGTCCCA
AGGCAGCTCAGCAACTGTGGCACTTCCCAAGTTTGAGGAACCCCCATCAAAGGGGA
CGACTGTATGTGGTGCAGATGTGCATGAGGTTGGGCCCGGCCCTTCTGGGTTAAGTG
AAGAGCAAAGCAGAGCTTTCCCAACTCCATACCTGAGAGTGCCTGTGACATTACCTG
AAAGAAAAGGCACTTCCCTGTCATCAGAGAGTATCTTGAGCCTGGAGGGGAGTTCAT
CAACAGCAGGGGGAAGCAAACGTGTCCTTTCACCAGCTGGCAGCCTTGAACTTACC
ATGGAAACCCAGCAGCAAAAAAGAGTGAAGGAGGAGGAGGCTTCCAAGGCAGATG
AAAAACTTGAGCTGGTAAAACCATGCAGTGTGGTCCTACCAGCACCGAGGATGGGA
AGAGGCCAGAGAAATCCCACTTAGGCAACCAGGGCCAAGGCAGGAGGGAGCTAGA
AATGCTGTCCAGCCTGTCCTCAGATCCATCTGACACAAAGGAAATTCCTCCCCTCCCT
CACCCTGCATTGTCCCATGGGACAGCCCCAGGCTCAGAAGCTTTGAAGGAATATCCC
CAGCCATCTGGCAAACCTCACCGAAGAGGGTTGACCCCACTGAGCGTGAAGAAAGA
AGATTCCAAGGAACAACCTGATCTCCCCTCCTTGGCACCTCCGAGCTCTCTGCCTCT
GTCAGAAACGTCCTCCAGACCAGCCAAGTCACAAGAAGGTACGGACTCAAAGAAG
GTACTGCAGTTCCCCAGCCTCCACACAACCACTAATGTCAGTTGGTGCTATTTAAACT
ACATTAAGCCAAATCACATCCAGCATGCAGATAGGAGGTCCTCTGTTTACGCTGGTTG
GTGCATAAGTTTGTACAACCCCAACCTTCCGGGGGTTTCCACTAAAGCTGCTTTGTCC
CTCCTGAGGTCTAAGCAGAAAGTGAGCAAAGAGACATACACCATGGCCACAGCTCC
GCATCCTGAGGCAGGAAGGCTTGTGCCATCCAGCTCCCGCAAGCCCCGCATGACAG
AGGTTCACCTCCCTTCACTGGTTTCCCCGGAAGGCCAGAAAGATCTAGCTAGAGTGG
AGAAGGAAGAAGAGAGGAGAGGGGAGCCGGAGGAGGATGCTCCTGCCTCCCAGAG
AGGGGAGCCGGCGAGGATCAAAATCTTCGAAGGAGGGTACAAATCAAACGAAGAGT
ATGTATATGTGCGAGGCCGCGGCCGAGGGAAATATGTTTGTGAGGAGTGTGGAATTC
GCTGCAAGAAGCCCAGCATGCTGAAGAAACACATCCGCACCCACACTGACGTCCGG
CCCTATGTGTGCAAGCACTGTCACTTTGCTTTTAAAACCAAAGGGAATCTGACTAAG
CACATGAAGTCGAAGGCCCACAGCAAAAAGTGCCAAGAGACAGGGGTGCTGGAGG
AGCTGGAAGCCGAAGAAGGAACCAGTGACGACCTGTTCCAGGACTCGGAAGGACG
AGAGGGTTCAGAGGCTGTGGAGGAGCACCAGTTTTCGGACCTGGAGGACTCGGACT
CAGACTCAGACCTGGACGAAGACGAGGATGAGGATGAGGAGGAGAGCCAGGATGA
GCTGTCCAGACCATCCTCAGAGGCGCCCCCGCCTGGCCCACCACATGCACTGCGGGC
AGACTCCTCACCCATCCTGGGCCCTCAGCCCCCAGATGCCCCCGCCTCTGGCACGGA
GGCTACACGAGGCAGCTCGGTCTCGGAAGCTGAGCGCCTGACAGCCAGCAGCTGCT
CCATGTCCAGCCAGAGCATGCCGGGCCTCCCCTGGCTGGGACCGGCCCCTCTGGGCT
CTGTGGAGAAAGACACAGGCTCAGCCTTGAGCTACAAGCCTGTGTCCCCAAGAAGA
CCGTGGTCCCCAAGCAAAGAAGCAGGCAGCCGTCCACCACTAGCCCGCAAACACTC
GCTAACCAAAAACGACTCATCTCCCCAGCGATGCTCCCCGGCCCGAGAACCACAGG
CCTCAGCCCCAAGCCCACCTGGCCTGCACGTGGACCCAGGAAGGGGCATGGGCGCT
CTCCCTTGTGGGTCTCCAAGACTTCAGCTGTCTCCTCTCACCCTCTGCCCCCTGGGAA
GAGAACTGGCCCCTCGAGCACATGTGCTCTCCAAACTCGAGGGTACCACCGACCCA
GGCCTCCCCAGATACTCGCCCACCAGGAGATGGTCTCCAGGTCAGGCCGAGTCACCA
CCACGGTCAGCGCCGCCAGGGAAGTGGGCCTTGGCTGGGCCGGGCAGCCCCTCAGC
GGGGGAGCATGGCCCAGGCTTGGGGCTGGACCCACGGGTTCTCTTCCCGCCCGCGC
CTCTACCTCACAAGCTCCTCAGCAGAAGCCCAGAGACCTGCGCCTCCCCGTGGAAG
GCCGAGTCCCGAAGTCCCTCCTGCTCACCCGGCCCTGCTCATCCTCTCTCCTCCCGAC
CCTTCTCCGCCCTCCATGACTTCCACGGCCACATCCTGGCCCGGACAGAGGAGAACA
TCTTCAGCCACCTGCCTCTGCACTCCCAGCACTTGACCCGTGCCCCATGTCCCTTGAT
TCCCATCGGTGGGATCCAGATGGTGCAGGCCCGGCCAGGAGCCCACCCCACCCTGCT
GCCAGGGCCCACCGCAGCCTGGGTCAGTGGCTTCTCCGGGGGTGGCAGCGACCTGA
CAGGGGCCCGGGAGGCCCAGGAGCGAGGCCGCTGGAGTCCCACTGAGAGCTCGTC
AGCCTCCGTGTCGCCTGTGGCTAAGGTCTCCAAATTCACACTCTCCTCAGAGCTGGA
GGGCGGGGACTACCCCAAGGAGAGGGAGAGGACCGGCGGAGGCCCGGGCAGGCCT
CCTGACTGGACACCCCATGGGACCGGGGCACCTGCAGAGCCCACACCCACGCACAG
CCCCTGCACCCCACCCGACACCTTGCCCCGGCCGCCCCAGGGACGCCGGGCAGCGC
AGTCCTGGAGCCCCCGCTTGGGTCCCCGCGTGCACCGACCAACCCCGAGCCTTCTGC
CACCCCGCCGCTGGACCGCAGCAGCTCTGTGGGCTGCCTGGCAGAGGCCTCTGCCC
GCTTCCCAGCCCGGACGAGGAACCTCTCCGGGGAACCCAGGACCAGGCAGGACTCC
CCCAAGCCCTCAGGAAGTGGGGAGCCCAGGGCACATCCACATCAGCCTGAGGACAG
GGTTCCCCCCAACGCTTAGCCTCTCTCCAACTGCTTCAGCATCTGGCTTCCAGTGTCC
AGCAACAGACGTTTCCAGCCACTTTCCTCGAATCATCCCACTTCCTCAGCCCCATCTG
TCCCTCCGTCCAGGAGCTCTCACGGCCCCATCTGTTGTACCTTCCCATGTATGCAGTT
ACCTGTGCCTTTTTCTACACCTTTTGTTGCTTAAAAAGAAACAAAACAAATCACATAC
ATACATTTAAAAAAAAAACAACAACCCACGAGGAGTCTGAGGCTGTGAATAGTTTAT
GGTTTTGGGGAAAGGCTGATGGTGAAGCCTCCTGACCCTCCCCGCTGTGGTTGGCAG
CCACCCACCCCAGAGGCTGGCAGAGGGAAAGGGGTACACTGAGGGAGAAAGGAAA
AGGAAACTTCAAACAATATAGAATTAAATGTAAAAGGAAGCACTCCTGTGTACAGAT
GCGATCAAGGTTCCTGTTTATTGCCACTTCACCCCCCTGCCCAGCTCGTAGCCACCCC
TCTCTGCCAGCAGAAAGGCCAGTGTCCCCAGGCAGAGGGGCACAAACACAGGCAG
GTGACCCCCACCCAGGCCCCAGCAGGCAGGCCCAGAAAAACTAATCTTTTCCTTTTT
TTTTTTTTTTTTTTTTTTTGCAAGAAAATAAAATGATACTTTTCCTAGGATTTCAACAC
AAAATAATAGGTGCAGGTAGAAGGAGGAGGGCTGGCTCCCCAAGGGCTCCTGGATA
CTCTGGTAGTCTGAGTCATGGGCCCATCCTGGCACTCCACAGGTGGGCAGGCCACCC
CACCCACGCACCCCCACTCCAGACACCTCCCTTCTGCACCCCACCCTGGCCCCCTGG
GCTGGGGAAGGAGCCCTGACTGTCCGTCCCTGGCTCCCAAGCCCCTGACCGAGGCC
TCACTCTCCTGTTGCCTCCTCTGTTCTAAAACCACCAAACCACCCACAAAGGCAGAA
GTGGCAGGGCCCGAGCCCTAGCGGCCGTTCCTGAGACTGGGTTTTGGGTTTTGTTTC
ATCTTGGTCCCTGGGGTACAAGGGAGCCTGTTCCCCTCATGGCTGGGTTTTTCCAGTT
CTCCACAGCAGAGGTTTGCGGGGAACTGTTTCAGGACCACTTTGCCACAGGACCGT
TTCCCCCCGTCCCTGCCCCTGTCTCCACTACCCCAAGGAAATACCCACAACTGTGGCT
GGTGGATACGGCCTGGACCTGTTTGCTGTCTTACACCTCTTTTTTAAAAAGAGAGAG
GATGGTGTTTGATACTTCACCCAGCCACCACAGATTCTTTTGACCTAGAGGATTTTTG
AATTGTCCTAACTCGTTGGAATTCTCCAAAGCAATCAGTGTGAGCCAGTGCCTCTTCC
TTACCCACATCTCTACTTTCAAGAAGCTGCCCTGCATTTCCTGGGGCAAAACTCTACT
TTGTAAGAAAAATAATAGGACCAGAAATTTAAATCCCAAATTGAACTATGGAACTTGA
ACTCTAGCGTGTTCGCCCCAACTGGGAGAGGTGAGCTTTTTCCCAGTGTTTCAGAAC
TGATTTTCTTTACTTTCTACAAGGGAGGGCAGCACAGGGACTACGGTTGAGGCCCGT
GAAGGCTGGGTTTGATGCCACCCTATACAGAGCAGGGACCTCTCTGGCTAATCCCCA
GTCCTCAGCCAGGCTGTGTGAATCAAGTGCCTGCCCCAGGGCTCTTGAGCTATTGAA
GCTGCTTGGGTACAGGACACAGTAGGTGGGGAGGGTTAAGACCCTTCTGTGAGTTCC
CTGTGCGGGGCTGTACTTGCCTCTTCCAATTCGTGGCCTTTCCCTGCTTGGTCCCTACT
AGACAGACAAACCAGCCACAGTCCAGCCTGCAGCCAGACCACCTTGTTCACTCATT
CTCCTTTGCCTCAGAGCTAAGACAAAAATGAGACAGAAGGCAGGGCTCCCTGGGAG
TCCACTGTGCTCCAGGGTTCTGGGGAATCAGGGTTAGCCAGCAGCTCCTGGCTGCTT
CCCTCAGAGACTAGGGCTCTCATCCTCCCCAAGAGAAGCAGCAAGCCCAGCCTGGA
CCACACTGTCCATATTGCTGGACAGTGGCCTGACAGAAAGTGACTCCTCCAAGTCCC
AGGAGGCCAGGGCTTTTCTCATCCTTGCCTTTCAGCCCTAACCCATGGGACTGCCCA
CGGATTGGAGACTTCAAGGGCTGAGGTCTGGGAGCTGCATAAAGGGCATTGCTTCAG
CCCAGGTTAGAAATCTGCCTGGGCAAGCTCTTCCTGCCCCAGACCTACAAAGCAGCA
GACCGGGGGCTCTGGTGGACTAGCCCCTGACATTGGTGGGGGGCCCCACACCACTC
CACCCCACCCTGCCTTCCAGCTCTCCTGGGCATTTTTCTCCCTGTACTCAAACAGCCT
ACCCACCCAAGGTTTCCTCCCTGGGCAGCCTAGCAATGAACAGTGCAGCCGGCAGG
GCAGAGGCCCGGCAGTCACCGGGCCCGTCAGGCTCAGGCAGAGAAGCCACAGGGG
CCAGGAGTCACTGGAGACTATTTCTAAATGATGGGGGTAAATGCACAAATAGAATCTC
ACCAAAGGGCTGCCTCCACATTGATGCCGTGCCCAGAGGGACAGAACCAATGCCAC
CAGCCTGGGTATATGTCACTGGGCACAGCTCTAACCCCCTCCTCCGGACTCTAGTCCC
GCTCCTCTGCGCACAGAGCCCCCAGCCCACAGGTACACCTTCATGATTTGGAGAAAG
ACGCTCGCCCCATGCACGCCCTCCTCTGGGCCTTCTGCCCTGCTCCCAGTCACTTCCA
AGCTTCCTGTTTGCCTGTGATGTTATTGTGCCTGTTGAGGGAAGCAGCAGAGGAGGC
AGTGGCTGACTTGGCACAGATGCCTGCTACGTGCTCTGTTGAAATGCGCGGGGTGGC
CATTCCTCGGTACAGACTAGTCCTGGTCCTTGGGTGTGGGCAGTGGGGGAGGAACCA
ACTGGTCGAGGTTTCAGAGCCAAACCTTGCCTTTGGTTGGTGAGTCCTTGCCCCCCA
GGCCTGCGCTCCACGATGCCTTTCACCCTTGGCAATCTCAGGGCCATCCTGGGTAGTA
ACCCCACTCCTCTCTGCTCCCGCCCGCACCTGTGGCTCTCACTCTGGGCTCAACCCCT
GCAACCCTCCAGGAGCCCGACAGCAGCCAGCTGCCTGCACTGTCGCCTCCGTAAGC
TCCAACTTCCAGACCCAGAAGTCCCTCTGCTTCCCTCTGTTGGAAAAAGCCTAAAAG
AATTAGCTTCCAGATTCCTCTAGCCCCTGCTCCATTCCCACCCAGTCCTTCTGAAGAG
GAATGAGCAATACATCTGAGCTGGATTTCTCTCTAGTCCTTTCTCCAGACAAATCCTT
CTTAAAGCAAAAGTCCTGGCTGAGCACCTGTCCTTGGGGACCGATCTGCCGTGTGAC
CAGGGGAAGAAAGTTCCCGAAAGCCTGTTCCACCAATTCTGCTTCTGTGTTGTGAAT
CCAGTCTGCTTTCCATTAGAAAACCGCTTCGGCACTTATGGTCACTTTAATAAATCTA
GTATGTAAAAAAAGAAAGAAAGAAAAGAAACAGAAAAAAGAAACGTGCAGGCAAA
TGTAAAATACAATGCTCTCTGTAAGATAAATATTTGCCTTTTTTTCTAAAAGGTGTACG
TATTCTGTATGTGAAATTGTCTGTAGAAAGTTTCTATGTTCTTAAATGGCAATACATTC
CAAAAATTGTACTGTAGATATGTACAGCAACCGCACTGGGATGGGGTAGTTTTGCCTG
TAATTTTATTTAAACTCCAGTTTCCACACTTGCTCTTGCAATGTTGGTATGGTATATATC
AGTGCAAAAGAAAAAACAAAACAGAAACAAACAAAAAAAAAAAACAAAAATCCA
CGCAGGTCTAAAGCACAGAGTCTGACGTACAAAAGGAAAAATGCTCAGTATTGATGT
GTGTGACCTTTGTTGTAAATTACATCTGTACTGTGAATGAGAAGTTTTTACAAGTATAA
TAATTGCCTTTATTACAGCTCTGGCTGAGTGTTCAGCCTGAGGATATTTTTTAAAAAAA
AAAGAATTAGCATGTTGGAATAAATTTGAAAATCCCAACATAAAAAAA
>SEQ ID NO: 70 Human Schnurri-3 amino acid sequence
MDPEQSVKGTKKAEGSPRKRLTKGEAIQTSVSSSVPYPGSGTAA
TQESPAQELLAPQPFPGPSSVLREGSQEKTGQQQKPPKRPPIEASVHISQLPQHPLTP
AFMSPGKPEHLLEGSTWQLVDPMRPGPSGSFVAPGLHPQSQLLPSHASIIPPEDLPGV
PKVFVPRPSQVSLKPTEEAHKKERKPQKPGKYICQYCSRPCAKPSVLQKHIRSHTGER
PYPCGPCGFSFKTKSNLYKHRKSHAHRIKAGLASGMGGEMYPHGLEMERIPGEEFEEP
TEGESTDSEEETSATSGHPAELSPRPKQPLLSSGLYSSGSHSSSHERCSLSQSSTAQS
LEDPPPFVEPSSEHPLSHKPEDTHTIKQKLALRLSERKKVIDEQAFLSPGSKGSTESG
YFSRSESAEQQVSPPNTNAKSYAEIIFGKCGRIGQRTAMLTATSTQPLLPLSTEDKPS
LVPLSVPRTQVIEHITKLITINEAVVDTSEIDSVKPRRSSLSRRSSMESPKSSLYREP
LSSHSEKTKPEQSLLSLQHPPSTAPPVPLLRSHSMPSAACTISTPHHPFRGSYSFDDH
ITDSEALSHSSHVFTSHPRMLKRQPAIELPLGGEYSSEEPGPSSKDTASKPSDEVEPK
ESELTKKTKKGLKTKGVIYECNICGARYKKRDNYEAHKKYYCSELQIAKPISAGTHTS
PEAEKSQIEHEPWSQMMHYKLGTTLELTPLRKRRKEKSLGDEEEPPAFESTKSQFGSP
GPSDAARNLPLESTKSPAEPSKSVPSLEGPTGFQPRTPKPGSGSESGKERRTTSKEIS
VIQHTSSFEKSDSLEQPSGLEGEDKPLAQFPSPPPAPHGRSAHSLQPKLVRQPNIQVP
EILVTEEPDRPDTEPEPPPKEPEKTEEFQWPQRSQTLAQLPAEKLPPKKKRLRLAEMA
QSSGESSFESSVPLSRSPSQESNVSLSGSSRSASFERDDHGKAEAPSPSSDMRPKPLG
THMLTVPSHHPHAREMRRSASEQSPNVSHSAHMTETRSKSFDYGSLSLTGPSAPAPVA
PPARVAPPERRKCFLVRQASLSRPPESELEVAPKGRQESEEPQPSSSKPSAKSSLSQI
SSAATSHGGPPGGKGPGQDRPPLGPTVPYTEALQVFHHPVAQTPLHEKPYLPPPVSLF
SFQHLVQHEPGQSPEFFSTQAMSSLLSSPYSMPPLPPSLFQAPPLPLQPTVLHPGQLH
LPQLMPHPANIPFRQPPSFLPMPYPTSSALSSGFFLPLQSQFALQLPGDVESHLPQIK
TSLAPLATGSAGLSPSTEYSSDIRLPPVAPPASSSAPTSAPPLALPACPDTMVSLVVP
VRVQTNMPSYGSAMYTTLSQILVTQSQGSSATVALPKFEEPPSKGTTVCGADVHEVGP
GPSGLSEEQSRAFPTPYLRVPVTLPERKGTSLSSESILSLEGSSSTAGGSKRVLSPAG
SLELTMETQQQKRVKEEEASKADEKLELVKPCSVVLTSTEDGKRPEKSHLGNQGQGRR
ELEMLSSLSSDPSDTKEIPPLPHPALSHGTAPGSEALKEYPQPSGKPHRRGLTPLSVK
KEDSKEQPDLPSLAPPSSLPLSETSSRPAKSQEGTDSKKVLQFPSLHTTTNVSWCYLN
YIKPNHIQHADRRSSVYAGWCISLYNPNLPGVSTKAALSLLRSKQKVSKETYTMATAP
HPEAGRLVPSSSRKPRMTEVHLPSLVSPEGQKDLARVEKEEERRGEPEEDAPASQRGE
PARIKIFEGGYKSNEEYVYVRGRGRGKYVCEECGIRCKKPSMLKKHIRTHTDVRPYVC
KHCHFAFKTKGNLTKHMKSKAHSKKCQETGVLEELEAEEGTSDDLFQDSEGREGSEAV
EEHQFSDLEDSDSDSDLDEDEDEDEEESQDELSRPSSEAPPPGPPHALRADSSPILGP
QPPDAPASGTEATRGSSVSEAERLTASSCSMSSQSMPGLPWLGPAPLGSVEKDTGSAL
SYKPVSPRRPWSPSKEAGSRPPLARKHSLTKNDSSPQRCSPAREPQASAPSPPGLHVD
PGRGMGALPCGSPRLQLSPLTLCPLGRELAPRAHVLSKLEGTTDPGLPRYSPTRRWSP
GQAESPPRSAPPGKWALAGPGSPSAGEHGPGLGLDPRVLFPPAPLPHKLLSRSPETCA
SPWKAESRSPSCSPGPAHPLSSRPFSALHDFHGHILARTEENIFSHLPLHSQHLTRAP
CPLIPIGGIQMVQARPGAHPTLLPGPTAAWVSGFSGGGSDLTGAREAQERGRWSPTES
SSASVSPVAKVSKFTLSSELEGGDYPKERERTGGGPGRPPDWTPHGTGAPAEPTPTHS
PCTPPDTLPRPPQGRRAAQSWSPRLESPRAPTNPEPSATPPLDRSSSVGCLAEASARFPAR
TRNLSGEPRTRQDSPKPSGSGEPRAHPHQPEDRVPPNA
>SEQ ID NO: 55; amiR-33-mRANKi-1:
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGAATGGTCCA
CATTTCAGGGACTGTTCTGGCAATACCTGGTCCCTGATTTATGGACCATTCACGGAGG
CCTGCCCTGACTGCCCACGGTGCCGTGGCCAAAGAGGATCTAAGGGCACCGCTGAG
GGCCTACCTAACCATCGTGGGGAATAAGGACAGTGTCACCC
>SEQ ID NO: 56; amiR-33-mRANKi-2:
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGACAAATTAG
CTGTCAGCGCTGTTCTGGCAATACCTGGCGCTGACTGCAAATTTGTCACGGAGGCCT
GCCCTGACTGCCCACGGTGCCGTGGCCAAAGAGGATCTAAGGGCACCGCTGAGGGC
CTACCTAACCATCGTGGGGAATAAGGACAGTGTCACCC
>SEQ ID NO: 57; Bone-resorbing peptide (8-mer)
DDDDDDDD
>SEQ ID NO: 58; Bone-resorbing peptide (9-mer)
DDDDDDDDD
>SEQ ID NO: 59; Bone-resorbing peptide (10-mer)
DDDDDDDDDD
>SEQ ID NO: 60; Bone-resorbing peptide (11-mer)
DDDDDDDDDDD
>SEQ ID NO: 61; Bone-resorbing peptide (12-mer)
DDDDDDDDDDDD
>SEQ ID NO: 62; Bone-resorbing peptide (13-mer)
DDDDDDDDDDDDD
>SEQ ID NO: 63; Bone-resorbing peptide (14-mer)
DDDDDDDDDDDDDD

1. An isolated nucleic acid encoding:
(i) a first region comprising a first adeno-associated virus (AAV) inverted terminal repeat (ITR), or a variant thereof; and,
(ii) a second region comprising a transgene encoding at least one bone metabolism modulating agent.

2. The isolated nucleic acid of claim 1, wherein the bone metabolism modulating agent is a bone formation promoting agent, optionally wherein the bone formation promoting agent is selected from the group consisting of a protein that promotes osteoblast and/or osteocyte function or activity, a protein that inhibits osteoclast function, and an inhibitory nucleic acid that inhibits osteoclast expression or activity.

3. The isolated nucleic acid of claim 1, wherein the bone metabolism modulating agent is a bone formation inhibiting agent, optionally wherein the bone formation inhibiting agent is selected from the group consisting of a protein that inhibits osteoblast and/or osteocyte function or activity, a protein that promotes osteoclast function or activity, and an inhibitory nucleic acid that inhibits osteoblast expression or activity.

4. The isolated nucleic acid of claim 2, wherein the transgene encodes a bone formation promoting agent selected from the group consisting of parathyroid hormone (PTH), PTH-related protein (PTHrP), deglycase DJ1, an inhibitory nucleic acid targeting sclerostin (SOST), an inhibitory nucleic acid targeting schnurri-3 (SHN3), an inhibitory nucleic acid targeting cathepsin K (CTSK), and an inhibitory nucleic acid targeting receptor activator of NF-κβ (RANK).

5. The isolated nucleic acid of claim 3, wherein the transgene encodes a bone formation inhibiting agent selected from the group consisting of sclerostin (SOST), schnurri-3 (SHN3), cathepsin K (CTSK), an inhibitory nucleic acid targeting parathyroid hormone (PTH), an inhibitory nucleic acid targeting PTH-related protein (PTHrP), and an inhibitory nucleic acid targeting deglycase DJ1.

6. The isolated nucleic acid of claim 1, wherein the transgene encodes at least one inhibitory nucleic acid selected from the group consisting of dsRNA, siRNA, shRNA, miRNA, and artificial miRNA (amiRNA).

7. The isolated nucleic acid of claim 6, wherein the inhibitory nucleic acid functions as a mutant terminal repeat (mTR).

8-16. (canceled)

17. A recombinant adeno-associated virus (rAAV) comprising:
(i) a capsid protein; and,
(ii) the isolated nucleic acid of claim 1;
wherein the capsid protein comprises a heterologous bone-targeting peptide.

18. The rAAV of claim 17, wherein the heterologous bone-targeting peptide comprises the amino acid sequence set forth in SEQ ID NO: 16, 17, 57, 58, 59, 60, 61, 62, or 63.

19. The rAAV of claim 17, wherein a nucleic acid sequence encoding the heterologous bone-targeting peptide is inserted into a VP2 open reading frame of the capsid protein, optionally wherein the nucleic acid sequence is inserted between codons corresponding to N587 and R588 of a nucleic acid sequence encoding an AAV9 capsid protein.

20. A recombinant adeno-associated virus (rAAV) comprising:
(i) a capsid protein; and,
(ii) the isolated nucleic acid of claim 1;
wherein the capsid protein is encoded by an amino acid sequence having one or more azide-bearing unnatural amino acids.

21. The rAAV of claim 20, wherein the capsid protein is conjugated to one or more alendronate (Ale) moiety via the one or more azide-bearing unnatural amino acids.

22. The rAAV of any one of claims 17-21, wherein the rAAV is a self-complementary AAV (scAAV).

23. An isolated nucleic acid encoding a recombinant adeno-associated virus (rAAV) capsid protein comprising a heterologous bone-targeting peptide, wherein the heterologous bone-targeting peptide comprises the amino acid sequence set forth in SEQ ID NO:
16,17, 57, 58, 59, 60, 61, 62, or 63.

24. A recombinant AAV capsid protein comprising one or more azide-bearing unnatural amino acids, wherein the capsid protein is conjugated to one or more alendronate (Ale) moiety via the one or more azide-bearing unnatural amino acids.

25. A method for delivering a transgene to bone tissue, the method comprising administering to a subject the isolated nucleic acid of claim 1.

26. A method for treating a disease or disorder associated with reduced bone density, the method comprising administering to a subject having or suspected of having a disease or disorder associated with reduced bone density the rAAV of claim 17, wherein the transgene encodes a bone formation promoting agent, optionally wherein the bone formation promoting agent is selected from the group consisting of a protein that promotes osteoblast and/or osteoclast function or activity, a protein that inhibits osteoclast function or activity, and an inhibitory nucleic acid that inhibits osteoclast expression or activity.

27. The method of claim 26, wherein the bone formation promoting agent selected from the group consisting of parathyroid hormone (PTH), PTH-related protein (PTHrP), deglycase DJ1, an inhibitory nucleic acid targeting sclerostin (SOST), an inhibitory nucleic acid targeting Schnurri-3 (SHN3), an inhibitory nucleic acid targeting cathepsin K (CTSK), and an inhibitory nucleic acid targeting receptor activator of NF-κβ (RANK).

28. The method of claim 26, wherein the disease or disorder associated with reduced bone density is osteoporosis, a critical sized-bone defect, a mechanical disorder resulting from disuse or injury, and secondary disorders such as breast cancer or prostate cancer metastasis, type 1 diabetes, lupus, rheumatoid arthritis, inflammatory bowel disease, hyperthyroidism, celiac disease, asthma, multiple sclerosis, and periodontitis.

29. A method for treating a disease or disorder associated with deformed and exaggerated (e.g., increased) bone density, the method comprising administering to a subject having or suspected of having a disease or disorder associated with increased bone density the rAAV of claim 17, wherein the transgene encodes a bone formation inhibiting agent, optionally wherein the bone formation inhibiting agent is selected from the group consisting of a protein that inhibits osteoblast and/or osteocyte function or activity, a protein that promotes osteoclast function or activity, and an inhibitory nucleic acid that inhibits osteoblast expression or activity.

30-34. (canceled)


SEQUENCE LISTING (70 sequences)

SEQ ID NO: 1 - DNA, 327 bp, synthetic construct
  misc_feature 1..327: note = Synthetic Polynucleotide
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgtacaaactacttgagagcaggtgttctggcaatacctgcctgctctgtaatagtttgtacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 2 - DNA, 327 bp, synthetic construct
  misc_feature 1..327: note = Synthetic Polynucleotide
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgactacaggtactcacaagctctgttctggcaatacctggagcttgtctgcacctgtagtcacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 3 - DNA, 325 bp, synthetic construct
  misc_feature 1..325: note = Synthetic Polynucleotide
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgtttccatggtaagttcaaggctgttctggcaatacctggccttgaagatgccatggaaacacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 4 - DNA, 327 bp, synthetic construct
  misc_feature 1..327: note = Synthetic Polynucleotide
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgtttcatcatagtacacacctctgttctggcaatacctggaggtgtgatccatgatgaaacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 5 - DNA, 285 bp, synthetic construct
  misc_feature 1..285: note = Synthetic Polynucleotide
  source 1..285: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgttactgtaggatcgagagggatgttctggcaatacctgtccctctccttactacagtaacacggaggcctgccctgactgcccacggtgccgtggccaaagaggatctaagggcaccgctgagggcctacctaaccatcgtggggaataagg

SEQ ID NO: 6 - DNA, 325 bp, synthetic construct
  misc_feature 1..325: note = Synthetic Polynucleotide
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgattatcgctattgcagctttctgttctggcaatacctggaaagctggtacagcgataatcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 7 - DNA, 325 bp, synthetic construct
  misc_feature 1..325: note = Synthetic Polynucleotide
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgtcagattatcgctattgcagctgttctggcaatacctggctgcaattccaataatctgacacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 8 - DNA, 327 bp, synthetic construct
  misc_feature 1..327: note = Synthetic Polynucleotide
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgacaagtaggcagatgaggcactgttctggcaatacctggtgcctcaagtacctacttgtcacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 9 - DNA, 327 bp, synthetic construct
  misc_feature 1..327: note = Synthetic Polynucleotide
  source 1..327: mol_type = other DNA
tttgtcttttatttcaggtcccagatctagggctctgcgtttgctccaggtagtccgctgctcccttgggcctgggcccactgacagccctggtgcctctggccggctgcacacctcctggcgggcagctgtgtgacctctgtggcatcattcctgttctggcaatacctgggaatgatcgcgcagaggtcacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 10 - DNA, 325 bp, synthetic construct
  misc_feature 1..325: note = Synthetic Polynucleotide
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgatggtcttgttgttctccagctgttctggcaatacctggctggagatgagcaagaccatcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 11 - DNA, 325 bp, synthetic construct
  misc_feature 1..325: note = Synthetic Polynucleotide
  source 1..325: mol_type = other DNA
gtcttttatttcaggtcccagatcttagggctctgcgtttgctccaggtagtccgctgctcccttgggcctgggcccactgacagccctggtgcctctggccggctgcacacctcctggcgggcagctgtgacgtctttggtctcaaaggggtgttctggcaatacctgcccctttgtcatcaaagacgtcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 12 - DNA, 411 bp, Homo sapiens
  source 1..411: mol_type = genomic DNA
ggaattgtacccgcggccgatccaccggtgccaccatgatacctgcaaaagacatggctaaagttatgattgtcatgttggcaatttgttttcttacaaaatcggatgggaaatctgttaagaagagatctgtgagtgaaatacagcttatgcataacctgggaaaacatctgaactcgatggagagagtagaatggctgcgtaagaagctgcaggatgtgcacaattttgttgcccttggagctcctctagctcccagagatgctggttcccagaggccccgaaaaaaggaagacaatgtcttggttgagagccatgaaaaaagtcttggagaggcagacaaagctgatgtgaatgtattaactaaagctaaatcccagtgaagcttatcgataccgtcgactagagctc

SEQ ID NO: 13 - DNA, 591 bp, Homo sapiens
  source 1..591: mol_type = genomic DNA
ggaattgtacccgcggccgatccaccggtgccaccatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggtaaagcttatcgataccgtcgactagagctc

SEQ ID NO: 14 - DNA, 696 bp, Mus musculus
  source 1..696: mol_type = genomic DNA
ggaattgtacccgcggccgatccaccggtcgccaccatgggatggagctgtattatcctgtttctcgtcgctactgccaccggagctcattccgcttccaaaagagctctggtcatcctggccaaaggagcagaggagatggagacagtgattcctgtggatgtcatgcggcgagccgggatcaaagtcactgttgcaggcttggctgggaaggaccccgtgcagtgtagccgtgatgtaatgatttgtccagataccagtctggaagatgcaaaaacgcagggaccatacgatgtggtggttcttccaggaggaaatctgggtgcacagaatttatctgagtcgcctatggtgaaggagatcctcaaggagcaggagagcaggaagggcctcatagctgccatctgtgcaggtcctacggctctgttggctcacgaagtaggttttggatgcaaggtcacaacacacccactggctaaggacaaaatgatgaatggcagtcactacagctactcagagagccgcgtggagaaggacggcctgatcctcaccagccgcgggccggggaccagctttgagtttgcactagccattgtggaggcactcgtggggaaagacatggccaaccaagtgaaggcaccgcttgttctcaaagactagtaaaagcttatcgataccgtcgactagagctcgctg

SEQ ID NO: 15 - DNA, 696 bp, Homo sapiens
  source 1..696: mol_type = genomic DNA
ggaattgtacccgcggccgatccaccggtcgccaccatgggatggagctgtattatcctgtttctcgtcgctactgccaccggagctcattccgcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagtaaaagcttatcgataccgtcgactagagctcgctg

SEQ ID NO: 16 - protein, 18 aa, synthetic construct
  REGION 1..18: note = Synthetic Polypeptide
DSSDSSDSSDSSDSSDSS

SEQ ID NO: 17 - protein, 19 aa, synthetic construct
  REGION 1..19: note = Synthetic Polypeptide
  MOD_RES 2: note = is a gamma-carboxylated glutamic acid
  MOD_RES 6: note = is a gamma-carboxylated glutamic acid
  MOD_RES 9: note = is a gamma-carboxylated glutamic acid
  MOD_RES 11: note = is a gamma-carboxylated glutamic acid
  MOD_RES 15: note = is a gamma-carboxylated glutamic acid
  MOD_RES 18: note = is a gamma-carboxylated glutamic acid
CEPRREVAELEPRREVAEL

SEQ ID NO: 18 - protein, 736 aa, synthetic construct
  REGION 1..736: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 19 - protein, 735 aa, synthetic construct [sequence omitted]

SEQ ID NO: 20 - protein, 736 aa, synthetic construct
  REGION 1..736: note = Synthetic Polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 21 - protein, 734 aa, synthetic construct
  REGION 1..734: note = Synthetic Polypeptide
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 22 - protein, 724 aa, synthetic construct
  REGION 1..724: note = Synthetic Polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 23 - protein, 736 aa, synthetic construct
  REGION 1..736: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 24 - protein, 736 aa, synthetic construct
  REGION 1..736: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 25 - protein, 737 aa, synthetic construct
  REGION 1..737: note = Synthetic Polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 26 - protein, 738 aa, synthetic construct
  REGION 1..738: note = Synthetic Polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - protein, 736 aa, synthetic construct
  REGION 1..736: note = Synthetic Polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 736 aa, synthetic construct
  REGION 1..736: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 29 - protein, 738 aa, synthetic construct
  REGION 1..738: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 30 - protein, 738 aa, synthetic construct
  REGION 1..738: note = Synthetic Polypeptide
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 31 - protein, 737 aa, synthetic construct
  REGION 1..737: note = Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 32 - protein, 735 aa, synthetic construct
  REGION 1..735: note = Synthetic Polypeptide
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 33 - protein, 735 aa, synthetic construct
  REGION 1..735: note = Synthetic Polypeptide
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKLASFITQYSTGQVSVEIEWELQKENSKRWNPEIQYTSNYNKSVNVDFTVDTNGVYSEPRPIGTRYLTRNL

SEQ ID NO: 34 - protein, 735 aa, synthetic construct
  REGION 1..735: note = Synthetic Polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLARAVFQAKKRVLEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQTVGRSSFYCLEYFPSQMLRTGNNFTFSYTFEDVPFHSSYAHSQSLDRLMNPLIDQYLYYLSRTNTPSGTTTQSRLRFSQAGASDIRDQSRNWLPGPCYRQQRVSKTAADNNNSDYSWTGATKYHLNGRDSLVNPGTAMASHKDDEEKYFPQSGVLIFGKQDSGKTNVDIERVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - DNA, 21 bp, synthetic construct
  misc_feature 1..21: note = Synthetic Polynucleotide
  source 1..21: mol_type = other DNA
agaggccattcagacgagtgt

SEQ ID NO: 36 - DNA, 20 bp, synthetic construct
  misc_feature 1..20: note = Synthetic Polynucleotide
  source 1..20: mol_type = other DNA
ctgcggaagctgagagatgt

SEQ ID NO: 37 - DNA, 25 bp, synthetic construct
  misc_feature 1..25: note = Synthetic Polynucleotide
  source 1..25: mol_type = other DNA
cacaatatcaaggatatcgacgtga

SEQ ID NO: 38 - DNA, 25 bp, synthetic construct
  misc_feature 1..25: note = Synthetic Polynucleotide
  source 1..25: mol_type = other DNA
acatcagttctgttcttcgggtaca

SEQ ID NO: 39 - DNA, 25 bp, synthetic construct
  misc_feature 1..25: note = Synthetic Polynucleotide
  source 1..25: mol_type = other DNA
tacaaaccatacccagtccctgttt

SEQ ID NO: 40 - DNA, 24 bp, synthetic construct
  misc_feature 1..24: note = Synthetic Polynucleotide
  source 1..24: mol_type = other DNA
agtgctctaaccacagtccatgca

SEQ ID NO: 41 - DNA, 20 bp, synthetic construct
  misc_feature 1..20: note = Synthetic Polynucleotide
  source 1..20: mol_type = other DNA
cagggaggcagtgactcttc

SEQ ID NO: 42 - DNA, 20 bp, synthetic construct
  misc_feature 1..20: note = Synthetic Polynucleotide
  source 1..20: mol_type = other DNA
agtgtggaaagtgtggcgtt

SEQ ID NO: 43 - DNA, 20 bp, synthetic construct
  misc_feature 1..20: note = Synthetic Polynucleotide
  source 1..20: mol_type = other DNA
atggcgtcctctctgcttga

SEQ ID NO: 44 - DNA, 20 bp, synthetic construct
  misc_feature 1..20: note = Synthetic Polynucleotide
  source 1..20: mol_type = other DNA
gaagggtgggtagtcatttg

SEQ ID NO: 45 - DNA, 20 bp, synthetic construct
  misc_feature 1..20: note = Synthetic Polynucleotide
  source 1..20: mol_type = other DNA
gcagcacaggtcctaaatag

SEQ ID NO: 46 - DNA, 21 bp, synthetic construct
  misc_feature 1..21: note = Synthetic Polynucleotide
  source 1..21: mol_type = other DNA
gggcaataaggtagtgaacag

SEQ ID NO: 47 - DNA, 18 bp, synthetic construct
  misc_feature 1..18: note = Synthetic Polynucleotide
  source 1..18: mol_type = other DNA
ctgtcccaacccccaaag

SEQ ID NO: 48 - DNA, 20 bp, synthetic construct
  misc_feature 1..20: note = Synthetic Polynucleotide
  source 1..20: mol_type = other DNA
acgtattcttccgggcagaa

SEQ ID NO: 49 - DNA, 23 bp, synthetic construct
  misc_feature 1..23: note = Synthetic Polynucleotide
  source 1..23: mol_type = other DNA
ctggtgaaaaggacctctcgaag

SEQ ID NO: 50 - DNA, 24 bp, synthetic construct
  misc_feature 1..24: note = Synthetic Polynucleotide
  source 1..24: mol_type = other DNA
ccagtttcactaatgacacaaacg

SEQ ID NO: 51 - DNA, 20 bp, synthetic construct
  misc_feature 1..20: note = Synthetic Polynucleotide
  source 1..20: mol_type = other DNA
agcaaagaccccaacgagaa

SEQ ID NO: 52 - DNA, 15 bp, synthetic construct
  misc_feature 1..15: note = Synthetic Polynucleotide
  source 1..15: mol_type = other DNA
ggcggcggtcacgaa

SEQ ID NO: 53 - DNA, 22 bp, synthetic construct
  misc_feature 1..22: note = Synthetic Polypeptide
  misc_feature 1: note = modified by 6FAM
  misc_feature 22: note = modified by TAMRA
  source 1..22: mol_type = other DNA
cgcgatcacatggtcctgctgg

SEQ ID NO: 54 - DNA, 327 bp, synthetic construct; identical to SEQ ID NO: 1
  misc_feature 1..327: note = Synthetic Polynucleotide
  source 1..327: mol_type = other DNA

SEQ ID NO: 55 - DNA, 269 bp, synthetic construct
  misc_feature 1..269: note = Synthetic Polynucleotide
  source 1..269: mol_type = other DNA
agggctctgcgtttgctccaggtagtccgctgctcccttgggcctgggcccactgacagccctggtgcctctggccggctgcacacctcctggcgggcagctgtgaatggtccacatttcagggactgttctggcaatacctggtccctgatttatggaccattcacggaggcctgccctgactgcccacggtgccgtggccaaagaggatctaagggcaccgctgagggcctacctaaccatcgtggggaataaggacagtgtcaccc

SEQ ID NO: 56 - DNA, 265 bp, synthetic construct
  misc_feature 1..265: note = Synthetic Polynucleotide
  source 1..265: mol_type = other DNA
agggctctgcgtttgctccaggtagtccgctgctcccttgggcctgggcccactgacagccctggtgcctctggccggctgcacacctcctggcgggcagctgtgacaaattagctgtcagcgctgttctggcaatacctggcgctgactgcaaatttgtcacggaggcctgccctgactgcccacggtgccgtggccaaagaggatctaagggcaccgctgagggcctacctaaccatcgtggggaataaggacagtgtcaccc

SEQ ID NO: 57 - protein, 8 aa, synthetic construct
  REGION 1..8: note = Synthetic Polypeptide
DDDDDDDD

SEQ ID NO: 58 - protein, 9 aa, synthetic construct
  REGION 1..9: note = Synthetic Polypeptide
DDDDDDDDD

SEQ ID NO: 59 - protein, 10 aa, synthetic construct
  REGION 1..10: note = Synthetic Polypeptide
DDDDDDDDDD

SEQ ID NO: 60 - protein, 11 aa, synthetic construct
  REGION 1..11: note = Synthetic Polypeptide
DDDDDDDDDDD

SEQ ID NO: 61 - protein, 12 aa, synthetic construct
  REGION 1..12: note = Synthetic Polypeptide
DDDDDDDDDDDD

SEQ ID NO: 62 - protein, 13 aa, synthetic construct
  REGION 1..13: note = Synthetic Polypeptide
DDDDDDDDDDDDD

SEQ ID NO: 63 - protein, 14 aa, synthetic construct
  REGION 1..14: note = Synthetic Polypeptide
DDDDDDDDDDDDDD

SEQ ID NO: 64 - protein, 5 aa, synthetic construct
  REGION 1..5: note = Synthetic Polypeptide
GGGGS

SEQ ID NO: 65 - protein, 25 aa, synthetic construct
  REGION 1..25: note = Synthetic Polypeptide
  REGION 3..25: note = MISC_FEATURE - may be absent
GGGGGGGGGGGGGGGGGGGGGGGGG

SEQ ID NO: 66 - protein, 26 aa, synthetic construct
  REGION 1..26: note = Synthetic Polypeptide
  REGION 3..25: note = MISC_FEATURE - may be absent
GGGGGGGGGGGGGGGGGGGGGGGGGS

SEQ ID NO: 67 - protein, 50 aa, synthetic construct
  REGION 1..50: note = Synthetic Polypeptide
  REGION 5..50: note = MISC_FEATURE - may be absent
GSGSGSGSGSGSGSGSGSGSGSGSGSGSGSGSGSGSGSGSGSGSGSGSGS

SEQ ID NO: 68 - protein, 100 aa, synthetic construct
  REGION 1..100: note = Synthetic Polypeptide
  REGION 9..100: note = MISC_FEATURE - may be absent
GGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 69 - DNA, 12322 bp, Homo sapiens
  source 1..12322: mol_type = genomic DNA
ctcacaaccagccgactctcccattatccagctgcctagtttggtgcttcaatgtacatggctattccgtgtgcatatgtgtgtatacaaacacgcatgcatgcctggatggacatacgtatgcacaggttattttttaaggacaattctttcaataaggtctttaccccttacttgaaacaggtgttcatgaaaaaaatgcacaaaatcctgcctggccggaataattcatgaagaaggggctggatccgtgggtcagagaacacaggaccagtttgccatcccaaggccgaaggattcgaggcacaaacccagcagcctcaacctagttcatggaggagcctcgcggggtcctggccaagcaagcccgcccctctggtgggaagagcggcgcctaggtggagggtggctgccgtaggagtggacatgaatgctggctttcagagagaacagcgtttcagttttggtcatcggaagtggtgccttcagcacagaagaagagcgtgatttctcctccaaggccgttgatctccaacccagaactaaaggggagaagagccacccccagcatccagcgtggcatctcttgtgccaggaccagggatgactgggccatggacacagatgtctccaaccttcaaccgtttgcatagcacacgggggactcgtgggggccacctgccactgccagctgaaacaatacaatggcaatactgacatccttcatgacgttttcccgacagacattcaggcagaaagtgctggtgcgttttctgtctgcaaagtagagggccatgcctcaccaatagaatagcgtgggccctgatgacctgctccgagtccactcacagccagtgacacttgcaaaaaactcccaaagccgtcttgggtttggctcccacagctcttgaccaatgtggccaaagctggacacctccttgggacactgggattattcataaatgcagcccgccctgactctccctgaatagcatctgaagtctttgtgaaggtcatggatcctgaacaaagtgtcaagggcaccaagaaggctgagggaagtccccggaagcggctgaccaaaggagaggccattcagaccagtgtttcttccagcgtcccatacccaggcagcggcacagctgccacccaagagagccccgcccaagagctcttagccccgcagcccttcccgggcccctcatcagttcttagggaaggctctcaggagaaaacgggccagcagcagaagccccccaaaaggccccccatcgaagcatccgtccacatctcacagcttccgcagcaccctctgacaccagcattcatgtcgcctggcaaacctgagcatctcctggaggggtccacatggcaactggttgaccccatgagacctggaccctctggctccttcgtggcccctgggctccatcctcagagccagctccttccttcccacgcttccatcattccccccgaggaccttcctggagtccccaaagtcttcgtgcctcgtccttcccaggtctccttgaagcccacagaagaggcacacaagaaggagaggaagccccagaagccaggcaagtacatctgccagtactgcagccggccctgtgccaagcccagcgtgctccagaagcacattcgctcacacacaggtgagaggccctacccctgcggcccctgtggcttctccttcaagaccaagagtaatctctacaagcacaggaagtcccatgcccaccgcatcaaagcaggcctggcctcaggcatgggtggcgagatgtacccacatgggctggagatggagcggatccctggggaagagtttgaggagcccactgagggagaaagcacagattctgaagaggagactagtgccacctctggtcaccctgcagagctctccccaagacccaagcagccccttctctccagcgggctatacagctctgggagccacagttccagccacgaacgctgttccctgtcccagtccagcacagcccagtcactcgaagacccccctccatttgtggaaccctcatctgagcaccccctgagccataaacctgaagacacccacacgattaagcagaagctggccctccgcttaagcgagaggaagaaggtgatcgatgagcaggcgtttctgagcccaggcagcaaagggagtactgagtctgggtatttctctcgctccgagagtgcagagcagcaggtcagccccccaaacaccaacgccaagtcctacgctgagatcatctttggcaagtgtgggcgaataggacagcggaccgccatgctgacagccacctccacccagcccctcctgcccctgtccaccgaagacaagcccagcctggtgcctttgtctgtaccccggacgcaggtgatcgagcacatcacgaagctcatcaccatcaacgaggccgtggtggacaccagcgagatcgacagcgtgaagccaaggcggagctcactgtccaggcgcagcagcatggagtccccaaaatccagcctctaccgggagcccctgtcatcccacagtgagaaaaccaagcctgaacaatcactgctgagcctccagcacccgcccagtaccgccccccctgtgcctctcctgagaagccactcaatgccttctgccgcctgcactatcagcaccccccaccaccccttccgaggtagctactccttcgatgaccatatcaccgactccgaagccctgagccacagcagtcacgtgtttacctcccacccccggatgctgaagcgccagccggcaatcgaattacctttgggaggggaatacagttctgaggagcctggcccaagcagcaaagacacagcctccaagccctcggacgaagtggaacccaaggaaagcgagcttaccaaaaagaccaagaagggtttgaaaacaaaaggggtgatctacgaatgtaacatatgtggtgctcggtacaagaaaagggataactacgaagcccacaaaaaatactactgctcagagcttcagatcgcaaagcccatctctgcaggcacccacacatctccagaagctgaaaagagtcagattgagcatgagccgtggtcccaaatgatgcattacaaactgggaaccaccctggaactcactccactgaggaagaggaggaaagagaagagccttggggacgaggaagagccacctgcctttgagtccacaaaaagtcagtttggcagccccgggccatctgatgctgctcggaaccttcccctggagtccaccaagtcaccagcagaaccaagtaaatcagtgccctccttggagggacccacgggcttccagccaaggactcccaagccagggtccggttcagaatcagggaaggagaggagaacaacgtccaaagaaatttctgtcatccagcacaccagctcctttgagaaatctgattctctcgagcagccgagtggcttggaaggggaagacaaacctctggcccagttcccatcacccccacctgccccacacggacgctctgctcactccctgcagcctaagttggtccgccagcccaacattcaggttcctgagatcctagtaactgaggagcctgaccggccggacacagagccagagccgccccctaaggaacctgagaagactgaggagttccaatggccccagcgcagccagacacttgcccagctcccagctgagaagctgccacccaaaaagaagaggttgcgcctggcagagatggcccaatcatcaggggagtccagcttcgagtcctctgtgcctctgtctcgcagcccgagccaggaaagcaatgtctctttgagtgggtccagccgctcagcctcgtttgagagggatgaccatgggaaagccgaggcccccagtccctcatctgacatgcgccccaaacccctgggcacccacatgttgactgtccccagccaccacccacatgcccgagagatgcggaggtcagcctcagagcagagccccaacgtttcccattctgcccacatgaccgagacacgcagcaaatcctttgactatggcagcttgtccttgacaggcccttctgctccagccccagtggctccaccagcgcgggtggccccgccagagagaagaaaatgcttcttggtgagacaggcctctctgagcaggcctccagaatctgagttggaggttgcccccaagggaagacaggagagcgaagaaccacagccctcatccagtaaaccctctgccaaaagctcattgtcccagatttcctctgcggccacctcacatggtggacccccgggaggcaagggcccagggcaggacaggcccccattggggcccactgtgccctacacagaagcactgcaagtgttccaccaccccgttgcccagacacccctgcatgagaagccatacctgcccccaccagtctcccttttctccttccagcatctcgtgcagcatgagccaggacagtctccagaattcttctccacccaggccatgtccagcctcctgtcctcaccatactccatgcccccacttcctccctccttatttcaagccccaccgcttcctctccagcctactgttctgcacccaggccaactccatctcccccagctcatgcctcacccagccaacatccccttcaggcagcccccttccttcctccccatgccatacccgacctcctcagcactgtcttctgggtttttcctgcctctgcaatcccagtttgcacttcagctccctggtgatgtggaaagccatctgccccagatcaaaaccagcctggccccactggcaacaggaagtgctggcctctcccccagcacagagtacagcagtgacatccggctaccccctgtggctcccccagccagctcctcagcacctacatcagctcctccactggccctgcctgcctgtccagacaccatggtgtccctggttgtgcctgtccgtgttcagaccaatatgccgtcctatgggagcgcaatgtacaccaccctttcccagatcttggtcacccagtcccaaggcagctcagcaactgtggcacttcccaagtttgaggaacccccatcaaaggggacgactgtatgtggtgcagatgtgcatgaggttgggcccggcccttctgggttaagtgaagagcaaagcagagctttcccaactccatacctgagagtgcctgtgacattacctgaaagaaaaggcacttccctgtcatcagagagtatcttgagcctggaggggagttcatcaacagcagggggaagcaaacgtgtcctttcaccagctggcagccttgaacttaccatggaaacccagcagcaaaaaagagtgaaggaggaggaggcttccaaggcagatgaaaaacttgagctggtaaaaccatgcagtgtggtcctaccagcaccgaggatgggaagaggccagagaaatcccacttaggcaaccagggccaaggcaggagggagctagaaatgctgtccagcctgtcctcagatccatctgacacaaaggaaattcctcccctccctcaccctgcattgtcccatgggacagccccaggctcagaagctttgaaggaatatccccagccatctggcaaacctcaccgaagagggttgaccccactgagcgtgaagaaagaagattccaaggaacaacctgatctcccctccttggcacctccgagctctctgcctctgtcagaaacgtcctccagaccagccaagtcacaagaaggtacggactcaaagaaggtactgcagttccccagcctccacacaaccactaatgtcagttggtgctatttaaactacattaagccaaatcacatccagcatgcagataggaggtcctctgtttacgctggttggtgcataagtttgtacaaccccaaccttccgggggtttccactaaagctgctttgtccctcctgaggtctaagcagaaagtgagcaaagagacatacaccatggccacagctccgcatcctgaggcaggaaggcttgtgccatccagctcccgcaagccccgcatgacagaggttcacctcccttcactggtttccccggaaggccagaaagatctagctagagtggagaaggaagaagagaggagaggggagccggaggaggatgctcctgcctcccagagaggggagccggcgaggatcaaaatcttcgaaggagggtacaaatcaaacgaagagtatgtatatgtgcgaggccgcggccgagggaaatatgtttgtgaggagtgtggaattcgctgcaagaagcccagcatgctgaagaaacacatccgcacccacactgacgtccggccctatgtgtgcaagcactgtcactttgcttttaaaaccaaagggaatctgactaagcacatgaagtcgaaggcccacagcaaaaagtgccaagagacaggggtgctggaggagctggaagccgaagaaggaaccagtgacgacctgttccaggactcggaaggacgagagggttcagaggctgtggaggagcaccagttttcggacctggaggactcggactcagactcagacctggacgaagacgaggatgaggatgaggaggagagccaggatgagctgtccagaccatcctcagaggcgcccccgcctggcccaccacatgcactgcgggcagactcctcacccatcctgggccctcagcccccagatgcccccgcctctggcacggaggctacacgaggcagctcggtctcggaagctgagcgcctgacagccagcagctgctccatgtccagccagagcatgccgggcctcccctggctgggaccggcccctctgggctctgtggagaaagacacaggctcagccttgagctacaagcctgtgtccccaagaagaccgtggtccccaagcaaagaagcaggcagccgtccaccactagcccgcaaacactcgctaaccaaaaacgactcatctccccagcgatgctccccggcccgagaaccacaggcctcagccccaagcccacctggcctgcacgtggacccaggaaggggcatgggcgctctcccttgtgggtctccaagacttcagctgtctcctctcaccctctgccccctgggaagagaactggcccctcgagcacatgtgctctccaaactcgagggtaccaccgacccaggcctccccagatactcgcccaccaggagatggtctccaggtcaggccgagtcaccaccacggtcagcgccgccagggaagtgggccttggctgggccgggcagcccctcagcgggggagcatggcccaggcttggggctggacccacgggttctcttcccgcccgcgcctctacctcacaagctcctcagcagaagcccagagacctgcgcctccccgtggaaggccgagtcccgaagtccctcctgctcacccggccctgctcatcctctctcctcccgacccttctccgccctccatgacttccacggccacatcctggcccggacagaggagaacatcttcagccacctgcctctgcactcccagcacttgacccgtgccccatgtcccttgattcccatcggtgggatccagatggtgcaggcccggccaggagcccaccccaccctgctgccagggcccaccgcagcctgggtcagtggcttctccgggggtggcagcgacctgacaggggcccgggaggcccaggagcgaggccgctggagtcccactgagagctcgtcagcctccgtgtcgcctgtggctaaggtctccaaattcacactctcctcagagctggagggcggggactaccccaaggagagggagaggaccggcggaggcccgggcaggcctcctgactggacaccccatgggaccggggcacctgcagagcccacacccacgcacagcccctgcaccccacccgacaccttgccccggccgccccagggacgccgggcagcgcagtcctggagcccccgcttgggtccccgcgtgcaccgaccaaccccgagccttctgccaccccgccgctggaccgcagcagctctgtgggctgcctggcagaggcctctgcccgcttcccagcccggacgaggaacctctccggggaacccaggaccaggcaggactcccccaagccctcaggaagtggggagcccagggcacatccacatcagcctgaggacagggttccccccaacgcttagcctctctccaactgcttcagcatctggcttccagtgtccagcaacagacgtttccagccactttcctcgaatcatcccacttcctcagccccatctgtccctccgtccaggagctctcacggccccatctgttgtaccttcccatgtatgcagttacctgtgcctttttctacaccttttgttgcttaaaaagaaacaaaacaaatcacatacatacatttaaaaaaaaaacaacaacccacgaggagtctgaggctgtgaatagtttatggttttggggaaaggctgatggtgaagcctcctgaccctccccgctgtggttggcagccacccaccccagaggctggcagagggaaaggggtacactgagggagaaaggaaaaggaaacttcaaacaatatagaattaaatgtaaaaggaagcactcctgtgtacagatgcgatcaaggttcctgtttattgccacttcacccccctgcccagctcgtagccacccctctctgccagcagaaaggccagtgtccccaggcagaggggcacaaacacaggcaggtgacccccacccaggccccagcaggcaggcccagaaaaactaatcttttccttttttttttttttttttttttttgcaagaaaataaaatgatacttttcctaggatttcaacacaaaataataggtgcaggtagaaggaggagggctggctccccaagggctcctggatactctggtagtctgagtcatgggcccatcctggcactccacaggtgggcaggccaccccacccacgcacccccactccagacacctcccttctgcaccccaccctggccccctgggctggggaaggagccctgactgtccgtccctggctcccaagcccctgaccgaggcctcactctcctgttgcctcctctgttctaaaaccaccaaaccacccacaaaggcagaagtggcagggcccgagccctagcggccgttcctgagactgggttttgggttttgtttcatcttggtccctggggtacaagggagcctgttcccctcatggctgggtttttccagttctccacagcagaggtttgcggggaactgtttcaggaccactttgccacaggaccgtttccccccgtccctgcccctgtctccactaccccaaggaaatacccacaactgtggctggtggatacggcctggacctgtttgctgtcttacacctcttttttaaaaagagagaggatggtgtttgatacttcacccagccaccacagattcttttgacctagaggatttttgaattgtcctaactcgttggaattctccaaagcaatcagtgtgagccagtgcctcttccttacccacatctctactttcaagaagctgccctgcatttcctggggcaaaactctactttgtaagaaaaataataggaccagaaatttaaatcccaaattgaactatggaacttgaactctagcgtgttcgccccaactgggagaggtgagctttttcccagtgtttcagaactgattttctttactttctacaagggagggcagcacagggactacggttgaggcccgtgaaggctgggtttgatgccaccctatacagagcagggacctctctggctaatccccagtcctcagccaggctgtgtgaatcaagtgcctgccccagggctcttgagctattgaagctgcttgggtacaggacacagtaggtggggagggttaagacccttctgtgagttccctgtgcggggctgtacttgcctcttccaattcgtggcctttccctgcttggtccctactagacagacaaaccagccacagtccagcctgcagccagaccaccttgttcactcattctcctttgcctcagagctaagacaaaaatgagacagaaggcagggctccctgggagtccactgtgctccagggttctggggaatcagggttagccagcagctcctggctgcttccctcagagactagggctctcatcctccccaagagaagcagcaagcccagcctggaccacactgtccatattgctggacagtggcctgacagaaagtgactcctccaagtcccaggaggccagggcttttctcatccttgcctttcagccctaacccatgggactgcccacggattggagacttcaagggctgaggtctgggagctgcataaagggcattgcttcagcccaggttagaaatctgcctgggcaagctcttcctgccccagacctacaaagcagcagaccgggggctctggtggactagcccctgacattggtggggggccccacaccactccaccccaccctgccttccagctctcctgggcatttttctccctgtactcaaacagcctacccacccaaggtttcctccctgggcagcctagcaatgaacagtgcagccggcagggcagaggcccggcagtcaccgggcccgtcaggctcaggcagagaagccacaggggccaggagtcactggagactatttctaaatgatgggggtaaatgcacaaatagaatctcaccaaagggctgcctccacattgatgccgtgcccagagggacagaaccaatgccaccagcctgggtatatgtcactgggcacagctctaaccccctcctccggactctagtcccgctcctctgcgcacagagcccccagcccacaggtacaccttcatgatttggagaaagacgctcgccccatgcacgccctcctctgggccttctgccctgctcccagtcacttccaagcttcctgtttgcctgtgatgttattgtgcctgttgagggaagcagcagaggaggcagtggctgacttggcacagatgcctgctacgtgctctgttgaaatgcgcggggtggccattcctcggtacagactagtcctggtccttgggtgtgggcagtgggggaggaaccaactggtcgaggtttcagagccaaaccttgcctttggttggtgagtccttgccccccaggcctgcgctccacgatgcctttcacccttggcaatctcagggccatcctgggtagtaaccccactcctctctgctcccgcccgcacctgtggctctcactctgggctcaacccctgcaaccctccaggagcccgacagcagccagctgcctgcactgtcgcctccgtaagctccaacttccagacccagaagtccctctgcttccctctgttggaaaaagcctaaaagaattagcttccagattcctctagcccctgctccattcccacccagtccttctgaagaggaatgagcaatacatctgagctggatttctctctagtcctttctccagacaaatccttcttaaagcaaaagtcctggctgagcacctgtccttggggaccgatctgccgtgtgaccaggggaagaaagttcccgaaagcctgttccaccaattctgcttctgtgttgtgaatccagtctgctttccattagaaaaccgcttcggcacttatggtcactttaataaatctagtatgtaaaaaaagaaagaaagaaaagaaacagaaaaaagaaacgtgcaggcaaatgtaaaatacaatgctctctgtaagataaatatttgcctttttttctaaaaggtgtacgtattctgtatgtgaaattgtctgtagaaagtttctatgttcttaaatggcaatacattccaaaaattgtactgtagatatgtacagcaaccgcactgggatggggtagttttgcctgtaattttatttaaactccagtttccacacttgctcttgcaatgttggtatggtatatatcagtgcaaaagaaaaaacaaaacagaaacaaacaaaaaaaaaaaacaaaaatccacgcaggtctaaagcacagagtctgacgtacaaaaggaaaaatgctcagtattgatgtgtgtgacctttgttgtaaattacatctgtactgtgaatgagaagtttttacaagtataataattgcctttattacagctctggctgagtgttcagcctgaggatattttttaaaaaaaaaagaattagcatgttggaataaatttgaaaatcccaacataaaaaaa

SEQ ID NO: 70 - protein, 2405 aa, Homo sapiens
MDPEQSVKGTKKAEGSPRKRLTKGEAIQTSVSSSVPYPGSGTAATQESPAQELLAPQPFPGPSSVLREGSQEKTGQQQKPPKRPPIEASVHISQLPQHPLTPAFMSPGKPEHLLEGSTWQLVDPMRPGPSGSFVAPGLHPQSQLLPSHASIIPPEDLPGVPKVFVPRPSQVSLKPTEEAHKKERKPQKPGKYICQYCSRPCAKPSVLQKHIRSHTGERPYPCGPCGFSFKTKSNLYKHRKSHAHRIKAGLASGMGGEMYPHGLEMERIPGEEFEEPTEGESTDSEEETSATSGHPAELSPRPKQPLLSSGLYSSGSHSSSHERCSLSQSSTAQSLEDPPPFVEPSSEHPLSHKPEDTHTIKQKLALRLSERKKVIDEQAFLSPGSKGSTESGYFSRSESAEQQVSPPNTNAKSYAEIIFGKCGRIGQRTAMLTATSTQPLLPLSTEDKPSLVPLSVPRTQVIEHITKLITINEAVVDTSEIDSVKPRRSSLSRRSSMESPKSSLYREPLSSHSEKTKPEQSLLSLQHPPSTAPPVPLLRSHSMPSAACTISTPHHPFRGSYSFDDHITDSEALSHSSHVFTSHPRMLKRQPAIELPLGGEYSSEEPGPSSKDTASKPSDEVEPKESELTKKTKKGLKTKGVIYECNICGARYKKRDNYEAHKKYYCSELQIAKPISAGTHTSPEAEKSQIEHEPWSQMMHYKLGTTLELTPLRKRRKEKSLGDEEEPPAFESTKSQFGSPGPSDAARNLPLESTKSPAEPSKSVPSLEGPTGFQPRTPKPGSGSESGKERRTTSKEISVIQHTSSFEKSDSLEQPSGLEGEDKPLAQFPSPPPAPHGRSAHSLQPKLVRQPNIQVPEILVTEEPDRPDTEPEPPPKEPEKTEEFQWPQRSQTLAQLPAEKLPPKKKRLRLAEMAQSSGESSFESSVPLSRSPSQESNVSLSGSSRSASFERDDHGKAEAPSPSSDMRPKPLGTHMLTVPSHHPHAREMRRSASEQSPNVSHSAHMTETRSKSFDYGSLSLTGPSAPAPVAPPARVAPPERRKCFLVRQASLSRPPESELEVAPKGRQESEEPQPSSSKPSAKSSLSQISSAATSHGGPPGGKGPGQDRPPLGPTVPYTEALQVFHHPVAQTPLHEKPYLPPPVSLFSFQHLVQHEPGQSPEFFSTQAMSSLLSSPYSMPPLPPSLFQAPPLPLQPTVLHPGQLHLPQLMPHPANIPFRQPPSFLPMPYPTSSALSSGFFLPLQSQFALQLPGDVESHLPQIKTSLAPLATGSAGLSPSTEYSSDIRLPPVAPPASSSAPTSAPPLALPACPDTMVSLVVPVRVQTNMPSYGSAMYTTLSQILVTQSQGSSATVALPKFEEPPSKGTTVCGADVHEVGPGPSGLSEEQSRAFPTPYLRVPVTLPERKGTSLSSESILSLEGSSSTAGGSKRVLSPAGSLELTMETQQQKRVKEEEASKADEKLELVKPCSVVLTSTEDGKRPEKSHLGNQGQGRRELEMLSSLSSDPSDTKEIPPLPHPALSHGTAPGSEALKEYPQPSGKPHRRGLTPLSVKKEDSKEQPDLPSLAPPSSLPLSETSSRPAKSQEGTDSKKVLQFPSLHTTTNVSWCYLNYIKPNHIQHADRRSSVYAGWCISLYNPNLPGVSTKAALSLLRSKQKVSKETYTMATAPHPEAGRLVPSSSRKPRMTEVHLPSLVSPEGQKDLARVEKEEERRGEPEEDAPASQRGEPARIKIFEGGYKSNEEYVYVRGRGRGKYVCEECGIRCKKPSMLKKHIRTHTDVRPYVCKHCHFAFKTKGNLTKHMKSKAHSKKCQETGVLEELEAEEGTSDDLFQDSEGREGSEAVEEHQFSDLEDSDSDSDLDEDEDEDEEESQDELSRPSSEAPPPGPPHALRADSSPILGPQPPDAPASGTEATRGSSVSEAERLTASSCSMSSQSMPGLPWLGPAPLGSVEKDTGSALSYKPVSPRRPWSPSKEAGSRPPLARKHSLTKNDSSPQRCSPAREPQASAPSPPGLHVDPGRGMGALPCGSPRLQLSPLTLCPLGRELAPRAHVLSKLEGTTDPGLPRYSPTRRWSPGQAESPPRSAPPGKWALAGPGSPSAGEHGPGLGLDPRVLFPPAPLPHKLLSRSPETCASPWKAESRSPSCSPGPAHPLSSRPFSALHDFHGHILARTEENIFSHLPLHSQHLTRAPCPLIPIGGIQMVQARPGAHPTLLPGPTAAWVSGFSGGGSDLTGAREAQERGRWSPTESSSASVSPVAKVSKFTLSSELEGGDYPKERERTGGGPGRPPDWTPHGTGAPAEPTPTHSPCTPPDTLPRPPQGRRAAQSWSPRLESPRAPTNPEPSATPPLDRSSSVGCLAEASARFPARTRNLSGEPRTRQDSPKPSGSGEPRAHPHQPEDRVPPNA